(19) United States
(12) Patent Application Publication
(10) Pub. No.: US 2021/0047627 A1
(43) Pub. Date: Feb. 18, 2021

(54) CRISPR-BASED DOWNREGULATION OF ALPHA-SYNUCLEIN EXPRESSION AS A NOVEL PARKINSON'S DISEASE THERAPEUTIC

(71) Applicant: PARKINSON'S INSTITUTE, Mountain View, CA (US)

(72) Inventors: Birgitt SCHÜLE, Mountain View, CA (US); Danuta SASTRE PHIPPS, Mountain View, CA (US); Carrolee BARLOW, Mountain View, CA (US); Tyler MOLITOR, Mountain View, CA (US)

(21) Appl. No.: 16/965,574

(22) PCT Filed: Jan. 29, 2019
(86) PCT No.: PCT/US2019/015715

§ 371 Date: Jul. 28, 2020

Related U.S. Application Data
(60) Provisional application No. 62/624,053, filed on Jan. 30, 2018.

(51) Int. Cl. C12N 9/22; C12N 15/113; G01N 33/50; A61K 31/7088; A61K 38/46; A61K 48/00

(52) U.S. Cl. CPC C12N 9/22; C12N 15/113; G01N 33/502; C12N 2800/80; A61K 38/465; A61K 48/0058; C12N 2310/20; A61K 31/7088

(57) ABSTRACT

Human-derived isogenic induced pluripotent stem cell (iPSCs) lines with copy number variation for alpha-synuclein, and methods of use thereof, are provided. Also disclosed are methods of modifying expression of alpha-synuclein gene using gene editing systems.

### CROSS REFERENCE

This application claims the benefit of U.S. Provisional Application No. 62/624,053 filed Jan. 30, 2018, which is incorporated herein by reference in its entirety.

### BACKGROUND OF THE INVENTION

Parkinson's disease (PD) represents one of the most common neurodegenerative diseases of aging. Approximately 1-2% of the population over 65 years of age is affected by this disorder, and it is estimated that the number of prevalent cases will double by the year 2030. The cause of the disease is unknown, but specific genetic susceptibility and/or exposure to environmental factors (e.g., pesticides or trauma) are likely to play a role. The accumulation and aggregation α-synuclein protein (α-syn) is a critical event in PD pathophysiology, impairing neuronal function and contributing to dopaminergic neuronal cell death. The pathogenic genomic triplication of the alpha-synuclein (SNCA) gene in patients results in early onset rapidly progressive Parkinsonism with diffuse Lewy body pathology and severe autonomic involvement, directly linking increased gene expression of wild-type α-syn and disease development.

### SUMMARY OF THE INVENTION

Disclosed herein, are methods of modifying expression of alpha-synuclein (SNCA) gene in an individual in need thereof, the method comprising: administering to the individual a composition comprising (i) at least one synthetic polynucleotide that targets a target sequence in one or more of the SNCA genes, and (ii) a nucleic acid-guided nuclease, wherein targeting the target sequence represses the transcription of one or more SNCA genes, thereby modifying expression of the SNCA gene in the individual. The individual may have a neurodegenerative disease. The individual may have Parkinson's disease, Parkinson's-related disease, or synucleinopathy. The individual may overexpress the SNCA gene. The individual may have more than two copies of a functional SNCA gene. The individual may have three copies of a functional SNCA gene. The individual may have four copies of a functional SNCA gene. The synthetic polynucleotide may be a guide nucleic acid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site of one or more SNCA genes. The nucleic acid-guided nuclease may be a CRISPR nuclease. The CRISPR nuclease may be Cas9. The CRISPR nuclease may be bacterial Cas9. The bacterial Cas9 may be from Staphylococcus aureus. The nucleic acid-guided nuclease may be catalytically inactive. The transcription may be repressed by interfering with transcription initiation, transcription elongation, RNA polymerase binding, transcription factor binding, or any combination thereof. Repressing the transcription of one or more SNCA genes may be reversible. Repressing the transcription of one or more SNCA genes may decrease the expression of the SNCA gene in the individual. The decreased expression of the SNCA gene may be comparable to the expression of SNCA gene in a control cell. The modified expression of SNCA gene may be comparable to the expression of SNCA gene in a control cell. The control cell may comprise two copies of functional SNCA gene. The transcription of SNCA gene may be repressed by at least 50% compared to transcription of SNCA gene before administration of the composition.

Disclosed herein, is method of treating a neurodegenerative disease in an individual in need thereof, the method comprising: administering to the individual a composition comprising (i) at least one synthetic polynucleotide that targets a target sequence in one or more of the SNCA genes, and (ii) a nucleic acid-guided nuclease, wherein the individual overexpresses SNCA gene, and wherein targeting the target sequence represses the transcription of one or more SNCA genes, thereby treating the individual. The neurodegenerative disease may be Parkinson's disease, Parkinson's-related disease, or synucleinopathy. The individual may have more than two copies of a functional SNCA gene. The individual may have three copies of a functional SNCA gene. The individual may have four copies of a functional SNCA gene. The synthetic polynucleotide may be a guide nucleic acid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site of one or more SNCA genes. The nucleic acid-guided nuclease may be a CRISPR nuclease. The CRISPR nuclease may be Cas9. The CRISPR nuclease may be bacterial Cas9. The bacterial Cas9 may be from Staphylococcus aureus. The nucleic acid-guided nuclease may be catalytically inactive. The transcription may be repressed by interfering with transcription initiation, transcription elongation, RNA polymerase binding, transcription factor binding, or any combination thereof. Repressing the transcription of one or more SNCA genes may be reversible. Repressing the transcription of one or more SNCA genes may decrease the expression of the SNCA gene in the individual. The decreased expression of the SNCA gene may be comparable to the expression of SNCA gene in a control cell. The control cell may comprise two copies of functional SNCA gene.

Disclosed herein, is method of measuring efficacy of a treatment for neurodegenerative disease in an individual overexpressing SNCA gene, the method comprising: (a) determining the copy number of SNCA gene in the individual; (b) contacting an isogenic induced pluripotent cell comprising a copy number of SNCA gene the same as the individual with a composition comprising (i) at least one synthetic polynucleotide that targets a target sequence in one or more SNCA genes, and (ii) a nucleic acid-guided nuclease; (c) detecting the response in the cell; and (d) comparing said response to control cells. The method may further comprise (e) adjusting the treatment to get a response comparable to the control cells. The method may further comprise (0 administering the composition with efficacy for treatment of the neurodegenerative to the individual. The neurodegenerative disease may be Parkinson's disease, Parkinson's-related disease, or synucleinopathy. The individual may have more than two copies of a functional SNCA gene. The individual may have three copies of a functional SNCA gene. The individual may have four copies of a functional SNCA gene. The synthetic polynucleotide may be a guide nucleic acid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site of one or more SNCA genes. The nucleic acid-guided nuclease may be a CRISPR nuclease. The CRISPR nuclease may be Cas9. The CRISPR nuclease may be bacterial Cas9. The bacterial Cas9 may be from Staphylococcus aureus. The nucleic acid-guided nuclease may be catalytically inactive. Targeting the target sequence may repress the transcription of one or more SNCA genes. The transcription may be repressed by interfering with transcription initiation, transcription elongation, RNA polymerase binding, transcription factor binding, or any combination thereof. Repressing the transcription of one or more SNCA genes may be reversible. The response may be change in cell viability, cellular chemistry, cellular function, mitochondrial function, cell aggregation, cell morphology, cellular protein aggregation, gene expression, cellular secretion, cellular uptake, or combinations thereof. The response may be detecting expression of one or more SNCA genes. The control cell may be an isogenic induced pluripotent cell comprising a copy number of SNCA gene the same as the individual without contact with the composition, or the control cell may be an isogenic induced pluripotent cell comprising two functional copies of SNCA gene without contact with the composition, or both.

Disclosed herein, is pharmaceutical composition for treatment of a neurodegenerative disease in an individual in need thereof, comprising (i) at least one synthetic polynucleotide that targets a target sequence in one or more of SNCA genes, and (ii) a nucleic acid-guided nuclease; and a pharmaceutically-acceptable excipient, wherein the composition has efficacy in the treatment of the neurodegenerative disease, wherein said efficacy is measured according to the methods disclosed herein. The neurodegenerative disease may be Parkinson's disease, Parkinson's-related disease, or synucleinopathy. The individual may overexpress the SNCA gene. The individual may have more than two copies of a functional SNCA gene. The individual may have three copies of a functional SNCA gene. The individual may have four copies of a functional SNCA gene. The synthetic polynucleotide may be a guide nucleic acid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site of one or more SNCA genes. The nucleic acid-guided nuclease may be a CRISPR nuclease. The CRISPR nuclease may be Cas9. The CRISPR nuclease may be bacterial Cas9. The bacterial Cas9 may be from Staphylococcus aureus. The nucleic acid-guided nuclease may be catalytically inactive.

Disclosed herein, is method of modifying expression of alpha-synuclein (SNCA) gene in an induced pluripotent stem cell, the method comprising: (a) providing induced pluripotent stem cell that overexpresses SNCA gene; and (b) contacting the stem cell with (i) at least one synthetic polynucleotide that targets a target sequence in one or more of the SNCA genes, and (ii) a nucleic acid-guided nuclease, wherein targeting the target sequence represses the transcription of one or more SNCA genes, thereby modifying expression of the SNCA gene. The cell may have more than two copies of a functional SNCA gene. The cell may have three copies of a functional SNCA gene. The cell may have four copies of a functional SNCA gene. The synthetic polynucleotide may be a guide nucleic acid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site of one or more SNCA genes. The nucleic acid-guided nuclease may be a CRISPR nuclease. The CRISPR nuclease may be Cas9. The CRISPR nuclease may be bacterial Cas9. The bacterial Cas9 may be from Staphylococcus aureus. The nucleic acid-guided nuclease may be catalytically inactive. The transcription may be repressed by interfering with transcription initiation, transcription elongation, RNA polymerase binding, transcription factor binding, or any combination thereof. Repressing the transcription of one or more SNCA genes may be reversible. Repressing the transcription of one or more SNCA genes may decrease the expression of the SNCA gene in the cell. The decreased expression of the SNCA gene may be comparable to the expression of SNCA gene in a control cell. The modified expression of SNCA gene may be comparable to the expression of SNCA gene in a control cell. The control cell may comprise two copies of functional SNCA gene. The cell may be present in a cell culture. The gRNA may modify the expression of the SNCA gene by suppressing the expression of the SNCA gene by 75%. The gRNA may modify the expression of the SNCA gene by suppressing the expression of the SNCA gene by 50%. The gRNA may be a gRNA according to the sequence CTCCTCTGGGGACAGTCCCCC (382R). The gRNA may be a gRNA according to the sequence AAGAGAGAGGCGGGGAGGAGT (267R). The gRNA may be a gRNA according to the sequence GAATGGTCGTGGGCACCGGGA (155R).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1C illustrate analysis of mitochondria respiratory chain complexes in neuroprecursor cells (NPCs). FIG. 1A illustrates a Western analysis of mitochondria respiratory chain complex IV, subunit I (C IV) expression in NPCs derived from Control (Ctrl) and SNCA-Triplication (SNCA-Tri) iPSCs. Heat shock protein 90 (HSP90) was used as loading control. Analysis of mitochondrial respiratory Complex I protein content (FIG. 1B) and Complex IV protein content and activity (FIG. 1C) in NPCs from Control (Ctrl), α-synuclein triplication (SNCA-Tri) under control conditions (HG), and after challenge with Rotenone (HG+Rot) or nutrient withdrawal (NG) determined by native ELISA based assays. Data from 2 independent experiments with 2 replicated each are shown (+/−SD, *p≤0.01, **p≤0.001; ANOVA).

FIG. 2 illustrates the GeneArt Genomic Cleavage Detection results for HEK293 cells transfected with pGS-U6-gRNA and pGS-CMV-hCas9 plasmids. Top shows gel electrophoresis of enzyme digested DNA fragments. Bottom shows the comparison of indel mutation % values from TIDE analysis and GeneArt Genomic Cleavage Kit.

FIG. 3A-FIG. 3B illustrate a methodology used to target SNCA locus. FIG. 3A illustrates the SNCA genomic locus of Chr.4q22.1 and CRISPR targeted gene region of the SNCA gene exon 2. FIG. 3B illustrates a novel concept for SNCA gene knockout iPSC model. Introduce sequentially guided by CRISPR frame-shift mutations via non-homologous end-joining in the first coding exon of the SNCA gene to generate an ‘SNCA gene dosage’ model at its endogenous locus.

FIG. 4 illustrates Cel-1 assay used to measure reagent (sgRNA) cutting efficiency in HEK293T. HEK293 were transfected with each reagent to assess cutting at the target locus via Cel-1 assay. All five reagents showed cutting efficiency at 10-13%. Lanes 1-5 represent reagents 1 through 5 and lane 6 represents the uncut control.

FIG. 5 illustrates cells were transfected with CRISPR3 (highest cutting reagent). In order to increase the chances of getting all four alleles cut, transfection with the nucleases was performed 3 times sequentially over 6 weeks of time period. Cel-1 assay showing increased cutting efficiency after three consecutive transfections on pooled human iPSCs with SNCA genomic triplication. Lanes 1, 2 and 3 represent cutting with CRISPR3 after each round, with cutting levels reaching 24.5% after the third round. Lanes 4 and 5 represent cutting with another pulse of gRNA 24 hours after the initial transfection. Cutting efficiency reaches 35% with the additional pulse of gRNA. Lane 7 represents the uncut control cells.

FIG. 6 illustrates SNCA expression in isogenic clones by determining SNCA mRNA expression in iPSCs. CRISPR knock-out (KO) clones are compared to normal control and parental SNCA triplication iPSC clones. Isogenic lines (1^{st} set) show mRNA reduction in proportion to number of KO copies. However, when compared to the parental SNCA triplication, the KO lines express proportionally higher levels of α-syn. There is still residual expression in the SNCA 4KO. Two other 2KO clones (2^{nd} set) show similar SNCA expression as control. To note, cell pellets for clones were collected to extract RNA at separate set.

FIG. 7 illustrates a schematic for timeline for neural induction of edited iPSCs and their differentiation into mature neurons. Media composition and added supplements (suppl.) are abbreviated as follows: SB43: SB431542; Dor: Dorsomorphin; Putr: Putrescin; Transf.: Transferrin; Na-Sel.: Na-Selenite; Ins.: Insulin.

FIG. 8 illustrates a schematic for differentiation of SNCA isogenic iPSC clones into dopaminergic (DA) neurons to investigate phenotypic differences among lines. FIG. 9 illustrates neuronal differentiation protocol exhibits homogenous population of floorplate marker forkhead box A2 (FOXA2). Upper panel exhibits pluripotent morphology of iPSCs. Lower panel exhibits morphology of cells after 10 days treated with specification media.

FIG. 10 illustrates FPp0 at 24 hours after passaging from day10. Cells uniformly express midbrain marker FOXA2 and neuro-precursor marker, NESTIN. Counterstained with DAPI at 10× magnification.

FIG. 11 illustrates expression of FOXA2 and tyrosine hydroxylase (TH) in DA neurons after 35 days of differentiation. DA neurons are still highly positive FOXA2. Co-localization of FOXA2 and TH confirms FOXA2 expression is critical to develop DA neurons. In 2F4_4KO line, even though high expression of FOXA2 is noticed, but TH expression is very limited. Cells were counterstained with DAPI at 10× magnification.

FIG. 12 illustrates mature day35 DA neurons stained with TUJ1 for neurons over total cell number. Cells were also double stained with TH for DA neurons. TH positive cells were co-localized with TUJ1 stained cells. Higher density of DA neurons is observed in control, compared to triplication and all isogenic lines. However, among all isogenic lines, DA neurons of 2KO line view morphological and quantity similarity with control. Cells were counterstained with DAPI at 10× magnification.

FIG. 13A-FIG. 13B illustrate SNCA and TH expression analysis for different time-points during neuronal differentiation. SNCA and TH mRNA expression at day0 (iPSCs), day10 (FPp0) and day35 (mature neurons) determined by Taqman qPCR. FIG. 13A illustrates SNCA expression is almost 60-70 fold higher at day35 compared to day0 or day10. FIG. 13B illustrates TH expression is 100 to 2000 fold higher compared to day0 and day10. At day 35, higher TH noted in 1KO, 2KO, and 3KO lines compared to SNCA triplication line.

FIG. 14A-FIG. 14D illustrate transcriptome analysis of the isogenic iPSC lines. FIG. 14A is a statistic chart of differentially expressed genes of each pair. Only >2-fold changes are presented. The first two columns of the graph compare the parental iPSC lines to the CRISPR knockout line with 2 frameshift mutations (2KO). 401 genes were downregulated, and 411 genes were upregulated. The middle two columns compare the parental iPSC lines to the CRISPR knockout line with 4 frameshift mutations (4KO). 156 were upregulated and 231 genes were downregulated. The last two columns compare the parental iPSC lines to the CRISPR knockout line with 3 frameshift mutations (3KO). 443 were upregulated and 807 genes were downregulated. FIG. 14B is gene ontology analysis of differentially expressed genes. CRISPR knockout line (1F6) with 2 frameshift mutations (2KO) is compared to the parental control. Several biological processes, cellular components, and molecular function have been identified to be affected due to a 2-fold change in SNCA expression. FIG. 14C is pathway enrichment analysis of differentially expressed genes. The left panel shows the top 20 pathways enriched in CRISPR knockout line with 2 frameshift mutations (2KO) compared to parental control. The signaling pathways are: signaling pathways regulating pluripotency genes, Ras signaling pathway, proteoglycans in cancer, protein digestion and absorption, PI3K/AKT signaling pathway, p53 signaling pathway, osteoclast differentiation, notch signaling pathway, mineral absorption, microRNAs in cancer, MAPK signaling pathway, Linoleic acid metabolism, FoxO signaling pathway, ether lipid metabolism, ECM-receptor interaction, choline metabolism in cancer, cell adhesion molecules, axon guidance, arachidonic acid and metabolism, alpha-linolenic acid metabolism. The left panel shows the top 20 pathways enriched in CRISPR knockout line with 4 frameshift mutations (2KO) compared to parental control. The signaling pathways are: Wnt signaling pathway, toxoplasmosis, Toll-like receptor signaling pathway, signaling pathways regulating pluripotency genes, rheumatoid arthritis, proteoglycans in cancer, pertussis, p53 signaling pathway, NF-kappa B signaling pathway, microRNAs in cancer, Leishmaniasis, Legionellosis, HTLV-1 infection, Hedgehog signaling pathway, FoxO signaling pathway, complement and coagulation cascades, Chagas disease, cell adhesion molecules, axon guidance. FIG. 14D is a prediction and annotation of novel transcripts. Each column presents an iPSC clone: the first column is CRISPR knockout line (1F6) with 2 frameshift mutations (2KO), the second column is CRISPR knockout line (2F4) with 4 frameshift mutations (4KO), the third column is CRISPR knockout line (4F6) with 3 frameshift mutations (3KO), the last column is the parental control (HUF4).

FIG. 15 illustrates an exemplary concept of development of proposed therapeutic approach. Using CRISPR/Cas9 mutant protein and sgRNA targeted to SNCA promoter.

FIG. 16 illustrates UC Santa Cruz genomic alignment of 50 sgRNAs with highest predicted scores. SNCA gene has three isoforms with individual transcription start sites. A total of 124 predicted sgRNAs with combined efficacy and specificity score between 15-20 were found.

FIG. 17A-FIG. 17D illustrate dopaminergic differentiation of human iPSCs. FIG. 17A illustrates expression of midbrain transcription factors. FIG. 17B illustrates dopaminergic markers during differentiation. FIG. 17C-FIG. 17D illustrate immunocytochemistry for tyrosine hydroxylase and beta-III-tubulin, 60% dopaminergic neurons.

FIG. 18A-FIG. 18D illustrate multielectrode arrays (MEA) of SNCA triplication dopaminergic neurons. FIG. 18A is an overview of morphology of neurons on MEA plate. FIG. 18B depicts an overall network activity. FIG. 18C illustrates spike activity pattern on electrodes. FIG. 18D illustrates measurements started 2 days after replating of 30 day-old dopaminergic neurons and shows overall number of spikes is higher compared to standard protocol.

FIG. 19 illustrates an exemplary epigenetic strategy to reduce alpha-synuclein expression by CRISPR interference. Inhibition of alpha-synuclein transcription may prevent neurodegeneration.

FIG. 20 is an exemplary schematic depicting gene repression without cuts in DNA and the reversible interaction with DNA using catalytically dead Cas9 (dCas9).

FIG. 21 illustrates the expression vectors used (dCas9, sgRNA and rtTA) in transfection of HEK293 cells.

FIG. 22 illustrates the triple transfection efficiency of dCas9, sgRNA and rtTA expression vectors.

FIG. 23 is an exemplary transient transfection workflow.

FIG. 24A-FIG. 24C illustrate graphs identifying sgRNA with at least 50% reduction in SNCA mRNA.

FIG. 25 illustrates graph with relative expression of SNCA mRNA in mixed versus pure populations of cells.

FIG. 26 is an exemplary workflow for generation of clonal SadCas9-2KRAB::tdTomato cell lines. This workflow was performed for iPSCs and HEK293T cells.

FIG. 27A-FIG. 27B are Flow cytometry scatter plots and sorting gates for establishment of SadCas9 iPSC lines H4C2 (FIG. 27A, SNCA triplication) and H5C2 (FIG. 27B, control sibling). Cells were infected with SadCas9 and rtTA, expanded for a week and then treated with DOX prior to sorting for tdTomato fluorescence (red dots in scatter plots). Percentage of SadCas9-tdTomato positive cells varied from 12.21% (H4C2) to 21.63% (H5C2).

FIG. 28 illustrates downregulation of SNCA mRNA in heterogeneous sadCas9 SNCA triplication iPSCs (H4C2) infected with control sgRNA (Gal4) and anti-SNCA sgRNAs. Downregulation of SNCA mRNA expression reached >1=75% (higher as compared to HEK293T cells). Relative expression of SNCA mRNA is represented on the y-axis, with sgRNAs used being represented in the x-axis.

FIG. 29 illustrates SNCA downregulation in clonal SadCas9-HEK293T cells with several sgRNAs against TSS1, TSS2, and TSS3. Relative expression of SNCA mRNA is represented on the y-axis, with sgRNAs used being represented in the x-axis.

FIG. 30 illustrates H4C2B clonal iPSCs show different levels of SadCas9 expression.

FIG. 31 illustrates H4C2B iPSCs maintain pluripotency (OCT4) after lentiviral transductions & single cell isolation.

FIG. 32 illustrates SNCA mRNA downregulation in clonal SNCA-trip human iPSC H4C2B. Downregulation only occurs in the presence of DOX. Relative expression of SNCA mRNA is represented on the y-axis, with sgRNAs used being represented in the x-axis, with both the untreated and doxycylin-treated groups.

FIG. 33 is an exemplary timeline for differentiation of dopaminergic neurons. Cells were treated on day 11 with 1 ug/mL DOX, and floor plate progenitor cells were collected at day 13 of differentiation for analysis of SNCA mRNA.

FIG. 34 illustrates mRNA expression of SNCA and FOXA2 in H4C2B-derived Floor plate progenitor cells after 13 days of differentiation into dopaminergic neurons.

FIG. 35A-FIG. 35C is an overview of selected sgRNAs for off-target analysis.

FIG. 36 illustrates alpha-synuclein gene isoform expression in SNCA triplication neuronal cultures (clone H4C2B) post-treatment with doxycycline (1 μg/mL) for 5 days prior to cell harvest. Relative expression was measured by Q-PCR and normalized to expression of hGAPDH. Calibrator sample was Gal4, a sgRNA designed against a prokaryotic gene not found in mammalian cells. Data are displayed as mean ±SEM for 2 independent biological samples and 3 technical replicates (n=6). Differences between groups were detected by ANOVA (p<0.0005***, p<0.0001****).

FIG. 37 is an exemplary workflow for functional assays.

FIG. 38 illustrates Caspase 3/7 endpoint assay. The figures illustrates caspase 3/7, Cas9, and far red dead cell stain for three sgRNA conditions. The upper panel represent the control Gal4, the middle panel shows reduced caspase induction with sgRNA 228R (50% alpha-synuclein reduction) and the lower panel shows negligible caspase 3/7 expression in sgRNA 382R (75% alpha-synuclein downregulation).

FIG. 39 illustrates CellROX® Oxidative Stress assay. The figure illustrates measurement of reactive oxygen species (ROS) using a fluorogenic probe that presents with a strong fluorogenic signal upon oxidation and localizes to nuclei. The upper panel represent the control Gal4, the middle and lower panels show reduced ROS (˜60% of Gal4 signal) with sgRNA 228R (50% alpha-synuclein reduction) and ˜45% of the Gal4 signal in sgRNA 382R (75% alpha-synuclein downregulation).

FIG. 40 illustrates Lipid peroxidation assay. The figure illustrates measurement of oxidized lipids and non-oxidized lipids using a lipophilic BODIPY probe. The upper panel represent the control Gal4, the middle and lower panels shows sgRNA 382R and 510F both supporting a 75% alpha-synuclein mRNA downregulation.

FIG. 41 illustrates neuronal differentiation of all sgRNA iPSC clones through floorplate progenitor stage.

FIG. 42 illustrates AAV9 and AAV9B cisterna magna GFP injection: coronal sections.

FIG. 43 illustrates AAV9 and AAV9B lateral ventrical GFP injection: coronal sections.

FIG. 44 illustrates AAV9 and AAV9B striatum GFP injection: coronal sections.

FIG. 45 illustrates AAV-PHP.eb vector and expression cassette design.

FIG. 46 illustrates: Panel A—Panoramic view: stereotactic injection of AAV-PHP. eb-CAG:tdTomato virus in the striatum (2.2× [symbol] 10 [symbol] \hat{ }10 vgs 7 days after injection), Panel B—HOECHST 33342 counterstain, Panel C—direct fluorescence tdTomato, Panel D—merge. Scale 50 micrometer for B-D.

FIG. 47 illustrates: Panel A—Panoramic view: stereotactic injection of AAV-PHP.eb-CAG:tdTomato virus in the substantia nigra (2.2× [symbol] 10 [symbol] \hat{ }10 vgs 7 days after injection), Panel B—HOECHST 33342 counterstain, Panel C—direct fluorescence tdTomato, Panel D—merge. Scale 50 micrometer for B-D.

FIG. 48 illustrates RNAScope for alpha-synuclein (green) and saCas9 probes (purple; also recognizing mutant sadCas9) in human SNCA triplication cell culture. Upper panel shows normal SNCA expression with no sadCas9 activated. Middle panel shows activation of sadCas9 and expression of unspecific sgRNA Gal4 which has no effect on alpha-synuclein expression. Lower panel shows activation of sadCas9 and SNCA-specific sgRNA 382R resulting in profound downregulation of SNCA expression (red square) as previously shown with Taqman Q-PCR.

FIG. 49 illustrates total human alpha-synuclein protein levels in mouse brain from SNCA A53T transgenic mice compared to Alpha-synuclein knockout mice (KO) with different dilution factors.

FIG. 50 illustrates total human alpha-synuclein protein levels in mouse regions from SNCA A53T transgenic mice with different dilution factors.

FIG. 51 illustrates SYBR Green primer design for off-targets detected by in-silico homology screen.

### DETAILED DESCRIPTION OF THE INVENTION

### Overview

Disclosed herein, gene editing technology is used to intervene at the genomic/DNA level before alpha-synuclein is transcribed into RNA or translated into protein and block the transcription of the alpha-synuclein gene. While genetic engineering usually results in permanent changes of a gene or genomic region of interest, the gene engineering approach disclosed herein, called CRISPR interference, is reversible and does not introduce changes in the genomic sequence. The CRISPR/dCas9 mutant protein is guided by a small guide RNA specifically targeted to the promoter region of the SNCA gene and sterically hinders the transcription initiation and elongation of the RNA. Conceptually by inhibition or reduction of gene transcription, there is less mRNA product that is translated into functional alpha-synuclein protein. Further disclosed herein is the combination of gene editing technology with gene therapy to modify the expression of alpha-synuclein protein as a novel therapeutic approach for Parkinson's disease.

### Parkinson's Disease and Neurodegeneration

PD is a progressive neurodegenerative disorder affecting 1-2% of the population over 65. To date, all available treatments are only symptomatic, but halting or slowing down disease progression is a critical unmet medical need. While the classical clinical features of the disease include tremor, rigidity, bradykinesia, and postural instability, the disease also comprises a whole spectrum of clinical symptoms that go far beyond motor deficits, including reduction in sense of smell, sleep problems, autonomic dysfunction, psychiatric abnormalities, and cognitive decline, which are all part of the clinical syndrome. Furthermore, the disease exhibits a remarkable degree of clinical variability in terms of severity, age at onset, and gender difference, with a male/female ratio of approximately 2:1. Although PD represents mostly a sporadic disease, a number of Mendelian forms of parkinsonism that also exhibit typical alpha-synuclein positive Lewy bodies and neurites have been identified.

Neuropathologically, the cardinal features include loss of dopaminergic neurons in the substantia nigra and intracellular inclusions known as Lewy bodies, the major neuronal pathology associated with PD and dementia with Lewy bodies (DLB). In addition to neuronal cell loss, Lewy body pathology has been detected throughout the brainstem axis, such as substantia nigra, locus coeruleus, nucleus basalis of Meynert, dorsal motor nucleus of vagus, raphe nuclei, hypothalamus, nucleus of Edinger-Westphal, olfactory bulb, as well as the cerebral cortex, limbic system, and autonomic ganglia. The cell loss in these areas ranges from 30-90% compared to control tissue.

A major component of Lewy bodies is alpha-synuclein. With the advent of alpha-synuclein based immunohistochemistry, it has been possible to better characterize not only Lewy bodies, but also the wide-spread alpha-synuclein positive Lewy neuritic pathology that exists in both the central and peripheral nervous system, providing an increasingly well-defined neuroanatomical basis for the many non-motor features of this disease.

### Alpha-Synuclein in Parkinson's Disease

Although Parkinson's disease (PD) represents mostly a sporadic disease, familial forms of parkinsonism and causative mutations in genes are well documented and have been intensively investigated over the last 20 years. Neuropathologically, the typical changes include loss of dopaminergic neurons in the substantia nigra and intracellular inclusions known as Lewy bodies which are the neuropathological hallmark of PD. A major component of Lewy bodies is the protein alpha-synuclein (α-syn). Alpha-synuclein is critical for normal function of subcellular membrane systems such as mitochondria and mitochondria-associated membranes, and may be involved in signal transduction, membrane remodeling, stabilization, and function of membrane-associated proteins. The accumulation and aggregation α-synuclein protein (a-syn) is a critical event in Parkinson's disease (PD) pathophysiology, impairing neuronal function and contributing to dopaminergic neuronal cell death. The pathogenic genomic triplication of the alpha-synuclein (SNCA) gene (chromosomal locus 4q21, size 1.7Mb) in patients results in early onset rapidly progressive parkinsonism with diffuse Lewy body pathology and severe autonomic involvement, suggesting a direct link between increased gene expression of wild-type α-syn and disease development. Increased expression of wild-type α-syn alone may lead to neurodegeneration, is demonstrated not only in patients with duplications and triplications of the SNCA genomic locus, but also certain common genetic promoter or other non-coding variants of the SNCA gene (e.g. Rep-1 allele) which may upregulate α-syn expression. Furthermore, toxicant exposures (e.g. paraquat or MPTP) have been shown to be associated with Parkinson's disease and can experimentally lead to an increase α-syn protein levels resulting in neuronal cell death.

### Gene Isoforms of Alpha-Synuclein

Alpha-synuclein has three smaller gene isoforms as a result of alternative mRNA splicing. Alpha-synuclein 140 is the main complete protein, alpha-synuclein 112 and alpha-synuclein 126 are shorter proteins lacking exon 5 (C-terminus) and exon 3 (N-terminus), respectively. A third isoform is lacking both exon 3 and exon 5, resulting in a protein product of 96 amino acids. It has been shown that each of the three alternative isoforms aggregates significantly less than the canonical isoform SNCA140.

### Point Mutations and Genomic Multiplications of the Alpha-Synuclein Gene

Recently, two novel point mutations have been described to be causative for PD, SNCA p.H50G, and SNCA, p.G51D, which raises the number of SNCA point mutations to five in total. The finding that both qualitative and quantitative alterations in the alpha-synuclein gene are associated with the development of a parkinsonian phenotype indicates that amino acid substitutions as well as overexpression of wild-type alpha-synuclein are capable of triggering a clinicopathological process that is very similar to typical PD.

### Alpha-Synuclein Genetic Risk Variants

Association studies that investigated specific polymorphisms within the SNCA gene that may alter its expression have found an association with PD. More specifically, the NACP-Repl polymorphism of the SNCA promoter, a mixed dinucleotide repeat, has shown an association with PD. NACP-Rep1 alleles differed in frequency for cases and controls (P<0.001) and the long allele, 263 bp, was associated with PD (odds ratio, 1.43).

Additional evidence for a role of Rep1 comes from studies showing that the DNA binding protein and transcriptional regulator PARP-1 specifically binds to SNCA-Rep1. The PARPs catalyze the transfer of ADP-ribose to various nuclear proteins and are involved in a several cellular processes such as DNA repair, regulation of chromatin structure, transcriptional regulation, trafficking, cell death activation and others. Using a transgenic mouse model, the regulatory translational activity by increasing alpha-synuclein expression was demonstrated. Functionally, SNCA expression levels in postmortem brains suggest that the Rep-1 allele and SNPs in the 3′ region of the SNCA gene have a significant effect on SNCA mRNA levels in the substantia nigra and the temporal cortex.

Other association studies comparing PD cases and controls studied the entire SNCA gene and found significant correlations with SNPs in intron 4 and the 3′ untranslated region of the SNCA gene and the risk for PD, suggesting that other regions within the SNCA gene may also be functionally important for the development of PD. Moreover, SNCA expression levels in postmortem brain suggest that a SNP in the 3′ region of the SNCA gene may have a significant effect on SNCA mRNA levels in the substantia nigra.

### Animal Models Based on Neurotoxins

A number of toxicant-induced models of PD elicit alpha-synuclein pathology. For example, treatment with daily injections of the mitochondrial toxin MPTP on 5 consecutive days results in a significant increase in alpha-synuclein mRNA and protein levels in midbrain extracts and in an increase of alpha-synuclein-immunoreactive neurons in the substantia nigra and neurodegeneration. Alpha-synuclein up-regulation is also a feature of the herbicide paraquat mouse model. In mice exposed to weekly injections of paraquat over a period of three consecutive weeks, levels of alpha-synuclein were enhanced in the substantia nigra and accompanied by aggregate formation. In addition to toxicant-induced models, there have been numerous animal models developed that either knockout or overexpress alpha-synuclein, wild-type or mutant protein, as transgenes or delivered by viral vectors.

Neuroprotection through Alpha-Synuclein Downregulation

As a proof of concept of modulation of expression of specific genes leading to neuroprotection, the use of small interference RNAs (siRNA) in the brain has recently been shown be effective against endogenous murine alpha-synuclein, serotonin transporter (SERT), and mutant human Huntingtin. Alpha-synuclein siRNA knockdown resulted in neuroprotection in non-human primates against MPTP, thus in this model downregulation of alpha-synuclein, mRNA and protein protected neurons from degeneration and cell death.

Gene modification and manipulation as disclosed herein may lead to modified expression of alpha-synuclein or non-functional alpha-synuclein protein and may have a similar neuroprotective effect against alpha-synuclein.

Parkinson's disease is inexorably progressive. While symptomatic forms of therapy are available, proven disease modifying agents have yet to be discovered. Therefore developing innovative approaches to slow down or stop disease progression is needed.

Disclosed herein, are methods of modifying expression of SNCA gene using gene editing technology, for e.g. CRISPR interference, as a novel therapeutic approach for PD. Some of the advantages of this method are: 1) reversible and does not introduce changes in the genomic sequence, and 2) the amount of alpha-synuclein transcript can be regulated and titrated to adjust the protein levels to normal or physiological levels.

Further, disclosed herein, are methods combining gene modification and gene therapy as a novel therapeutic approach for PD. Gene therapy for PD currently relies on two strategies. The first one introduces enzymes for neurotransmitter synthesis and the second introduces neurotrophic factors to improve function of the remaining neurons. Therefore, both gene therapy concepts are in principle symptomatic and not disease modifying. The therapeutic approach of CRISPR/dCas9 mutant delivery via gene therapy disclosed herein, in contrast, act at the cause of the disease and is disease modifying. The methods disclosed herein also enable a therapeutic intervention at an early stage in the disease, at a time when there is little functional impairment, or before motor signs and symptoms are present.

### Certain Terminology

The terms “individual,” “patient,” or “subject” are used interchangeably. As used herein, they mean any mammal (i.e. species of any orders, families, and genus within the taxonomic classification animalia: chordata: vertebrata: mammalia). In some cases, the mammal is a human. None of the terms require or are limited to situation characterized by the supervision (e.g. constant or intermittent) of a health care worker (e.g. a doctor, a registered nurse, a nurse practitioner, a physician's assistant, an orderly, or a hospice worker).

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing or minimally causing undesirable biological effects or significantly interacting in a deleterious manner with any of the components of the composition in which it is contained.

### Gene Editing Technologies

Recent developments of technologies to permanently alter the human genome and to introduce site-specific genome modifications in disease relevant genes lay the foundation for therapeutic applications. These technologies are now commonly known as “genome editing.”

Current gene editing technologies comprise zinc-finger nucleases (ZFN), TAL effector nucleases (TALEN), and clustered regularly interspaced short palindromic repeats (CRISPR)/CRISPR-associated (Cas) system. All three technologies create a double-strand break which may then be repaired by either non-homologous end joining (NHEJ) or—when donor DNA is present—homologous recombination (HR), an event that introduces the homologous sequence from a donor DNA fragment.

The CRISPR/Cas9 nuclease system can be targeted to specific genomic sites by complexing with a synthetic guide RNA that hybridizes a nucleotide DNA sequence (protospacer) immediately preceding an NGG motif (PAM, or protospacer-adjacent motif) recognized by Cas9. CRISPR-Cas9 nuclease generates with high-efficiency double-strand breaks at defined genomic locations that are usually repaired by non-homologous end-joining (NHEJ), which is error-prone and resulting in frameshift mutations that lead to knock-out alleles of genes and dysfunctional proteins.

CRISPR/Cas9 dead RNA-Guided Endonuclease for Specific Control of Gene Expression

The catalytically dead Cas9 from a type II CRISPR system, which lacks nuclease activity, may control gene expression when complexed with guide RNA. The dead Cas9 (dCas9) generates a DNA recognition complex that does not cleave the DNA, but that may specifically interfere with transcriptional elongation, RNA polymerase binding, and/or transcription factor binding. This system does not alter the genome but modifies gene expression by steric hindrance and is reversible. In some cases, the system also does not need the host machinery to function, whereas with genome editing, the cell needs proper function of certain protein and pathway functions e.g. NHEJ or HDR.

Methods disclosed herein may use a Staphylococcus aureus dCas9. In some cases, the S. aureus dCas9 comprises one or more heterologous functional domains, such as transcription repressor or activator. In some cases, the dCas9 is pSLQ2840 pPB: CAG-Puro-WPRE PGK-VPR-tagBFP-SadCas9, as described in Gao et al., Complex transcriptional modulation with orthogonal and inducible dCas9 regulators, Nat Methods. 13(12):1043-1049 (2016), herein incorporated by reference in its entirety. The dCas9 may comprise D10A and H840A substitutions.

The CRISPR/Cas9 nuclease system may also be joined or otherwise be connected to one or more transcriptional regulatory proteins or domains (e.g. activation domain or repressor domain). The transcriptional regulatory domains correspond to targeted loci. Disclosed herein are methods and materials for localizing transcriptional regulatory domains to targeted loci by fusing, connecting or joining such domains to either Cas9 or to the gRNA.

### Transcriptional Activation Domain

A transcriptional activation domain may interact with transcriptional control elements and/or transcriptional regulatory proteins (i.e., transcription factors, RNA polymerases, etc.) to increase and/or activate transcription of a gene. The transcriptional activation domain may be, without limit, a herpes simplex virus VP16 activation domain, VP64 (which is a tetrameric derivative of VP16), a NFkappa B p65 activation domain, p53 activation domains 1 and 2, a CREB (cAMP response element binding protein) activation domain, an E2A activation domain, and an NFAT (nuclear factor of activated T-cells) activation domain. The transcriptional activation domain may be Gal4, Gcn4, MLL, Rtg3, GIn3, Oaf1, Pip2, Pdr1, Pdr3, Pho4, and Leu3. The transcriptional activation domain may be wild type, or it may be a modified version of the original transcriptional activation domain. An engineered Cas9-gRNA system may enable RNA-guided genome regulation in human cells by tethering transcriptional activation domains to either a dead Cas9 or to guide RNAs. The Cas9-activators may be created by fusing a transcriptional activation domain, e.g., from VP64, to the N-terminus or C-terminus of the catalytically dead Cas9 protein. The transcriptional activation domains may be fused on the N or C terminus of the Cas9. A dCas9-VP64 fusion activated transcription of reporter constructs when combined with gRNAs targeting sequences near the promoter, may display RNA-guided transcriptional activation.

### Transcriptional Repressor Domain

A transcriptional repressor domain may interact with transcriptional control elements and/or transcriptional regulatory proteins (i.e., transcription factors, RNA polymerases, etc.) to decrease and/or terminate transcription of a gene. Non-limiting examples of suitable transcriptional repressor domains include, but are not limited to, inducible cAMP early repressor (ICER) domains, Kruppel-associated box A (KRAB-A) repressor domains, YY1 glycine rich repressor domains, Sp1-like repressors, E(spI) repressors, Ikappa B repressor, MeCP2, NuE domain, NcoR domain, SID domain, and a SID4X domain. The transcriptional repressor domain may be wild type, or it may be a modified version of the original transcriptional repressor domain. An engineered Cas9-gRNA system may enable RNA-guided genome regulation in human cells by tethering transcriptional repressor domains to either a dead Cas9 or to guide RNAs. The Cas9-activators may be created by fusing a transcriptional repressor domain, e.g., KRAB domain, to the N-terminus or C-terminus of the catalytically dead Cas9 protein. The transcriptional repressor domains may be fused on the N or C terminus of the Cas9. A dCas9-KRAB fusion activated transcription of reporter constructs when combined with gRNAs targeting sequences near the promoter, may display RNA-guided transcriptional repression.

In addition, other heterologous functional domains (e.g., enzymes that modify the methylation state of DNA (e.g., DNA methyltransferase (DNMT) or TET proteins), or enzymes that modify histone subunit (e.g., histone acetyltransferases (HAT), histone deacetylases (HDAC), or histone demethylases)) as are known in the art may also be used.

### Targetable Nucleic Acid Cleavage Systems

Methods disclosed herein comprise targeting cleavage of specific nucleic acid sequences using a site-specific, targetable, and/or engineered nuclease or nuclease system. Such nucleases may create double-stranded break (DSBs) at desired locations in a genome or nucleic acid molecule. In other examples, a nuclease may create a single strand break. In some cases, two nucleases are used, each of which generates a single strand break.

The one or more double or single strand break may be repaired by natural processes of homologous recombination (HR) and non-homologous end-joining (NHEJ) using the cell's endogenous machinery. Additionally or alternatively, endogenous or heterologous recombination machinery may be used to repair the induced break or breaks.

Engineered nucleases such as zinc finger nucleases (ZFNs), Transcription Activator-Like Effector Nucleases (TALENs), engineered homing endonucleases, and RNA or DNA guided endonucleases, such as CRISPR/Cas such as Cas9 or CPF1, and/or Argonaute systems, are particularly appropriate to carry out some of the methods of the present disclosure. Additionally or alternatively, RNA targeting systems may be used, such as CRISPR/Cas systems including c2c2 nucleases.

Methods disclosed herein may comprise cleaving a target nucleic acid using CRISPR systems, such as a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. Alternatively, Class 2 systems include a single effector molecule and include Type II, Type V, and Type VI.

CRISPR systems used in methods disclosed herein may comprise a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence. CRISPR systems may comprise a single or multiple guiding RNAs. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs.

A gRNA may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some examples, the spacer sequence may be less than 10 or more than 36 nucleotides in length. A gRNA may comprise a repeat sequence. In some cases, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the spacer sequence may be less than 10 or more than 50 nucleotides in length.

A gRNA may comprise one or more synthetic nucleotides, non-naturally occurring nucleotides, nucleotides with a modification, deoxyribonucleotide, or any combination thereof. Additionally or alternatively, a gRNA may comprise a hairpin, linker region, single stranded region, double stranded region, or any combination thereof. Additionally or alternatively, a gRNA may comprise a signaling or reporter molecule.

A CRISPR nuclease may be endogenously or recombinantly expressed within a cell. A CRISPR nuclease may be encoded on a chromosome, extrachromosomally, or on a plasmid, synthetic chromosome, or artificial chromosome. A CRISPR nuclease may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

gRNAs may be encoded by genetic or episomal DNA within a cell. In some examples, gRNAs may be provided or delivered to a cell expressing a CRISPR nuclease. gRNAs may be provided or delivered concomitantly with a CRISPR nuclease or sequentially. Guide RNAs may be chemically synthesized, in vitro transcribed or otherwise generated using standard RNA generation techniques known in the art.

A CRISPR system may be a Type II CRISPR system, for example a Cas9 system. The Type II nuclease may comprise a single effector protein, which, in some cases, comprises a RuvC and HNH nuclease domains. In some cases a functional Type II nuclease may comprise two or more polypeptides, each of which comprises a nuclease domain or fragment thereof. The target nucleic acid sequences may comprise a 3′ protospacer adjacent motif (PAM). In some examples, the PAM may be 5′ of the target nucleic acid. Guide RNAs (gRNA) may comprise a single chimeric gRNA, which contains both crRNA and tracrRNA sequences. Alternatively, the gRNA may comprise a set of two RNAs, for example a crRNA and a tracrRNA. The Type II nuclease may generate a double strand break, which in some cases creates two blunt ends. In some cases, the Type II CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type II nickase is used to generate two single strand breaks, the resulting nucleic acid free ends may either be blunt, have a 3′ overhang, or a 5′ overhang. In some examples, a Type II nuclease may be catalytically dead such that it binds to a target sequence, but does not cleave. For example, a Type II nuclease may have mutations in both the RuvC and HNH domains, thereby rendering the both nuclease domains non-functional. A Type II CRISPR system may be one of three sub-types, namely Type II-A, Type II-B, or Type II-C.

A CRISPR system may be a Type V CRISPR system, for example a Cpfl, C2c1, or C2c3 system. The Type V nuclease may comprise a single effector protein, which in some cases comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some cases, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which in some cases generates a 5′ overhang. In some cases, the Type V CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type V nickase is used to generate two single strand breaks, the resulting nucleic acid free ends may either be blunt, have a 3′ overhang, or a 5′ overhang. In some examples, a Type V nuclease may be catalytically dead such that it binds to a target sequence, but does not cleave. For example, a Type V nuclease could have mutations a RuvC domain, thereby rendering the nuclease domain non-functional.

A CRISPR system may be a Type VI CRISPR system, for example a C2c2 system. A Type VI nuclease may comprise a HEPN domain. In some examples, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′or the target or protospacer sequence. Guide RNAs (gRNA) may comprise a single gRNA or single crRNA. In some cases, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some examples, a Type VI nuclease may be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some methods disclosed herein, Argonaute (Ago) systems may be used to cleave target nucleic acid sequences. Ago protein may be derived from a prokaryote, eukaryote, or archaea. The target nucleic acid may be RNA or DNA. A DNA target may be single stranded or double stranded. In some examples, the target nucleic acid does not require a specific target flanking sequence, such as a sequence equivalent to a protospacer adjacent motif or protospacer flanking sequence. The Ago protein may create a double strand break or single strand break. In some examples, when a Ago protein forms a single strand break, two Ago proteins may be used in combination to generate a double strand break. In some examples, an Ago protein comprises one, two, or more nuclease domains. In some examples, an Ago protein comprises one, two, or more catalytic domains. One or more nuclease or catalytic domains may be mutated in the Ago protein, thereby generating a nickase protein capable of generating single strand breaks. In other examples, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In many examples, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the gDNA may be less than 10 nucleotides in length. In some examples, the gDNA may be more than 50 nucleotides in length.

Argonaute-mediated cleavage may generate blunt end, 5′ overhangs, or 3′ overhangs. In some examples, one or more nucleotides are removed from the target site during or following cleavage.

Argonaute protein may be endogenously or recombinantly expressed within a cell. Argonaute may be encoded on a chromosome, extrachromosomally, or on a plasmid, synthetic chromosome, or artificial chromosome. Additionally or alternatively, an Argonaute protein may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

Guide DNAs may be provided by genetic or episomal DNA within a cell. In some examples, gDNA are reverse transcribed from RNA or mRNA within a cell. In some examples, gDNAs may be provided or delivered to a cell expressing an Ago protein. Guide DNAs may be provided or delivered concomitantly with an Ago protein or sequentially. Guide DNAs may be chemically synthesized, assembled, or otherwise generated using standard DNA generation techniques known in the art. Guide DNAs may be cleaved, released, or otherwise derived from genomic DNA, episomal DNA molecules, isolated nucleic acid molecules, or any other source of nucleic acid molecules.

Nuclease fusion proteins may be recombinantly expressed within a cell. A nuclease fusion protein may be encoded on a chromosome, extrachromosomally, or on a plasmid, synthetic chromosome, or artificial chromosome. A nuclease and a chromatin-remodeling enzyme may be engineered separately, and then covalently linked, prior to delivery to a cell. A nuclease fusion protein may be provided or delivered to the cell as a polypeptide or mRNA encoding the polypeptide. In such examples, polypeptide or mRNA may be delivered through standard mechanisms known in the art, such as through the use of cell permeable peptides, nanoparticles, or viral particles.

### Guide Nucleic Acid

A guide nucleic acid may complex with a compatible nucleic acid-guided nuclease and may hybridize with a target sequence, thereby directing the nuclease to the target sequence. A subject nucleic acid-guided nuclease capable of complexing with a guide nucleic acid may be referred to as a nucleic acid-guided nuclease that is compatible with the guide nucleic acid. Likewise, a guide nucleic acid capable of complexing with a nucleic acid-guided nuclease may be referred to as a guide nucleic acid that is compatible with the nucleic acid-guided nucleases.

A guide nucleic acid may be DNA. A guide nucleic acid may be RNA. A guide nucleic acid may comprise both DNA and RNA. A guide nucleic acid may comprise modified of non-naturally occurring nucleotides. In cases where the guide nucleic acid comprises RNA, the RNA guide nucleic acid may be encoded by a DNA sequence on a polynucleotide molecule such as a plasmid, linear construct, or editing cassette as disclosed herein.

A guide nucleic acid may comprise a guide sequence. A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some aspects, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is 10-30 nucleotides long. The guide sequence may be 10-25 nucleotides in length. The guide sequence may be 10-20 nucleotides in length. The guide sequence may be 15-30 nucleotides in length. The guide sequence may be 20-30 nucleotides in length. The guide sequence may be 15-25 nucleotides in length. The guide sequence may be 15-20 nucleotides in length. The guide sequence may be 20-25 nucleotides in length. The guide sequence may be 22-25 nucleotides in length. The guide sequence may be 15 nucleotides in length. The guide sequence may be 16 nucleotides in length. The guide sequence may be 17 nucleotides in length. The guide sequence may be 18 nucleotides in length. The guide sequence may be 19 nucleotides in length. The guide sequence may be 20 nucleotides in length. The guide sequence may be 21 nucleotides in length. The guide sequence may be 22 nucleotides in length. The guide sequence may be 23 nucleotides in length. The guide sequence may be 24 nucleotides in length. The guide sequence may be 25 nucleotides in length.

A guide nucleic acid may comprise a scaffold sequence. In general, a “scaffold sequence” includes any sequence that has sufficient sequence to promote formation of a targetable nuclease complex, wherein the targetable nuclease complex comprises a nucleic acid-guided nuclease and a guide nucleic acid comprising a scaffold sequence and a guide sequence. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment may be determined by any suitable alignment algorithm, and may further account for secondary structures, such as self-complementarity within either the one or two sequence regions. In some aspects, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some aspects, at least one of the two sequence regions is about or more than about 5, 6, 7, 8, 9, 10, 11, 12, 13, 14, 15, 16, 17, 18, 19, 20, 21, 22, 23, 24, 25, 30, 40, 50, or more nucleotides in length. In some aspects, at least one of the two sequence regions is about 10-30 nucleotides in length. At least one of the two sequence regions may be 10-25 nucleotides in length. At least one of the two sequence regions may be 10-20 nucleotides in length. At least one of the two sequence regions may be 15-30 nucleotides in length. At least one of the two sequence regions may be 20-30 nucleotides in length. At least one of the two sequence regions may be 15-25 nucleotides in length. At least one of the two sequence regions may be 15-20 nucleotides in length. At least one of the two sequence regions may be 20-25 nucleotides in length. At least one of the two sequence regions may be 22-25 nucleotides in length. At least one of the two sequence regions may be 15 nucleotides in length. At least one of the two sequence regions may be 16 nucleotides in length. At least one of the two sequence regions may be 17 nucleotides in length. At least one of the two sequence regions may be 18 nucleotides in length. At least one of the two sequence regions may be 19 nucleotides in length. At least one of the two sequence regions may be 20 nucleotides in length. At least one of the two sequence regions may be 21 nucleotides in length. At least one of the two sequence regions may be 22 nucleotides in length. At least one of the two sequence regions may be 23 nucleotides in length. At least one of the two sequence regions may be 24 nucleotides in length. At least one of the two sequence regions may be 25 nucleotides in length.

A scaffold sequence of a subject guide nucleic acid may comprise a secondary structure. A secondary structure may comprise a pseudoknot region. In some example, the compatibility of a guide nucleic acid and nucleic acid-guided nuclease is at least partially determined by sequence within or adjacent to a pseudoknot region of the guide RNA. In some cases, binding kinetics of a guide nucleic acid to a nucleic acid-guided nuclease is determined in part by secondary structures within the scaffold sequence. In some cases, binding kinetics of a guide nucleic acid to a nucleic acid-guided nuclease is determined in part by nucleic acid sequence with the scaffold sequence.

In aspects of the disclosure the terms “guide nucleic acid” refers to a polynucleotide comprising 1) a guide sequence capable of hybridizing to a target sequence and 2) a scaffold sequence capable of interacting with or complexing with a nucleic acid-guided nuclease as described herein.

A guide nucleic acid may be compatible with a nucleic acid-guided nuclease when the two elements may form a functional targetable nuclease complex capable of cleaving a target sequence. Often, a compatible scaffold sequence for a compatible guide nucleic acid may be found by scanning sequences adjacent to native nucleic acid-guided nuclease loci. In other words, native nucleic acid-guided nucleases may be encoded on a genome within proximity to a corresponding compatible guide nucleic acid or scaffold sequence.

Nucleic acid-guided nucleases may be compatible with guide nucleic acids that are not found within the nucleases endogenous host. Such orthogonal guide nucleic acids may be determined by empirical testing. Orthogonal guide nucleic acids may come from different bacterial species or be synthetic or otherwise engineered to be non-naturally occurring.

Orthogonal guide nucleic acids that are compatible with a common nucleic acid-guided nuclease may comprise one or more common features. Common features may include sequence outside a pseudoknot region. Common features may include a pseudoknot region. Common features may include a primary sequence or secondary structure.

A guide nucleic acid may be engineered to target a desired target sequence by altering the guide sequence such that the guide sequence is complementary to the target sequence, thereby allowing hybridization between the guide sequence and the target sequence. A guide nucleic acid with an engineered guide sequence may be referred to as an engineered guide nucleic acid. Engineered guide nucleic acids are often non-naturally occurring and are not found in nature.

### Gene Therapy for In Vivo Gene Editing Using CRISPR/Cas9

The delivery vehicles utilized in the methods disclosed herein for Cas9/CRISPR delivery in human brain for PD may be an adeno-associated virus (AAV) or lentiviral vector. Both viruses are used in clinical trials in humans for brain delivery and have been proven in phase I/II clinical trials to be safe and well tolerated. Other viral delivery options for the gene editing technology are also considered if deemed to be more advantageous over the aforementioned approaches.

Adeno-associated viral (AAV) vectors are excellent vehicles to transfer genes into the nervous system due to their property to transduce also post-mitotic cells, their ability to be grown to very high titers (up to 10¹³ virion particles per ml), and their relatively large insert capacity (insert capacity of about 7.5 kb). Adenoviral vectors may express the transgene(s) for a long time in the CNS in vivo and in cell culture such as neurons and glia. In the methods disclosed herein, AAV vector may be used as delivery vehicle. The AAV vector used may include, but are not limited to AAV1, AAV2, AAV4, AAV5, AAV6, AAV7, AAV8, AAV9, AAVrh.8, or AAVrh.10. The AAV vector used maybe AAV1. The AAV vector used maybe AAV2. The AAV vector used maybe AAV4. The AAV vector used maybe AAV5. The AAV vector used maybe AAV6. The AAV vector used maybe AAV8. The AAV vector used maybe AAV9. The AAV vector used maybe AAVrh.10.

Lentiviral vectors have shown to have a low immunogenicity, can transduce neurons, and may carry large inserts which allows for the introduction of multiple sgRNAs against several locations of one gene or multiple genes concurrently.

The CRISPR gene editing or gene regulation approach may be delivered in the brain via stereotactic surgery into the substantia nigra pars compacta.

In the methods disclosed herein, a dual-vector system is designed packaging SpCas9 (AAV-SpCas9) and the sgRNA (AAV-SpGuide) into two individual vectors which were co-transduced with an efficiency of ˜75%. Constitutive expression of Cas9 did not affect survival and morphology of neurons. The relatively large size of SpCas9 (˜4 kb coding sequence) may hinder the efficient packaging of plasmids carrying the SpCas9 cDNA into adeno-associated virus (AAV). Conversely, the coding sequence for Staphylococcus aureus Cas9 (SaCas9) is ˜1 kilobase shorter than SpCas9, allowing it to be efficiently packaged into AAV. The methods disclosed herein, also use another dual-vector system packaging Sa dCas9 (AAV-Sa dCas9) and the sgRNA (AAV-SaGuide) into two individual vectors which are co-transduced.

Isogenic Induced Pluripotent Cell (iPSC) Line with Different Functional Copy Numbers of the SNCA Gene

Disclosed herein, are isogenic iPSC lines produced from induced pluripotent stem cell with alpha-synuclein (SNCA) gene triplication. The isogenic iPSC line may have three copies of functional SNCA gene. The isogenic iPSC line may have two copies of functional SNCA gene. The isogenic iPSC line may have one copy of functional SNCA gene. The isogenic iPSC line may have zero copy of functional SNCA gene.

iPSC Line with Three Copies of Functional SNCA Gene

Disclosed herein, is isogenic iPSC line comprising three copies of alpha-synuclein (SNCA) gene, wherein the cell line is produced from induced pluripotent stem cell with alpha-synuclein (SNCA) gene triplication. The induced pluripotent stem cell with alpha-synuclein (SNCA) gene triplication may be human-derived. The SNCA gene may be a functional SNCA gene. The SNCA gene may be a wild-type SNCA gene. The functional SNCA gene may be a SNCA gene that encodes a protein with wild-type functionality. The functional SNCA gene may be a SNCA gene that encodes a fully functional α-syn protein. The functional SNCA gene may be a wild-type SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may maintain differentiation potential. The morphology of the cell during initial specification may be comparable to a control cell comprising two copies of the wild-type SNCA gene. The SNCA mRNA expression in the cell may be increased compared to SNCA mRNA expression in a control cell wherein the control cell comprises two copies of wild-type SNCA gene. The SNCA mRNA expression in the cell may be comparable to SNCA mRNA expression in a control cell wherein the control cell comprises two copies of wild-type SNCA gene. The cell may be present in a cell culture. The iPSC line with three copies of functional SNCA gene may be used to derive a neuronal precursor cell line. The iPSC line with three copies of functional SNCA gene may be used to derive a neuronal cell line. The derived neuronal cell line may be used to derive a dopaminergic (DA) neuron. The isogenic iPSC line may be used as cellular tool for in vitro studies to understand the molecular mechanism of α-syn under expression and overexpression in the pathogenesis of PD.

iPSC Line with two copies of Functional SNCA Gene

Disclosed herein, is isogenic iPSC line comprising two copies of alpha-synuclein (SNCA) gene, wherein the cell line is produced from induced pluripotent stem cell with alpha-synuclein (SNCA) gene triplication. The induced pluripotent stem cell with alpha-synuclein (SNCA) gene triplication may be human-derived. The SNCA gene may be a functional SNCA gene. The SNCA gene may be a wild-type SNCA gene. The functional SNCA gene may be a SNCA gene that encodes a protein with wild-type functionality. The functional SNCA gene may be a SNCA gene that encodes a fully functional α-syn protein. The functional SNCA gene may be a wild-type SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may maintain differentiation potential. The morphology of the cell during initial specification may be comparable to a control cell comprising two copies of the wild-type SNCA gene. The SNCA mRNA expression in the cell may be higher compared to SNCA mRNA expression in a control cell wherein the control cell comprises two copies of wild-type SNCA gene. The SNCA mRNA expression in the cell may be comparable to SNCA mRNA expression in a control cell wherein the control cell comprises two copies of wild-type SNCA gene. The cell may be present in a cell culture. The iPSC line with two copies of functional SNCA gene may be used to derive a neuronal precursor cell line. The iPSC line with two copies of functional SNCA gene may be used to derive a neuronal cell line. The derived neuronal cell line may be used to derive a dopaminergic (DA) neuron. The isogenic iPSC line may be used as cellular tool for in vitro studies to understand the molecular mechanism of α-syn under expression and overexpression in the pathogenesis of PD.

iPSC Line with One Copy of Functional SNCA Gene

Disclosed herein, is isogenic iPSC line comprising one copy of alpha-synuclein (SNCA) gene, wherein the cell line is produced from induced pluripotent stem cell with alpha-synuclein (SNCA) gene triplication. The induced pluripotent stem cell with alpha-synuclein (SNCA) gene triplication may be human-derived. The SNCA gene may be a functional SNCA gene. The SNCA gene may be a wild-type SNCA gene. The functional SNCA gene may be a SNCA gene that encodes a protein with wild-type functionality. The SNCA gene may be a SNCA gene that encodes a fully functional α-syn protein. The functional SNCA gene may be a wild-type SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may maintain differentiation potential. The cell may have reduced differentiation potential compared to a control cell, wherein the control cell comprises two copies of wild-type SNCA gene. The morphology of the cell during initial specification may be comparable to a control cell comprising two copies of the wild-type SNCA gene. The SNCA mRNA expression in the cell may be higher compared to SNCA mRNA expression in a control cell wherein the control cell comprises two copies of wild-type SNCA gene. The SNCA mRNA expression in the cell may be comparable to SNCA mRNA expression in a control cell wherein the control cell comprises two copies of wild-type SNCA gene. The SNCA mRNA expression in the cell may be decreased compared to SNCA mRNA expression in a control cell wherein the control cell comprises two copies of wild-type SNCA gene. The SNCA mRNA expression in the cell may be decreased by about 50% compared to SNCA mRNA expression in a control cell wherein the control cell comprises two copies of wild-type SNCA gene. The cell may be present in a cell culture. The iPSC line with one copy of functional SNCA gene may be used to derive a neuronal precursor cell line. The iPSC line with one copy of functional SNCA gene may be used to derive a neuronal cell line. The derived neuronal cell line may be used to derive a dopaminergic (DA) neuron. The isogenic iPSC line may be used as cellular tool for in vitro studies to understand the molecular mechanism of α-syn under expression and overexpression in the pathogenesis of PD.

iPSC Line with Zero Copies of Functional SNCA Gene

Disclosed herein, is isogenic iPSC line comprising zero copies of alpha-synuclein (SNCA) gene, wherein the cell line is produced from induced pluripotent stem cell with alpha-synuclein (SNCA) gene triplication. The induced pluripotent stem cell with alpha-synuclein (SNCA) gene triplication may be human-derived. The SNCA gene may be a functional SNCA gene. The SNCA gene may be a wild-type SNCA gene. The functional SNCA gene may be a SNCA gene that encodes a protein with wild-type functionality. The functional SNCA gene may be a SNCA gene that encodes a fully functional α-syn protein. The functional SNCA gene may be a wild-type SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may not maintain expression of pluripotency markers. The cell may maintain differentiation potential. The cell may have reduced differentiation potential compared to a control cell, wherein the control cell comprises two copies of wild-type SNCA gene. The cell may have almost no differentiation potential compared to a control cell, wherein the control cell comprises two copies of wild-type SNCA gene. The cell may have no differentiation potential compared to a control cell, wherein the control cell comprises two copies of wild-type SNCA gene. The morphology of the cell during initial specification may be comparable to a control cell comprising two copies of the wild-type SNCA gene. The SNCA mRNA expression in the cell may be decreased compared to SNCA mRNA expression in a control cell wherein the control cell comprises two copies of wild-type SNCA gene. The cell may have almost no SNCA mRNA expression compared to SNCA mRNA expression in a control cell wherein the control cell comprises two copies of wild-type SNCA gene. The cell may have almost no SNCA mRNA expression. The cell may have no SNCA mRNA expression. The cell may be present in a cell culture. The iPSC line with zero copies of functional SNCA gene may be used to derive a neuronal precursor cell line. The iPSC line with zero copies of functional SNCA gene may be used to derive a neuronal cell line. The derived neuronal cell line may be used to derive a dopaminergic (DA) neuron. The isogenic iPSC line may be used as cellular tool for in vitro studies to understand the molecular mechanism of α-syn under expression and overexpression in the pathogenesis of PD.

Method of Generating iPSC Lines with Different Functional Copy Numbers of the SNCA Gene

Disclosed herein, are isogenic iPSC lines produced from induced pluripotent stem cell with alpha-synuclein (SNCA) gene triplication. The isogenic iPSC lines may be produced by: (a) contacting the induced pluripotent stem cell with SNCA gene triplication with (i) a synthetic polynucleotide that targets a target sequence in one or more of the SNCA genes, and (ii) a genetically engineered vector comprising a gene which encodes a nucleic acid-guided nuclease; and (b) assessing the cell for copies of the SNCA gene. The induced pluripotent stem cell with SNCA gene triplication may be contacted with one or more synthetic polynucleotides that target a target sequence in one or more of the SNCA genes. The induced pluripotent stem cell with alpha-synuclein (SNCA) gene triplication may be human-derived. The one or more of the SNCA genes may be a functional SNCA gene. The one or more of the SNCA genes may be a wild-type SNCA gene. The functional SNCA gene may be a SNCA gene that encodes a protein with wild-type functionality. The functional SNCA gene may be a SNCA gene that encodes a fully functional protein. The functional SNCA gene may be a wild-type SNCA gene. The synthetic polynucleotide may be a guide nucleic acid. The guide nucleic acid may be a guide DNA. The guide nucleic acid may be a chimeric DNA/RNA hybrid. The guide nucleic acid may be a guide RNA (gRNA). The target sequence may be in exon 2 of the one or more SNCA genes. The target sequence may be in exon 3 of the one or more SNCA genes. The target sequence may be in exon 4 of the one or more SNCA genes. The target sequence may be in exon 5 of the one or more SNCA genes. The target sequence may comprise: 5′ GAGAAAACCAAACAGGGTG 3′, 5′ GGACTTTCAAAGGCCAAGG 3′, 5′ GCTGCTGAGAAAACCAAAC 3′, 5′ GCTTCTGCCACACCCTGTT 3′, or 5′ GCAGCCACAACTCCCTCCT 3′. The nuclease may introduce a double strand break in the target sequence in one or more the SNCA genes. The target sequence in the one or more SNCA genes may be modified by non-homologous end joining. The nucleic acid-guided nuclease may be a CRISPR nuclease. The CRISPR nuclease may be Cas9. The CRISPR nuclease may be Cpfl. The guide nucleic acid may be guide DNA and the target may be modified by Argonaute proteins. The cell may have three copies of a functional SNCA gene. The cell may have two copies of a functional SNCA gene. The cell may have one copy of a functional SNCA gene. The cell may have zero copies of a functional SNCA gene. The cell may have normal karyotype. The cell growth and maintenance may be comparable to a control cell comprising two copies of wild-type SNCA gene. The cell viability and survival may be comparable to a control cell comprising two copies of wild-type SNCA gene. The cell may maintain expression of pluripotency markers. The cell may maintain differentiation potential. The morphology of the cell during initial specification may be comparable to a control cell comprising two copies of the wild-type SNCA gene. The SNCA mRNA expression in the cell may be proportional to the copies of functional SNCA genes in the cell. The cell may be present in a cell culture. The isogenic iPSC line may be used as cellular tool for in vitro studies to understand the molecular mechanism of α-syn under expression and overexpression in the pathogenesis of PD.

### Methods of Treatment

Methods of treating and/or preventing a disorder (e.g., Parkinson's disease) in an individual by modifying SNCA gene expression are provided herein. The methods involve administering to the subject a composition comprising a gene editing system as disclosed herein, in an effective amount to treat or prevent the disorder. Also disclosed herein are methods to modify expression of SNCA gene in an induced pluripotent stem cell.

Disclosed herein, is a method of modifying expression of alpha-synuclein (SNCA) gene in an individual in need thereof, the method comprising: administering to the individual a composition comprising (i) at least one synthetic polynucleotide that targets a target sequence in one or more of the SNCA genes, and (ii) a nucleic acid-guided nuclease, wherein targeting the target sequence represses the transcription of one or more SNCA genes, thereby modifying expression of the SNCA gene in the individual. The individual may be predisposed to a neurodegenerative disease. The individual may have a neurodegenerative disease. The individual may or may not have started showing motor symptoms. The neurodegenerative disease may be Parkinson's disease, Parkinson's-related disease, or synucleinopathy. The individual may have prodromal PD. The individual may overexpress the SNCA gene. The individual may have more than two copies of a functional SNCA gene. The individual may have more than two copies of a wild-type SNCA gene. The individual may have three copies of a functional SNCA gene. The individual may have three copies of a wild-type SNCA gene. The individual may have four copies of a functional SNCA gene. The individual may have four copies of a wild-type SNCA gene. The functional SNCA gene may be a SNCA gene that encodes a protein with wild-type functionality. The functional SNCA gene may be a SNCA gene that encodes a fully functional protein. The functional SNCA gene may be a wild-type SNCA gene. The synthetic polynucleotide may be a guide nucleic acid. The guide nucleic acid may be a guide DNA. The guide nucleic acid may be a chimeric DNA/RNA hybrid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site (i.e. an area of -50 to +300 bp) of one or more SNCA genes. The target sequence may comprise a sequence disclosed in Table 3.

The nucleic acid-guided nuclease may be a CRISPR nuclease. The CRISPR nuclease may be Cas9 (e.g. bacterial Cas9). The bacterial Cas9 may be from Staphylococcus aureus. The bacterial Cas9 may be from Streptococcus pyogenes. The nucleic acid-guided nuclease may be catalytically inactive (e.g. dCas9). The transcription of one or more SNCA genes may be repressed by interfering with transcription initiation, transcription elongation, RNA polymerase binding, transcription factor binding, or any combination thereof. Repressing the transcription of one or more SNCA genes may be reversible. Repressing the transcription of one or more SNCA genes may decrease the expression of the SNCA gene in the individual. The decreased expression of the SNCA gene may be comparable to the expression of SNCA gene in a control cell. The modified expression of SNCA gene may be comparable to the expression of SNCA gene in a control cell. The control cell may comprise two copies of functional SNCA gene. The transcription of SNCA gene may be repressed by at least 50% compared to transcription of SNCA gene before administration of the composition. The transcription of SNCA gene may be repressed by at least 45% compared to transcription of SNCA gene before administration of the composition. The transcription of SNCA gene may be repressed by at least 40% compared to transcription of SNCA gene before administration of the composition. The transcription of SNCA gene may be repressed by at least 35% compared to transcription of SNCA gene before administration of the composition. The transcription of SNCA gene may be repressed by at least 30% compared to transcription of SNCA gene before administration of the composition. The transcription of SNCA gene may be repressed by at least 25% compared to transcription of SNCA gene before administration of the composition. The transcription of SNCA gene may be repressed by at least 20% compared to transcription of SNCA gene before administration of the composition. The transcription of SNCA gene may be repressed by about 50% compared to transcription of SNCA gene before administration of the composition. The transcription of SNCA gene may be repressed by about 45% compared to transcription of SNCA gene before administration of the composition. The transcription of SNCA gene may be repressed by about 40% compared to transcription of SNCA gene before administration of the composition. The transcription of SNCA gene may be repressed by about 35% compared to transcription of SNCA gene before administration of the composition. The transcription of SNCA gene may be repressed by about 30% compared to transcription of SNCA gene before administration of the composition. The transcription of SNCA gene may be repressed by about 25% compared to transcription of SNCA gene before administration of the composition. The transcription of SNCA gene may be repressed by about 20% compared to transcription of SNCA gene before administration of the composition.

Disclosed herein, is a method of treating a neurodegenerative disease in an individual in need thereof, the method comprising: administering to the individual a composition comprising (i) at least one synthetic polynucleotide that targets a target sequence in one or more of the SNCA genes, and (ii) a nucleic acid-guided nuclease, wherein the individual overexpresses SNCA gene, and wherein targeting the target sequence represses the transcription of one or more SNCA genes, thereby treating the individual. The neurodegenerative disease may be Parkinson's disease, Parkinson's-related disease, or synucleinopathy. The individual may have prodromal PD. The individual may or may not have started showing motor symptoms. The individual may overexpress the SNCA gene. The individual may have more than two copies of a functional SNCA gene. The individual may have more than two copies of a wild-type SNCA gene. The individual may have three copies of a functional SNCA gene. The individual may have three copies of a wild-type SNCA gene. The individual may have four copies of a functional SNCA gene. The individual may have four copies of a wild-type SNCA gene. The functional SNCA gene may be a SNCA gene that encodes a protein with wild-type functionality. The functional SNCA gene may be a SNCA gene that encodes a fully functional protein. The functional SNCA gene may be a wild-type SNCA gene. The synthetic polynucleotide may be a guide nucleic acid. The guide nucleic acid may be a guide DNA. The guide nucleic acid may be a chimeric DNA/RNA hybrid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site (i.e. an area of -50 to +300 bp) of one or more SNCA genes. The target sequence may comprise a sequence disclosed in Table 3. The nucleic acid-guided nuclease may be a CRISPR nuclease. The CRISPR nuclease may be Cas9 (e.g. bacterial Cas9). The bacterial Cas9 may be from Staphylococcus aureus. The bacterial Cas9 may be from Streptococcus pyogenes. The nucleic acid-guided nuclease may be catalytically inactive (e.g. dCas9). The transcription of one or more SNCA genes may be repressed by interfering with transcription initiation, transcription elongation, RNA polymerase binding, transcription factor binding, or any combination thereof. Repressing the transcription of one or more SNCA genes may be reversible. Repressing the transcription of one or more SNCA genes may decrease the expression of the SNCA gene in the individual. The decreased expression of the SNCA gene may be comparable to the expression of SNCA gene in a control cell. The modified expression of SNCA gene may be comparable to the expression of SNCA gene in a control cell. The control cell may comprise two copies of functional SNCA gene. The transcription of SNCA gene may be repressed by at least 50% compared to transcription of SNCA gene before administration of the composition. The transcription of SNCA gene may be repressed by at least 45% compared to transcription of SNCA gene before administration of the composition. The transcription of SNCA gene may be repressed by at least 40% compared to transcription of SNCA gene before administration of the composition. The transcription of SNCA gene may be repressed by at least 35% compared to transcription of SNCA gene before administration of the composition. The transcription of SNCA gene may be repressed by at least 30% compared to transcription of SNCA gene before administration of the composition. The transcription of SNCA gene may be repressed by at least 25% compared to transcription of SNCA gene before administration of the composition. The transcription of SNCA gene may be repressed by at least 20% compared to transcription of SNCA gene before administration of the composition. The transcription of SNCA gene may be repressed by about 50% compared to transcription of SNCA gene before administration of the composition. The transcription of SNCA gene may be repressed by about 45% compared to transcription of SNCA gene before administration of the composition. The transcription of SNCA gene may be repressed by about 40% compared to transcription of SNCA gene before administration of the composition. The transcription of SNCA gene may be repressed by about 35% compared to transcription of SNCA gene before administration of the composition. The transcription of SNCA gene may be repressed by about 30% compared to transcription of SNCA gene before administration of the composition. The transcription of SNCA gene may be repressed by about 25% compared to transcription of SNCA gene before administration of the composition. The transcription of SNCA gene may be repressed by about 20% compared to transcription of SNCA gene before administration of the composition.

Disclosed herein, is a method of measuring efficacy of a treatment for neurodegenerative disease in an individual overexpressing SNCA gene, the method comprising: (a) determining the copy number of SNCA gene in the individual; (b) contacting an isogenic induced pluripotent cell comprising a copy number of SNCA gene the same as the individual with a composition comprising (i) at least one synthetic polynucleotide that targets a target sequence in one or more SNCA genes, and (ii) a nucleic acid-guided nuclease; (c) detecting the response in the cell; and (d) comparing said response to control cells. The detected response may be change in cell viability, cellular chemistry, cellular function, mitochondrial function, cell aggregation, cell morphology, cellular protein aggregation, gene expression, cellular secretion, cellular uptake, or combinations thereof. The detected response may be detecting expression of one or more SNCA genes. The method may further comprise (e) adjusting the treatment to get a response comparable to the control cells. The method may further comprise (f) administering the composition with efficacy for treatment of the neurodegenerative to the individual. The neurodegenerative disease may be Parkinson's disease, Parkinson's-related disease, or synucleinopathy. The individual may have prodromal PD. The individual may or may not have started showing motor symptoms. The individual may overexpress the SNCA gene. The individual may have more than two copies of a functional SNCA gene. The individual may have more than two copies of a wild-type SNCA gene. The individual may have three copies of a functional SNCA gene. The individual may have three copies of a wild-type SNCA gene. The individual may have four copies of a functional SNCA gene. The individual may have four copies of a wild-type SNCA gene. The functional SNCA gene may be a SNCA gene that encodes a protein with wild-type functionality. The functional SNCA gene may be a SNCA gene that encodes a fully functional protein. The functional SNCA gene may be a wild-type SNCA gene. The synthetic polynucleotide may be a guide nucleic acid. The guide nucleic acid may be a guide DNA. The guide nucleic acid may be a chimeric DNA/RNA hybrid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site (i.e. an area of -50 to +300 bp) of one or more SNCA genes. The target sequence may comprise a sequence disclosed in Table 3.The nucleic acid-guided nuclease may be a CRISPR nuclease. The CRISPR nuclease may be Cas9 (e.g. bacterial Cas9). The bacterial Cas9 may be from Staphylococcus aureus. The bacterial Cas9 may be from Streptococcus pyogenes. The nucleic acid-guided nuclease may be catalytically inactive (e.g. dCas9). The transcription of one or more SNCA genes may be repressed by interfering with transcription initiation, transcription elongation, RNA polymerase binding, transcription factor binding, or any combination thereof. Repressing the transcription of one or more SNCA genes may be reversible. Repressing the transcription of one or more SNCA genes may decrease the expression of the SNCA gene in the individual. The decreased expression of the SNCA gene may be comparable to the expression of SNCA gene in a control cell. The modified expression of SNCA gene may be comparable to the expression of SNCA gene in a control cell. The control cell may be an isogenic induced pluripotent cell comprising a copy number of SNCA gene the same as the individual without contact with the composition, or an isogenic induced pluripotent cell comprising two functional copies of SNCA gene without contact with the composition, or both. The transcription of SNCA gene may be repressed by at least 50% compared to transcription of SNCA gene before administration of the composition. The transcription of SNCA gene may be repressed by at least 45% compared to transcription of SNCA gene before administration of the composition. The transcription of SNCA gene may be repressed by at least 40% compared to transcription of SNCA gene before administration of the composition. The transcription of SNCA gene may be repressed by at least 35% compared to transcription of SNCA gene before administration of the composition. The transcription of SNCA gene may be repressed by at least 30% compared to transcription of SNCA gene before administration of the composition. The transcription of SNCA gene may be repressed by at least 25% compared to transcription of SNCA gene before administration of the composition. The transcription of SNCA gene may be repressed by at least 20% compared to transcription of SNCA gene before administration of the composition. The transcription of SNCA gene may be repressed by about 50% compared to transcription of SNCA gene before administration of the composition. The transcription of SNCA gene may be repressed by about 45% compared to transcription of SNCA gene before administration of the composition. The transcription of SNCA gene may be repressed by about 40% compared to transcription of SNCA gene before administration of the composition. The transcription of SNCA gene may be repressed by about 35% compared to transcription of SNCA gene before administration of the composition. The transcription of SNCA gene may be repressed by about 30% compared to transcription of SNCA gene before administration of the composition. The transcription of SNCA gene may be repressed by about 25% compared to transcription of SNCA gene before administration of the composition. The transcription of SNCA gene may be repressed by about 20% compared to transcription of SNCA gene before administration of the composition.

### Pharmaceutical Compositions

Disclosed herein, is a pharmaceutical composition for treatment of a neurodegenerative disease in an individual in need thereof, comprising (i) at least one synthetic polynucleotide that targets a target sequence in one or more of SNCA genes, and (ii) a nucleic acid-guided nuclease; and a pharmaceutically-acceptable excipient, wherein the composition has efficacy in the treatment of the neurodegenerative disease, wherein said efficacy is measured according to the method disclosed herein. The neurodegenerative disease may be Parkinson's disease, Parkinson's-related disease, or synucleinopathy. The individual may have prodromal PD. The individual may or may not have started showing motor symptoms. The individual may overexpress the SNCA gene. The individual may have more than two copies of a functional SNCA gene. The individual may have more than two copies of a wild-type SNCA gene. The individual may have three copies of a functional SNCA gene. The individual may have three copies of a wild-type SNCA gene. The individual may have four copies of a functional SNCA gene. The individual may have four copies of a wild-type SNCA gene. The functional SNCA gene may be a SNCA gene that encodes a protein with wild-type functionality. The functional SNCA gene may be a SNCA gene that encodes a fully functional protein. The functional SNCA gene may be a wild-type SNCA gene. The synthetic polynucleotide may be a guide nucleic acid. The guide nucleic acid may be a guide DNA. The guide nucleic acid may be a chimeric DNA/RNA hybrid. The guide nucleic acid may be a guide RNA (gRNA). The synthetic polynucleotide may comprise a transcriptional start site of one or more SNCA genes. The target sequence may be in the promoter region of one or more SNCA genes. The target sequence may be proximal to a transcriptional start site (i.e. an area of -50 to +300 bp) of one or more SNCA genes. The target sequence may comprise a sequence disclosed in Table 3.The nucleic acid-guided nuclease may be a CRISPR nuclease. The CRISPR nuclease may be Cas9 (e.g. bacterial Cas9). The bacterial Cas9 may be from Staphylococcus aureus. The bacterial Cas9 may be from Streptococcus pyogenes. The nucleic acid-guided nuclease may be catalytically inactive (e.g. dCas9).

The compositions described herein can be administered in a variety of different ways. The compositions may be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds may be achieved in various ways, including intraparenchymal, intracerebroventricular, intracranial, oral, buccal, rectal, parenteral, intraperitoneal, intravenous, intramuscular, topical, subcutaneous, subdermal, intradermal, transdermal, intrathecal (cisternal or lumbar), nasal, intracheal, etc., administration. The composition may be systemic after administration or may be localized by the use of regional administration, intramural administration, or use of an implant that acts to retain the active dose at the site of implantation. For example, the composition may be intracranially administered using, e.g., an osmotic pump and microcatheter or other neurosurgical device to deliver the composition to selected regions of the brain under singular, repeated or chronic delivery regimens. In some aspects, composition may cross and or even readily pass through the blood-brain barrier, which permits, e.g., oral, parenteral or intravenous administration. Alternatively, the composition may be modified or otherwise altered so that it can cross or be transported across the blood brain barrier. Many strategies known in the art are available for molecules crossing the blood-brain barrier, including but not limited to, increasing the hydrophobic nature of a molecule; introducing the molecule as a conjugate to a carrier, such as transferring, targeted to a receptor in the blood-brain barrier, or to docosahexaenoic acid etc. In another aspect, a composition is administered via the standard procedure of drilling a small hole in the skull to administration. The composition may be administered intracranially or, for example, intraventricularly. Osmotic disruption of the blood-brain barrier may be used to effect delivery of composition to the brain (Nilaver et al., Proc. Natl. Acad. Sci. USA 92:9829-9833 (1995)). A composition may be administered in a liposome targeted to the blood-brain barrier. Administration of pharmaceutical compositions in liposomes is known (see Langer, Science 249:1527-1533 (1990); Treat et al., in Liposomes in the Therapy of infectious Disease and Cancer, Lopez-Berestein and Fidler (eds.), Liss, New York, pp. pp. 317-327 and 353-365 (1989). All of such methods are envisioned herein.

Therapeutic compositions may include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation may include other carriers, adjuvants, or non-toxic, nontherapeutic, nonimmunogenic stabilizers, excipients and the like. The compositions may also include additional substances to approximate physiological conditions, such as pH adjusting and buffering agents, toxicity adjusting agents, wetting agents, and detergents.

Further guidance regarding formulations that are suitable for various types of administration may be found in Remington's Pharmaceutical Sciences, Mace Publishing Company, Philadelphia, Pa., 17th ed. (1985). For a brief review of methods for drug delivery, see, Langer, Science 249:1527-1533 (1990).

The compositions disclosed herein may be administered for prophylactic and/or therapeutic treatments. Toxicity and therapeutic efficacy of the compositions may be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are used in some embodiments.

The data obtained from cell culture and/or animal studies may be used in formulating a range of dosages for humans. The dosage of the active ingredient typically lines within a range of circulating concentrations that include the ED50 with low toxicity. The dosage may vary within this range depending upon the dosage form employed and the route of administration utilized.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The effective amount of a therapeutic composition to be given to a particular patient may depend on a variety of factors, several of which will be different from patient to patient. A competent clinician will be able to determine an effective amount of a therapeutic agent to administer to a patient. Dosage of the composition will depend on the treatment, route of administration, the nature of the therapeutics, sensitivity of the patient to the therapeutics, etc. Utilizing LD50 animal data, and other information, a clinician can determine the maximum safe dose for an individual, depending on the route of administration. Utilizing ordinary skill, the competent clinician will be able to optimize the dosage of a particular therapeutic composition in the course of routine clinical trials. The compositions may be administered to the subject in a series of more than one administration. For therapeutic compositions, regular periodic administration will sometimes be required, or may be desirable. Therapeutic regimens will vary with the compositions and from patient to patient, e.g., compositions may be taken for extended periods of time on a daily or semi-daily basis, or may be administered for more defined time courses, e.g., one, two three or more days, one or more weeks, one or more months, etc., taken daily, semi-daily, semi-weekly, weekly, etc.

A pharmaceutically or therapeutically effective amount of the composition is delivered to the individual in need thereof. The precise effective amount will vary from subject to subject and will depend upon the age, the individual's size and health, the nature and extent of the condition being treated, recommendations of the treating physician, and the therapeutics or combination of therapeutics selected for administration. Thus, the effective amount for a given situation may be determined by routine experimentation. The individual may be administered in as many doses as is required to reduce and/or alleviate the signs, symptoms, or causes of the disorder in question (e.g. Parkinson's disease), or bring about any other desired alteration of a biological system.

### EXAMPLES

### Example 1

### Generation of Isogenic iPSC Lines with Different Wild-Type Copies of the SNCA Gene

Multiple clonal iPSC lines from skin cells of a PD patient carrying a triplication of the SNCA gene have been reprogrammed using a retroviral system with four factors encoding OCT4, KLF4, SOX2, and cMYC9. All iPSC lines were characterized for pluripotency, differentiation potential, silencing of transgenes, and have a normal karyotype.

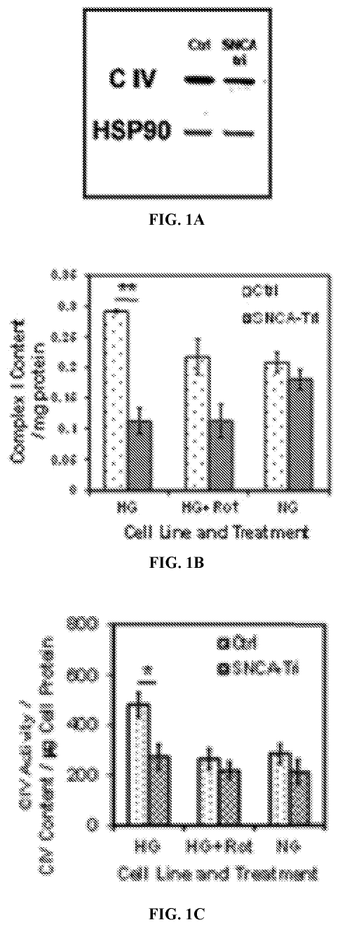

The function of mitochondrial respiratory chain, and particularly mitochondrial Complex I has been shown to be affected in PD, and fibroblasts with the SNCA gene triplication have altered mitochondrial complex I function. Preliminary results also show that these defects are present in there from derived NPCs, which also presented with reduced complex IV protein levels and reduced complex IV function (FIG. 1A-FIG. 1C). In addition, SNCA triplication cell lines exhibit reduced mitochondrial protein import function.

### Example 2

### Generation of Isogenic iPSC Lines with Different Functional Copy Numbers of the SNCA Gene

iPSC clones from a donor with an SNCA gene triplication (descendant from the Iowa kindred) were used to engineer and genetically characterize a panel of patient-derived isogenic induced pluripotent stem cells lines that carry different functional copies of the SNCA gene, ranging from four to zero functional gene copies). The set of iPSC lines may be used as a model system for further functional studies of the physiological role of α-syn in human neurons.

Induced pluripotent stem cell (iPSC) culture and maintenance: iPSCs were cultured on Geltrex with manual passaging every 6-7 days. Essential 8 media was changed daily.

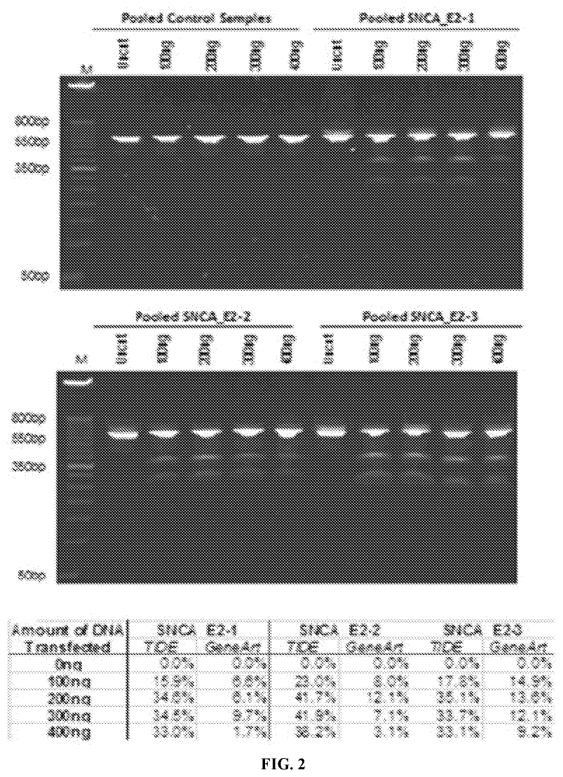

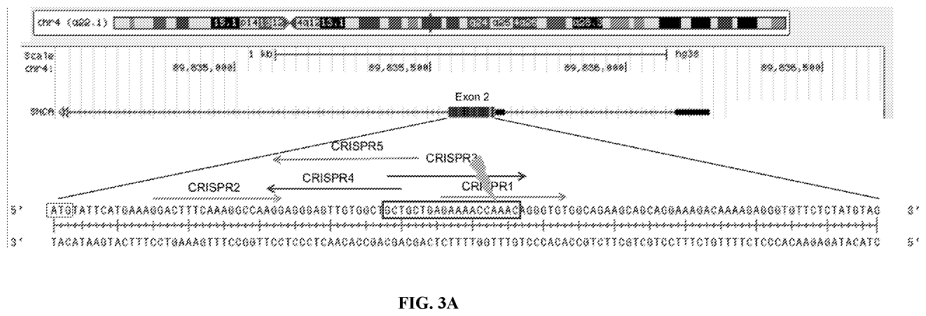

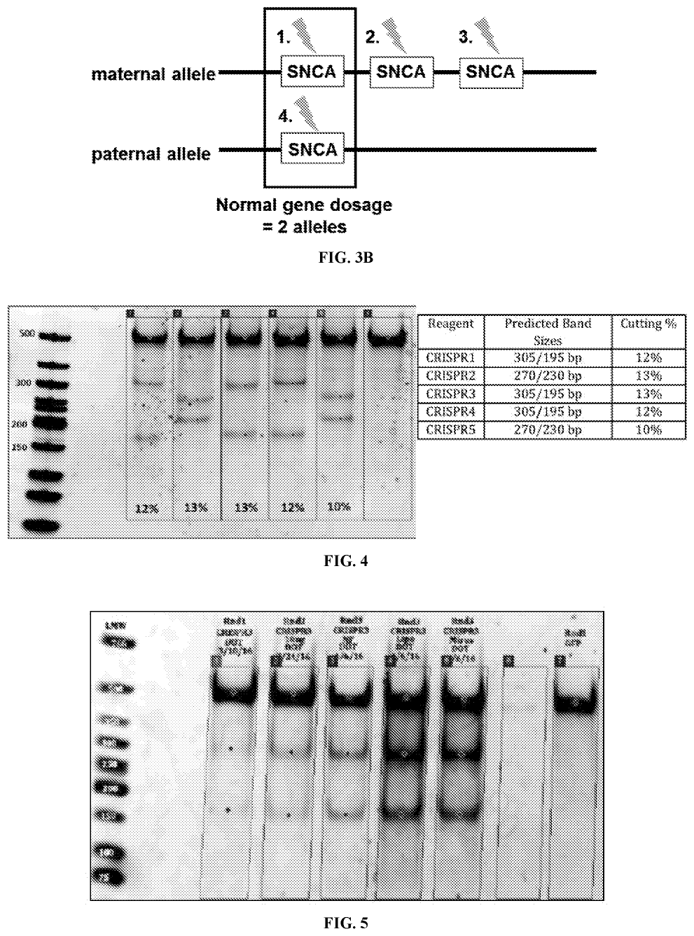

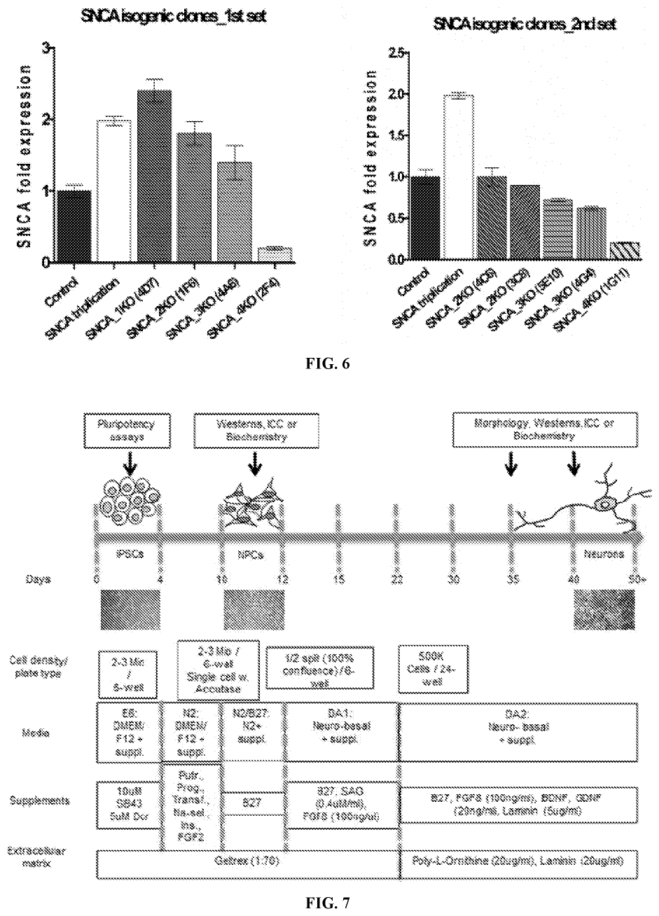

CRISPR reagent design-build, transfection and screening: Cells were adapted to single-cell passaging techniques required for efficient gene editing. Several small guide (sg) RNAs that specifically target the SNCA gene exons 2 through 5 were experimentally determined and their target specificity in HEK293 cells was validated (FIG. 2). One sgRNA with high cutting efficiency (SNCA_E2_2, about 40%) was identified. Five gRNAs (reagents) were designed and built to exon 2 of SNCA (Table 1). HEK293T were transfected with each reagent to assess cutting at the target locus via Cel-1 assay. Transfection with the nucleases in iPSCs was performed 3 times sequentially (over 6 weeks).

TABLE 1
A panel of tested CRISPR guide RNAs
used to knock-out SNCA.

| sgRNA ID | Target Sequence |
|---|---|
| CRISPR1 | 5′ GAGAAAACCAAACAGGGTG 3′ |
| CRISPR2 | 5′ GGACTTTCAAAGGCCAAGG 3′ |
| CRISPR3 | 5′ GCTGCTGAGAAAACCAAAC 3′ |
| CRISPR4 | 5′ GCTTCTGCCACACCCTGTT 3′ |
| CRISPR5 | 5′ GCAGCCACAACTCCCTCCT 3′ |

Transfected pools were screened for the presence of the indels indicating repair via NHEJ at the cut-sites. Cel-1 assay was performed to assess the level of cutting at the sites after each round of transfection. The consolidated clones were initially assessed for knockout alleles utilizing droplet digital PCR. The sequence confirmed clones were further expanded to make the final cell banks for future experiment.

TABLE 2
CRISPR-modified SNCA alleles in human iPSC

| Clone # | KO Status | Allele Status | Notes |
|---|---|---|---|
| 1 | 4 KO | del4/del5 (no wt) | Internal clone 1G11; no evidence of WT in 55 sequences |
| 2 | 4 KO | del5/del26/del8/ins14 | Internal clone 2F4; see 4 KO alleles |
| 3 | 4 KO | del2/del4/del5 (no wt) | Internal clone 3B2; no evidence of WT in 50 sequences |
| 4 | 3 KO | wt/ins1/del5/ins8 | Internal clone 4A6; see all 4 alleles |
| 5 | 3 KO | wt/del5/del19/ins1 | Internal clone 4G4; see all 4 alleles |
| 6 | 3 KO | wt/del5/del19/ins13 | Internal clone 5E10; se all 4 alleles |
| 7 | 2 KO | wt/del5/del17 | Internal clone 1F6; see 3 alleles (assumes 2 WT; |
| | | | matches ddPCR) |
| 8 | 2 KO | wt/del4/del5 | Internal clone 3C8; see 3 alleles (assumes 2 WT; matches |
| | | | ddPCR) |
| 9 | 2 KO | wt/del4/del34 | Internal clone 4C6; see 3 alleles (assumes 2 WT; matches |
| | | | ddPCR) |
| 10 | n/a | wt/del5/del68/del34/del2 | Internal clone 4B6; NOT EXPANDED; apparent mixed |
| | | | clone |
| 11 | 1 KO | wt/del5 | Internal clone 4D7; see 2 alleles (assumes 3 WT; |
| | | | matches ddPCR) |

Clonally expanded gene-edited iPSC lines are screened with PCR-based heteroduplex-detecting assays. Alternatively, gene-edited iPSCs clones are screened using hetero-duplex analysis of PCR-amplified target sites by denaturing high-performance liquid chromatography (DHPLC), e.g. Transgenomics® WAVE system or bioinformatics approach using Sanger sequence information to detect small insertions or deletions by decomposition analysis (TIDE). Clones that show targeted disruption of the SNCA gene in the initial screen are selected and further characterized by the following strategies: 1) By cloning of SNCA target location-spanning PCR fragments and Sanger sequencing of individual clones; 2) By quantitative RT-PCR analysis of SNCA gene transcripts, to detect differential expression levels of functional SNCA mRNAs; 3) By analyzing α-syn protein levels by immunoblotting.

All positive clones with the desired functional copy number of the SNCA gene (zero copies/knock-out, 1, 2, 3, and 4 functional copies) are characterized for maintenance of pluripotency, differentiation potential, and normal karyotype.

If the targeting with a single gRNA does not result in disturbance of all alleles in an iPSC clone, either cumulative or sequentially sgRNAs targeting to other exons of the SNCA gene is employed. Additional sgRNAs to exons 3 to 5 are designed and cloned.

### Example 3

### Testing the Effect of α-Syn on Structure and iPSC-Differentiated Neurons with Different Functional Gene Copies of the SNCA Gene

A panel of assays has been developed to characterize mitochondrial function and bioenergetics and is applied to the derived SNCA CRISPR/Cas9-derived iPS neuronal cultures. The effect of different SNCA functional copies on steady state levels and dynamics of subcellular membrane systems (mitochondria, lysosomes, endoplasmic reticulum (ER), and particular the mitochondrial respiratory chain assembly and function is determined. α-syn protein modification and changes in level of α-syn interacting proteins is also studied.

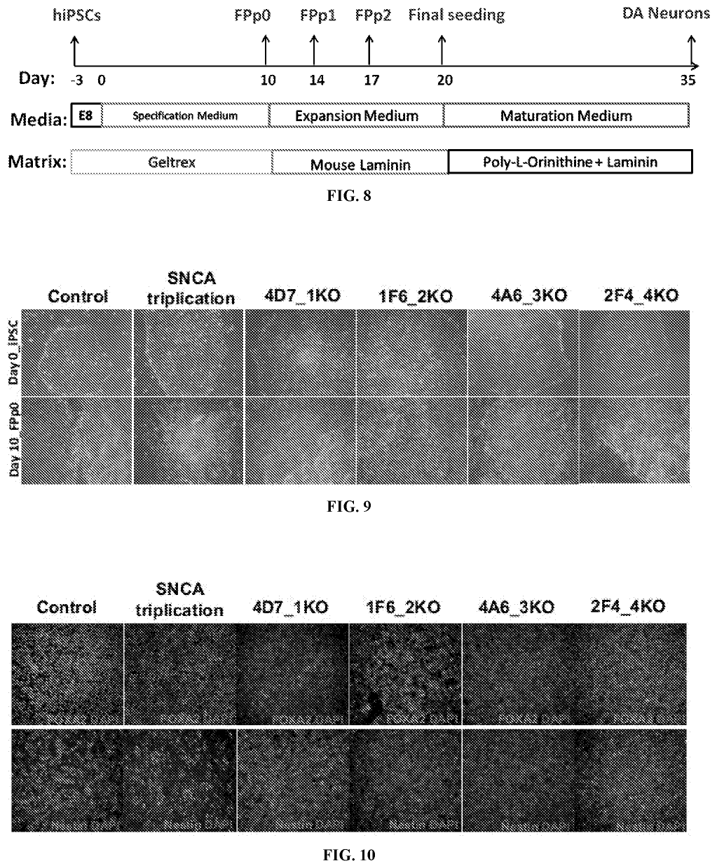

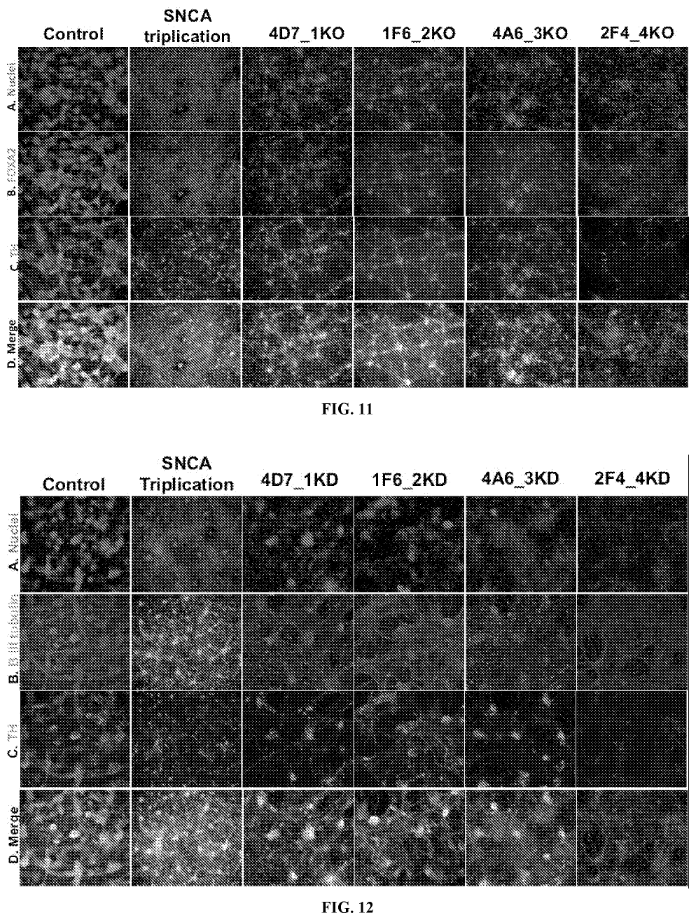

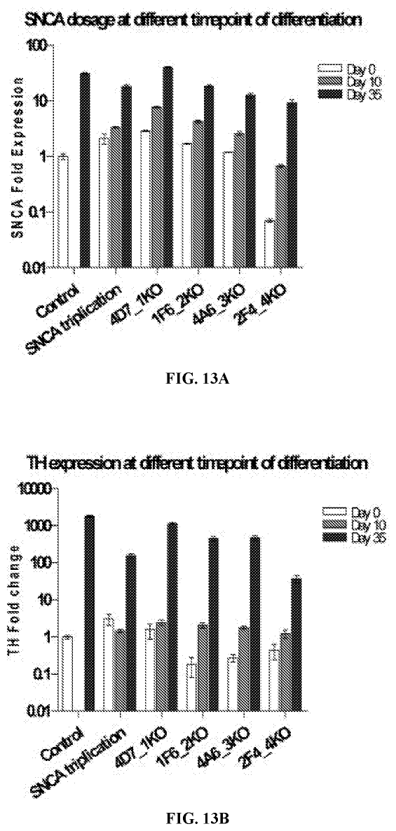

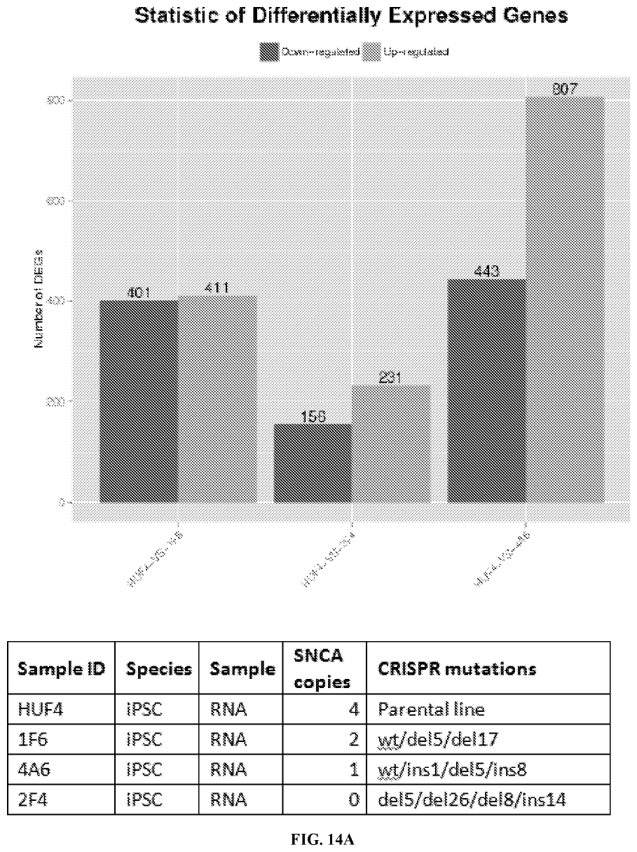

Gene-edited iPSCs are differentiated into neurons using a direct differentiation protocol as shown in FIG. 7 or FIG. 8, allowing for direct differentiation of iPSCs into dopaminergic (DA) neurons without embryoid body formation. This protocol allows for a faster and less labor intensive differentiation of iPSCs into neurons, while still preserving the neuronal precursor cell (NPC) stage of development. At least three clones of each genotype were differentiated for later analyses.

PSC midbrain dopaminergic (DA) neuron differentiation: Differentiation from iPSCs to DA neurons were achieved using PSC midbrain DA differentiation kit (ThermoFisher, A3147701). iPSCs were cultured for 10 days with specification media to generate floor plate progenitors (FPps). FPps were expanded for 10 more days with expansion media to be either cryopreserved or further maturation. The last 15 days of the differentiation process, FPps were developed into functional DA neurons with maturation media.

During differentiation, neurons are monitored for viability. cl Example 4

### Further Testing of the iPSC-Differentiated Neurons with Different Functional Gene Copies of the SNCA Gene

Successfully differentiated neuronal lines are analyzed using the following work-flow to maximize utilization of the generated neurons:

Live cell imaging: Morphological or developmental differences (such as the number of generated neurons/genotype, neurite outgrowth) in gene-edited neuronal lines are assessed. As abnormal α-syn levels have been associated with altered transport and interaction of organelles, neurons are transduced with baculoviral vectors encoding fluorescent proteins to investigate protein biosynthesis capability and membrane transport in these live neurons by imaging. Time-resolved expression of organelle-specific targeted fluorescent proteins (Living Colors™) in the nucleus, lysosomes, peroxisomes and mitochondria using Nikon T1 automated microscope is monitored and compared with environmental controls for time resolved microscopy.

Immunocytochemistry: Neuronal immunocytochemistry panel is employed on the engineered neurons for early and mature neuronal markers as well as dopaminergic neurons (Nestin, B3 tubulin, FOXA2, LMX1A, tyrosine hydroxylase). Additionally, these cells are stained for α-syn, 14-3-3 proteins and LRRK2. These stains are conducted on the fluorescent protein-transduced cell lines, thus gaining additional information about co-localization of the antibody targets with cellular organelles.

Cells were fixed at day10 and day35 in 4% PFA and permeabilized with 0.3% triton X-100 in PBS for 5 minutes (except cells stained with tyrosine hydroxylase (TH, Millipore, ab152) and β-III-Tubulin (TUJ1, Covance, MMS-435P)) antibodies, blocked with 10% goat serum for 1 hour at RT, and incubated with primary antibodies (at day10, with FOXA2 (ThermoFisher, A29515) and NESTIN (Milipore, MAB5326)) for overnight at 4C. Indirect immunofluorescence staining was performed with Alexa fluor 488 and 555 conjugated H+L antibodies. Fluorescent images were captured on an Nikon Eclipse Ti inverted fluorescence microscope and analyzed with ANDOR Zyla software.

Taqman gene expression analysis: Total RNA was collected using Qiagen Rneasy Minikit from Trizol treated cells at day0, day10, and day35. cDNA was synthetized using the iScript™ cDNA Synthesis Kit. Taqman probes FAM-MGB labeled SNCA, FAM-MGB labeled TH and for normalization VIC-MGB_PL labeled ACTB were used for relative expression analysis. Relative expression levels were calculated with subsequent ACT values that were analyzed using CFX software.

Biochemical assays and immunoblotting: Lysates are prepared from these engineered neurons for biochemical assays and immunoblotting. Function of mitochondrial respiratory chain complex I has been repeatedly shown to be affected in PD. For biochemical analysis, mitochondrial respiratory chain complexes I and IV is immune-precipitated from neuronal cell lysates and analyzed by microplate ELISA for content and activity of these two complexes. If biological material available for biochemistry from differentiated neurons is limiting, these assays are alternatively performed on the intermediate-stage NPCs, as some of the phenotypical and pathological changes observed in SNCA triplication neurons may be observed at the NPC stage.

Western analysis: Neuronal cell lysates are used for Western analysis of selected nuclear and mitochondria-encoded proteins of complex Ito assess possible assembly or chaperone defects. Additional Western analysis is focused on mechanisms connecting expression levels as well as phosphorylation status of α-syn and LRRK2, as they have been suggested to play a role in aggregation and toxicity of α-syn. The differential phosphorylation of α-syn and LRRK2 may hint on the functional relationship between LRKK2 and a-syn. Protein content of α-syn interacting proteins, (such as 14-3-3 chaperone proteins17, 18 and the α-syn interacting Polo-Like Kinase 2, PLK219) is investigated. Finally, distribution and amount of the mitochondrial protein import complex TOM40, which is affected by overexpression of a-syn, is assessed.

In case of subtle and non-significant differences in the functional assays comparing single copy differences of the SNCA gene in engineered neurons, these neurons are challenged with neurotoxins that have been shown to contribute to development of PD pathology. Treatment strategies for iPSC derived neurons with both the dopaminergic neurotoxin MPTP and the mitochondrial complex I specific toxin rotenone have been established. For both of these toxins alteration in cellular function in SNCA triplication NPCs is seen.

### Example 5

### Characterization of Isogenic iPSC Lines with Different Functional Copy Numbers of the SNCA Gene Via Ttranscriptome Sequencing

RNA sequencing: Total RNA extraction and DNase I treatment was performed on the isogenic iPSC lines, after which magnetic beads with Oligo (dT) were used to isolate mRNA. Mixed with the fragmentation buffer, the mRNA was fragmented into short fragments. Then cDNA was synthesized using the mRNA fragments as templates. Short fragments were purified and resolved with EB buffer for end reparation and single nucleotide A (adenine) addition. After that, the short fragments were connected with adapters. After agarose gel electrophoresis, the suitable fragments were selected for the PCR amplification as templates. During the QC steps, Agilent 2100 Bioanaylzer and ABI StepOnePlus Real-Time PCR System were used in quantification and qualification of the sample library. Lastly, the library was sequenced using Illumina HiSeq™ 2000 or other sequencer when necessary.

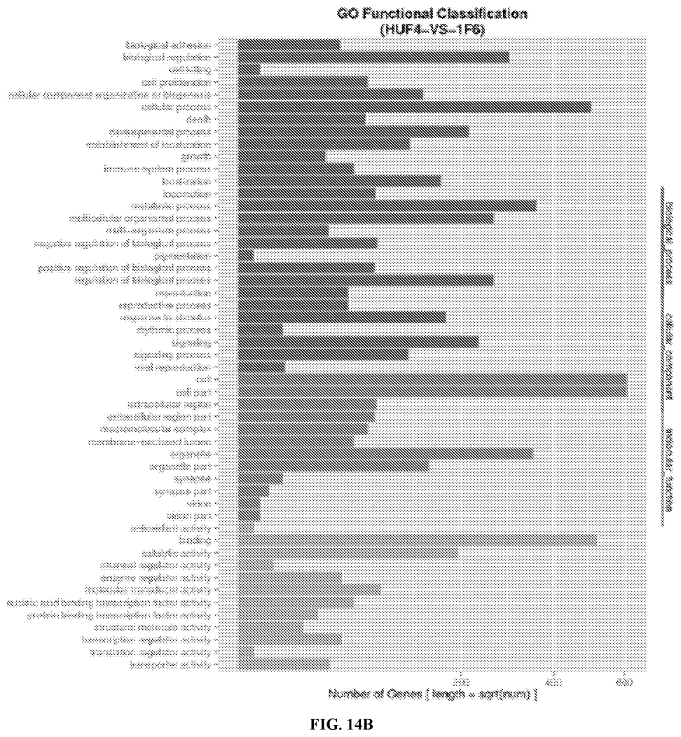

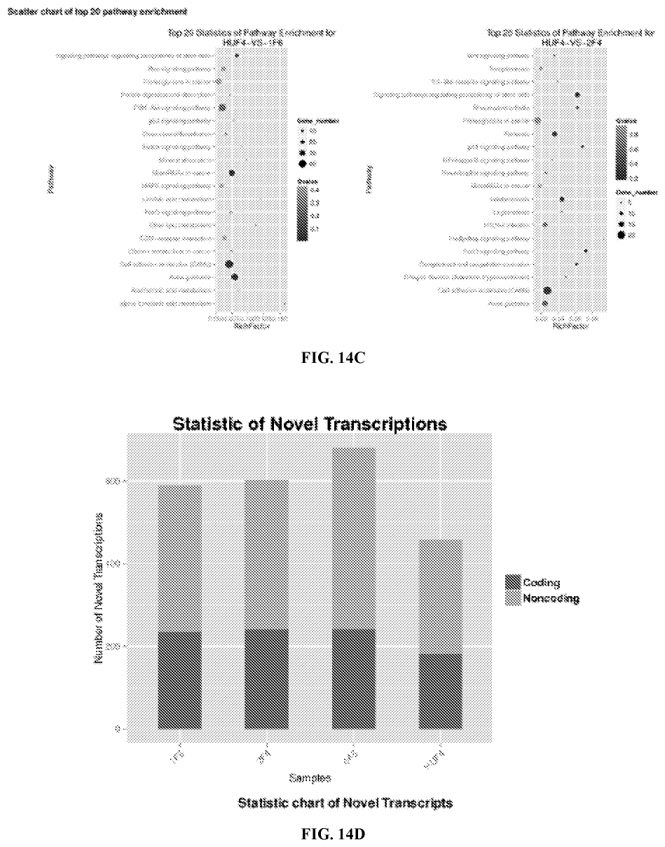

Bioinformatics analysis: Primary sequencing data produced by Illumina HiSeq™ 2000, called as raw reads, were subjected to quality control (QC) that determine if a resequencing step is needed. After QC, raw reads were filtered into clean reads which are aligned to the reference sequences. QC of alignment was performed to determine if resequencing is needed. The alignment data was utilized to calculate distribution of reads on reference genes and mapping ratio. When alignment result passed QC, downstream analysis including gene and isoform expression, deep analysis based on gene expression (PCA/correlation/screening differentially expressed genes and so on), exon expression, gene structure refinement, alternative splicing, novel transcript prediction and annotation, SNP detection, Indel detection, gene fusion was performed. Further, we also can perform deep analysis based on DEGs, including Gene Ontology (GO) enrichment analysis, Pathway enrichment analysis, cluster analysis, protein-protein interaction network analysis and finding transcriptor factor was also performed. FIG. 14A-FIG. 14D exemplify the results obtained.

### Example 6

### Optimization of sgRNA SNCA Promoter Candidates that Downregulate SNCA Gene Expression in HEK293T Cells

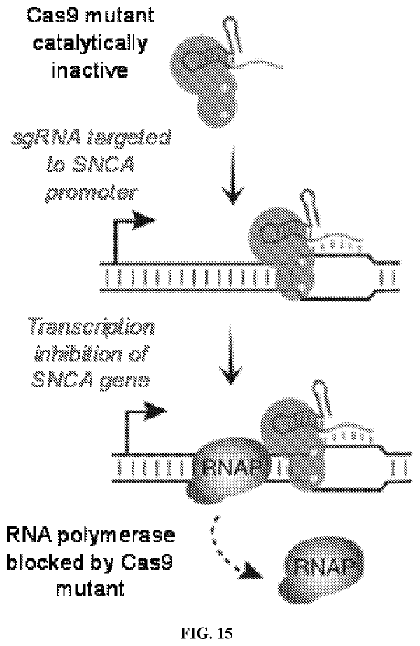

An advanced method of the CRISPR technology for gene regulation, named CRISPR interference is applied develop new therapeutic candidates to stop neurodegeneration in PD. Targeting of catalytically inactive Cas9 protein (dCas9) to the promoter region of a gene may sterically hinder the RNA polymerase binding and also impairs transcription initiation, leading to downregulation of gene expression which may be titrated depending on the design of the small guide RNA of the chimera RNA molecule (FIG. 15).

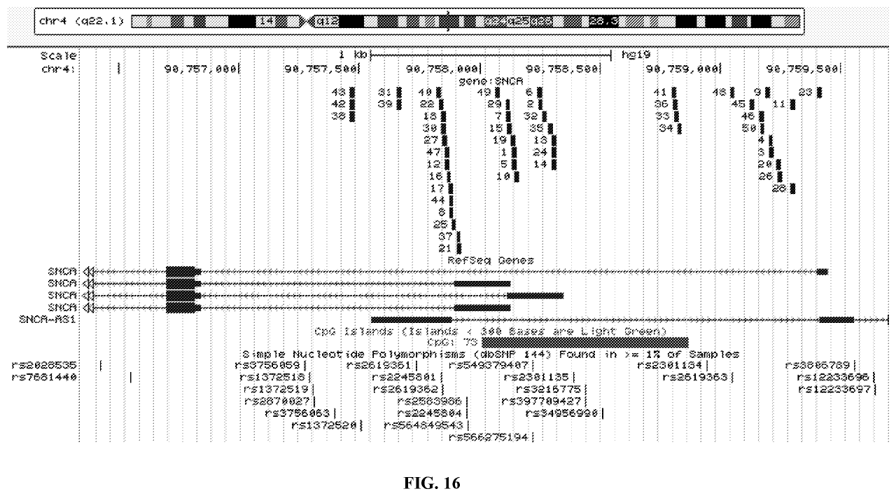

A toolset of synthetic guide RNAs (sgRNA) directed to the human SNCA promoter is optimized by first testing known documented sgRNAs, for e.g. an sgRNA pair of the template and non-template strand of the SNCA promoter that mediates repression of SNCA gene expression by ˜50%. Second, additional sgRNAs are tested using refined in silico design tools with functionally confirmed promoter by Fantom/CAGE annotation (FIG. 16). Efficient gene repression may be achieved when the sgRNA targets an area of -50 to +300 bps around the transcriptional start site of a promoter. The design of most efficient sgRNAs using the functional Fantom5/CAGE promoter annotation showed more efficient silencing as compared to GENCODE V19, UCSC genes, or RefSeq genes. A panel of designed CRISPR guide RNAs used to target the three transcription start sites of SNCA is shown in Table 3. 33 sgRNAs were designed to cover 3 kb of SNCA promoter region using CRISPOR, and the S. aureus PAM sequence.

TABLE 3
A panel of designed CRISPR guide RNAs used
to target SNCA promoter region.
Proximal to Transcription Start Site 1 (TSS1)

| | |
|---|---|
| 849R | TTCCAGTGCTTAACTAATCTT |
| 845F | GAAAGCCTTTGCTTTCTGTGC |
| 792F | ACAGCTGTTCCTGGATCACAC |
| 645F | ACTTCTGATTCTCGTTGCCCT |
| 564R | CAGAAGGGGCTGAAGAAGAAA |
| 532R | GGTCCGGTAGGCTAAATCACG |

Proximal to Transcription Start Site 2.1 (TSS2.1)

| | |
|---|---|
| 821F | ATGGGGATGGGGCAGGGGGCG |
| 802F | CTCCTCGTCCCTATCTCGGAT |
| 836F | TTGCGCGGCCAGGCAGGCGGC |
| 571F | ACGCTCTCGGAGGGGCCGGGC |

Proximal to Transcription Start Site 2.2 (TSS2.2)

| | |
|---|---|
| 479R | CACCCTCGTGAGCGGAGAACT |
| 453R | TGGCCATTCGACGACAGGTTA |
| 469F | GGCAAACCCGCTAACCTGTCG |
| 438F | GAGCCGGCGACGCGAGGCTGG |
| 382R | CTCCTCTGGGGACAGTCCCCC |
| 317R | CTTTCCTATTAAATATTATTT |
| 267R | AAGAGAGAGGCGGGGAGGAGT |
| 228R | GAGGGACTCAGGTAAGTACCT |
| 244F | TTTAGATCCACAGGTACTTAC |
| 178R | CTGGAGAACGCCGGATGGGAG |
| 202F | CGTCTCCCATCCGGCGTTCTC |
| 155R | GAATGGTCGTGGGCACCGGGA |

Proximal to Transcription Start Site 3 (TSS3)

| | |
|---|---|
| 738R | GCGCGGGGTTGGAGACGGCCC |
| 716R | CGAGTGTGAGCGGCGCCTGCT |
| 696F | TCAGGGTAGATAGCTGAGGGC |
| 626R | TGCCTGAGTTTGAACCACACC |
| 629F | CAACAGAACTAACTGCTCACT |
| 552R | CCTTCTTCTGGGATTCATGTT |
| 539F | GAAGCATGGAAGCTGGAGGCT |
| 510F | CGGGGTCTAAAGGGTAACAGT |
| 417F | AAATGCAGTAATAACAACTCA |
| 681R | GAGGGCGGGGGTGGATGTTGG |
| 155F | TTGGCCTTTGAAAGTCCTTTC |

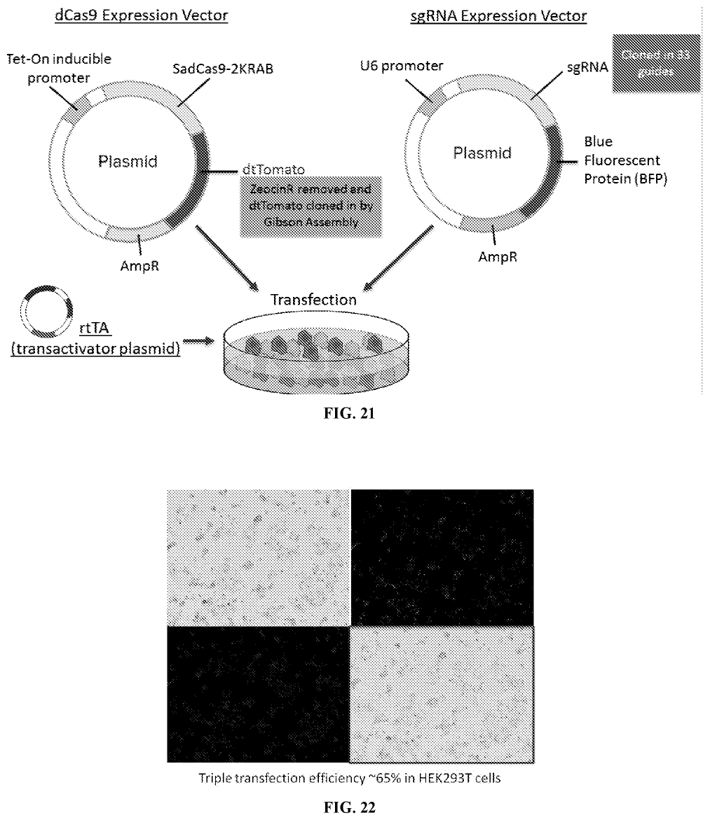

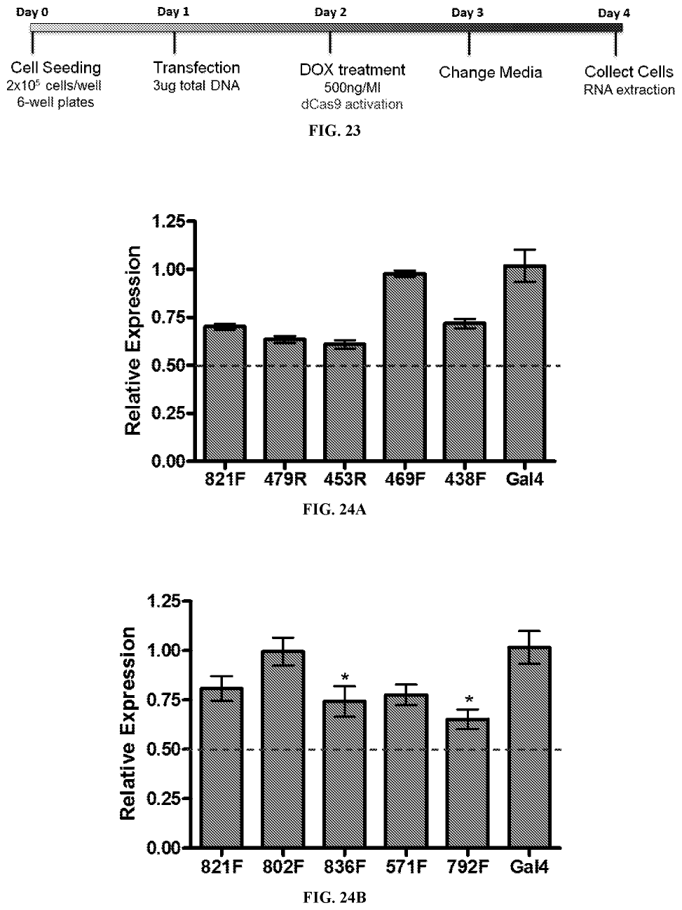

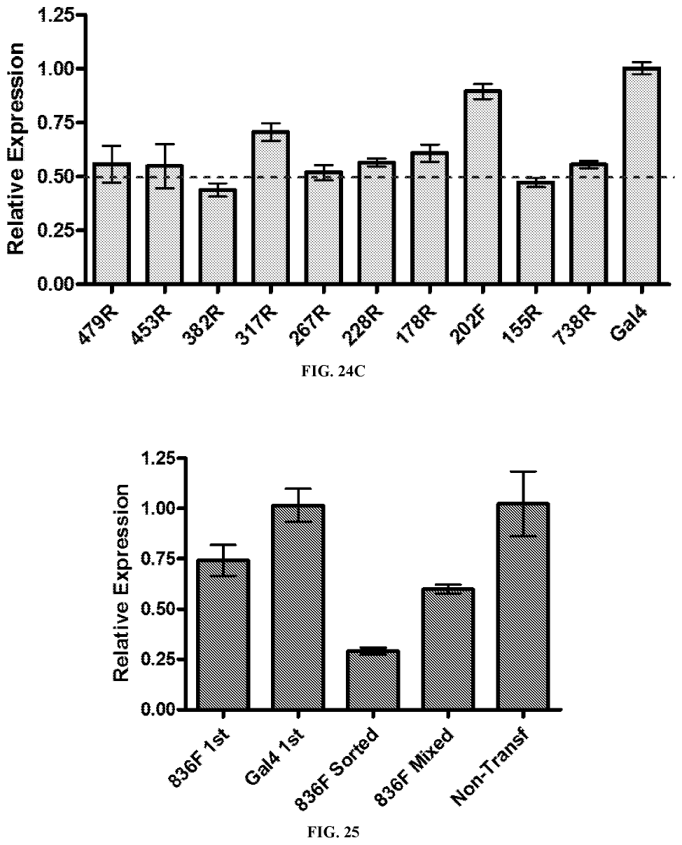

These sgRNAs were tested in HEK293 cell line. Doxycycline-inducible dcas9-KRAB expression vector with dtTomato selection marker was used. Each of the 33 sgRNAs was cloned in a separate expression vector with a blue fluorescent protein (BFP) selection marker (FIG. 21). Both dCas9 and sgRNA expression vectors were transfected into HEK293 cells along with rtTA (transactivator plasmid) and stained. Double positive cells contain both dCas9 (red) and sgRNA (blue) expression vectors and showed triple transfection efficiency of about 65% in HEK293T cells (FIG. 22).

HEK293T cells were transiently transfected with dCas9-KRAB, sgRNA and rtTA plasmids. 24 h post-transfection, 500 ng/mL DOX was added. Cells were collected 72 h post transfection and SNCA mRNA expression was determined. sgRNA candidates with >/=50% reduction in SNCA gene expression were identified (for example, 155R, 267R, 438F, and 836R). sgRNAs targeting sequences around the second transcriptional start site (TSS2) of SNCA showed more repressive effect on SNCA mRNA expression.

Cells transfected with 836R sgRNA plasmid were cell sorted to determine the relative expression of SNCA mRNA in mixed versus pure or homogenous population of cells (FIG. 25).

### Example 7

### Optimization of sgRNA SNCA Promoter Candidates that Downregulate SNCA Gene Expression in Human iPSC-Derived Neural Stem Cells

Newly designed sgRNAs (for example, as shown in Table 3) are tested against the SNCA promoter in cell and human stem cell models expressing dCas9-KRAB. HEK293 line with doxycycline-inducible CRISPR/dCas9-KRAB co-expressing GFP and a human iPSC line with doxycycline-inducible CRISPR/dCas9-KRAB co-expressing mCherry is used.

Specificity and potential off-target effects of the constructs are addressed by using RNA profiling (RNA-Seq) and BLESS technology (direct in situ breaks labeling, enrichment on streptavidin and next-generation sequencing) to capture Cas9-induced DNA double-stranded breaks (DSBs) in human iPSC-derived neurons. RNA Seq and BLESS is carried out on differentiated neurons transduced with viral constructs containing dCas9/sgRNA and corresponding controls: 1) CRISPR/dCas9 with sgRNA not targeting the SNCA promoter, 2) CRISPR/dCas9 without a targeting sgRNA. Cells are analyzed in triplicates after 35 days in vitro.

The documented and published sgRNAs and newly designed sgRNAs are tested in HEK293 cells. Cells are treated with doxycycline to induce dCas9-KRAB expression followed by transduction of the sgRNA constructs. SNCA mRNA expression using Taqman technology is performed 48 hrs after transfection. Of the 20 tested sgRNAs, depending on the robustness of knockdown, up to 6 sgRNAs are selected to test in human iPSC-derived neuronal cultures expressing dCas9-KRAB together with mCherry. Successful candidates used for further testing reach a >/=50% reduction in SNCA gene expression.

In parallel to testing and optimizing sgRNAs as described above, other CRISPR/dCas9 fusion proteins, for e.g. DNMT3A to induce DNA methylation which would permanently change SNCA gene expression, is also tested and optimized as an alternative approach. Other identified regulatory regions in the SNCA gene are also explored via CRISPRi.

### Example 8

### Identify Level of Knockdown of Alpha-Synuclein Needed of the dCas9/sgRNA system to Assess Functional Recovery In Vitro by Transduction in Patient Derived iPSC-Derived Neurons with SNCA Multiplication

Alpha-Synuclein is Robustly Expressed in iPSC-Derive Neuronal Cultures.

Human iPSC lines from a patient with an SNCA genomic triplication was generated, resulting in a 2-fold increase of alpha-synuclein protein expression compared to the sibling control. iPSC-derived progenitors and differentiated neurons are used from this patient who had an early onset and a very rapid disease progression with dementia. In iPSC-derived neuroprogenitors from this patient, cellular phenotypes were rescued by RNA interference knockdown of alpha-synuclein. alpha-synuclein mRNA and protein levels in human iPSC-derived neural stem cells and differentiated neurons is detected. This well-characterized and validated human stem cell model is used as a tool to test downregulation of alpha-synuclein mRNA and protein. As a control for gene dosage expression of SNCA, the isogenic iPSC lines carrying different gene copy numbers of the functional SNCA gene using CRISPR technology (generated in Example 2). Frameshift mutations in SNCA genomic triplication cell line are sequentially induced, generating different endogenous levels of alpha-synuclein protein (2-fold, 1.5-fold, 1-fold, 0.5 fold, and no protein expression). These cells are used in comparison with the sgRNAs transfected neurons to assess changes in the functional assays and to titrate downregulation as described below.

High-Efficiency Neuronal Differentiation into Dopaminergic Neurons.

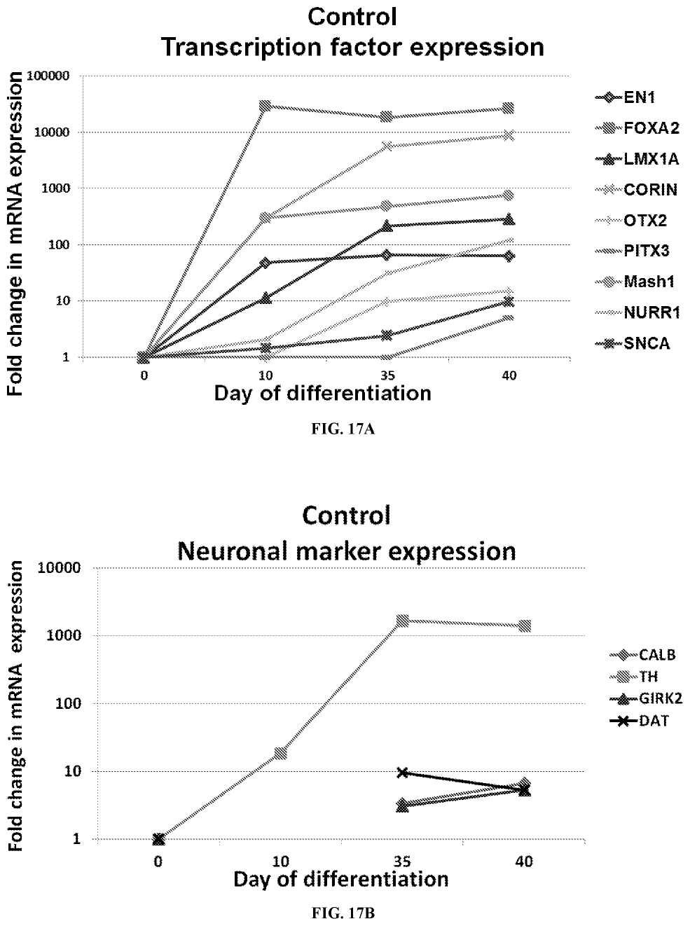

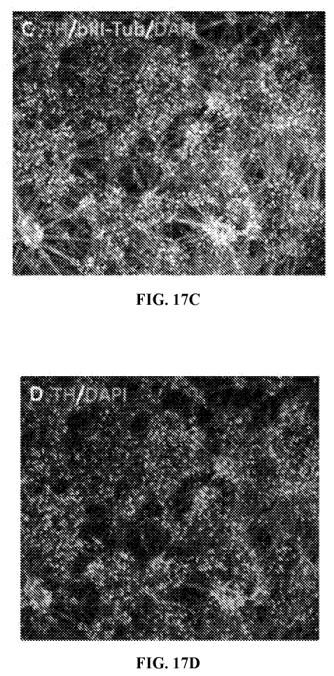

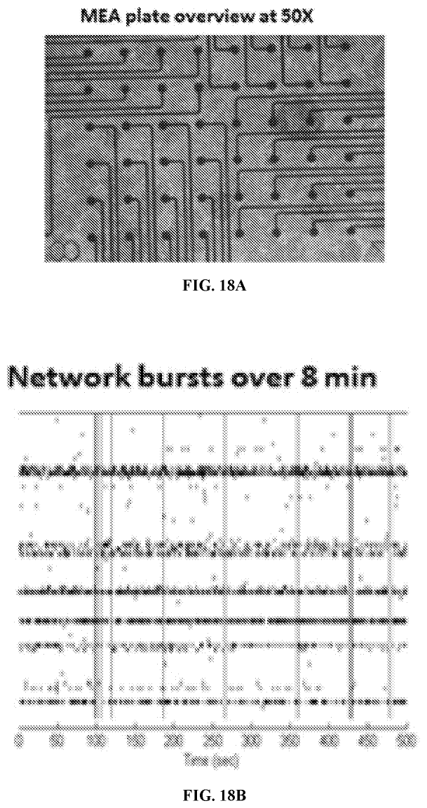

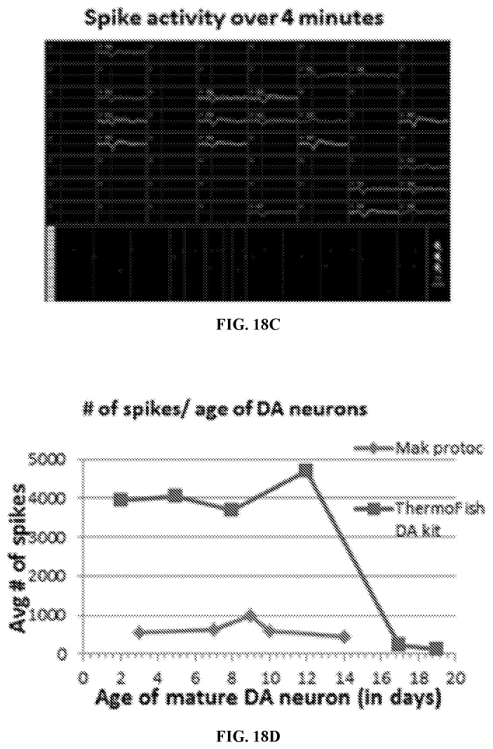

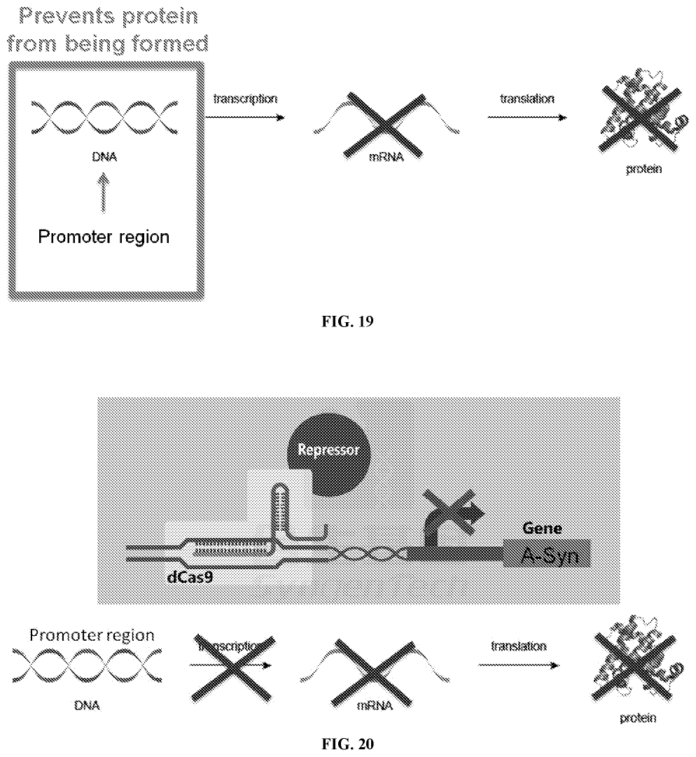

A robust and reproducible protocol is used to differentiate patient-derived iPSCs into neuronal cultures within 35 days with high-efficiency of ˜60% dopaminergic neurons of the total cell population (DAPI cell counts). The culture is induced to derive floorplate progenitors (high FOXA2 expression, FIG. 17A). In the maturation phase, which takes 15-20 days, the cells mature into dopaminergic neurons which show spontaneous synaptic activity measured in multielectrode arrays (FIG. 18A-FIG. 18D).

Determine Level of CRISPR/dCas9-KRAB Knockdown of Alpha-Synuclein Needed for Functional Recovery In Vitro.

To test constructs that are further used for the in vivo experiments in Example 7, a lentivirus construct containing the CRISPR/dCas9-KRAB and their corresponding sgRNAs (optimized in Example 6 and 7) is built and differentiated human iPSC-derived neuronal cultures are directly infected.

Alpha-synuclein mRNA is measured by Taqman expression assay and protein by immunoblot, immunocytochemistry for tyrosine hydroxylase, beta-III-tubulin, and alpha-synuclein is carried out on 8-well plates. Further, cytotoxicity (Sytox and Cas3/7), cellular stress (MitoSox/ROS, and mitochondrial membrane potential JC-10) is measured. All assays are established and design and statistics have been previously described. The synaptic activity on MEA arrays is also analyzed to assess any changes in spike activity or pattern as exemplified in FIG. 18A-FIG. 18D.

A knockdown of alpha-synuclein at the mRNA and protein level in differentiated neurons is detected. The iPSC CRISPR edited SNCA isogenic controls help with the assessment of the phenotypes. The knockdown of alpha-synuclein in a cell line with an SNCA gene triplication, results in a rescue of cellular phenotypes. sgRNA constructs that exhibit a=/>50% reduction in alpha-synuclein expression at the mRNA and protein level are tested in the in vivo SNCA PAC transgenic model (Example 8).

### Example 9

### Identify AAV CRISPR/dCAS9 Regiment in the In Vivo SNCA PAC Transgenic Mouse Model that Provides the Level of Knockdown that Rescued Human iPSC Neurons

For the purpose of in vivo proof-of-principle of the alpha-synuclein CRISPR interference approach, a transgenic mouse model is utilized. This model has a P1 artificial chromosome (PAC) encoding the entire human SNCA gene (including promoter and regulatory elements) and mice homozygous for the wild-type human SNCA (PAC-Tg (SNCA)^{+/+}) bred onto the SNCA^{-/-} background are generated. These mice are crossbred to a mouse strain that carries a dopamine transporter promoter-driven cre-recombinase gene which selectively expresses cre recombinase in dopaminergic. This allows for selective assessment of alpha-synuclein levels in dopaminergic neurons and compare them to the in vitro studies in human iPSC-derived dopaminergic neurons (Example 8) and titrate the level of downregulation.

More specifically, a Cre/Lox-switch for the CRISPR/dCas9 sgRNA system is built. This only expresses CRISPR/dCas9 in neurons that also express Cre recombinase which is in turn controlled by the DAT promoter in dopaminergic neurons. For these experiments, the three strongest CRISPR/dCas9 sgRNAs targeted to the human SNCA promoter (=/>50% reduction in SNCA gene expression in human iPSC-derived dopaminergic neurons) that emerged from the screen in human iPSC-derived neural stem cells and neurons are tested along with the known sgRNAs that reach a 50% knockdown in human iPSC-derived neurons. As control conditions, three different vectors are used: 1) CRISPR/dCas9-KRAB with a scrambled sgRNA (not targeting a sequence in the human genome), 2) CRISPR/dCas9-KRAB without a targeting sgRNA, and 3) empty AAV6 vector. 10 animals per experimental and control group at 3 months of age are used. Animals are euthanized 4 weeks after surgery and the tissues are collected and processed as described below. The experiments are replicated and alpha-synuclein mRNA and protein expression in the substantia nigra is assessed.

Results of this example, among other things, provide a proof-of-concept for CRISPR/dCas9 interference of the SNCA promoter region delivered by gene therapy and compares data in vivo with the level of knockdown of alpha-synuclein that rescued human iPSC neurons. These experiments lay the foundation for subsequent preclinical studies in rodents, and for studies in non-human primates.

The SNCA PAC mouse line is available from Jackson Laboratories (https://wwwjax.org/strain/010710) and the animals are obtained as breeding pairs to establish a colony. A breeding colony is established with 5 males that are mated with 10 females in each round and resulting off-springs constitute a group that is used in a balanced allocation between each experimental group. This breeding cycle is repeated a number of times during the course of the experiments to obtain the sufficient number of animals for complete analysis.

For generation of AAV viral preparations, the constructs that yield successful and specific suppression of SNCA promoter activity are cloned into appropriate transfer plasmids. Recombinant adeno-associated viral vectors serotype 6 (rAAV6) containing a synapsin 1 promoter (syn1) expressing appropriate CRISPR/dCas9 constructs from a flex-switch cassette flanked with two lox sites on each end in opposing direction followed by a WPRE and then an h-SV40 polyA sequence. These cassettes are flanked by inverted terminal repeats from AAV serotype 2 and preparation of the virus has been previously described.

In order to ensure best targeting and correct delivery in the brain, techniques and procedures in the have been established. In brief, viral vector injections are performed under 1.5% isoflurane anesthesia using O₂ and N₂O gas mixture. The injections are made into the right substantia nigra using a 5 μL Hamilton syringe fitted with a pulled glass capillary which has an outer diameter of 60-80 μm at the tip and a long tapering length. 1.5 microliter of viral preparation is injected at a speed of 0.1 μL/15 sec. The needle is left in place for an additional 5 min before it is withdrawn slowly out of the brain parenchyma. Coordinates used for SN injections are anteroposterior (AP): −2.4 mm and mediolateral (ML): −1.4 mm relative to the bregma and dorsoventral (DV): −4.3 mm from the dural surface, calculated according to the mouse atlas of Franklin and Paxinos (Franklin and Paxinos, 2008). Brains to be processed for histological processing are post-fixed in 4% PFA solution for 2 h before being transferred into 25% sucrose solution for cryoprotection, where they are kept until they had sink (typically 24-36 hrs). The brains are then sectioned in the coronal plane on a freezing microtome at a thickness of 30 μm, sections collected in 6 series and stored at −20° C. until further processing. Immunohistochemical staining is performed on free-floating sections. The primary antibodies used for immunohistochemical staining for detection of alpha-synuclein protein is a mouse anti-a-syn (4B12, Human, does not cross react with mouse, Covance), or rabbit anti-Pser129 α-syn (ab51253, Abcam). The sections are treated with avidin-biotin-peroxidase complex (ABC Elite kit, Vector Laboratories) and the color reaction developed by incubation in 0.5 mg/mL 3,3′-diaminobenzidine and 0.01% H₂O₂. To detect differences in transcribed mRNA, a mRNA in situ hybridization strategy RNAScope is employed which is a relatively novel in situ hybridization technology that allows for signal amplification which suppresses background and preserves morphology. Formalin-fixed, paraffin-embedded midbrain sections are studied from both hemispheres.

Statistical comparisons between groups are conducted with the SPSS statistical package (SPSS Inc., Chicago, Ill.). The data is analyzed by appropriate analysis of variance. For example, the data is sampled from a normal distribution and parametric statistics are appropriately used. When appropriate sources of variance are significant, post hoc tests are undertaken using Tukey's HSD when Levene's test was significant or Dunnett's T3 test is used.

In case of a tradeoff between maximal targeting to achieve efficiency and spread of virus to unplanned brain regions that may result in unanticipated side effects, published data both in larger animal species as well as small clinical trials is used to address the virus spread. Most importantly, the expression level of the constructs in the brain is controlled and adapted, and its duration is defined and terminated as and when needed. Furthermore, in the case of Parkinson's disease, even though dopaminergic neurons in the substantia nigra are the most vulnerable cell population, neurons in adjacent areas of the brain are also affected by alpha-synuclein toxicity. This reduces the risk of negative consequences in case of reducing alpha-synuclein in other cell types to which the virus could potentially spread and it may actually be therapeutically beneficial.

### Example 10

### Advancement of Therapeutic Candidate into Next Stages of Clinical Translation

A target product profile for the therapeutic candidate is developed with i.e. indications and usage, dosage and administration, clinical pharmacology, mechanism, of action, competitive and IP assessment, regulatory path to use.

### Example 11

### Optimization of sgRNA SNCA Promoter Candidates that Downregulate SNCA Gene Expression in Human iPSC-Derived Neural Stem Cells

Preliminary screening in HEK293T cells transiently transfected revealed 4 sgRNAs that met the criteria of showing≥50% reduction in SNCA mRNA expression. Further, SNCA mRNA downregulation was confirmed by generating stable human patient-derived iPSC and neural stem cell lines with lentivirally integrated Sad/Cad9 and sgRNA.

Derivation of Heterogeneous and Clonal sadCas9 Human iPSCs from SNCA Genomic Triplication Carrier and Healthy Control

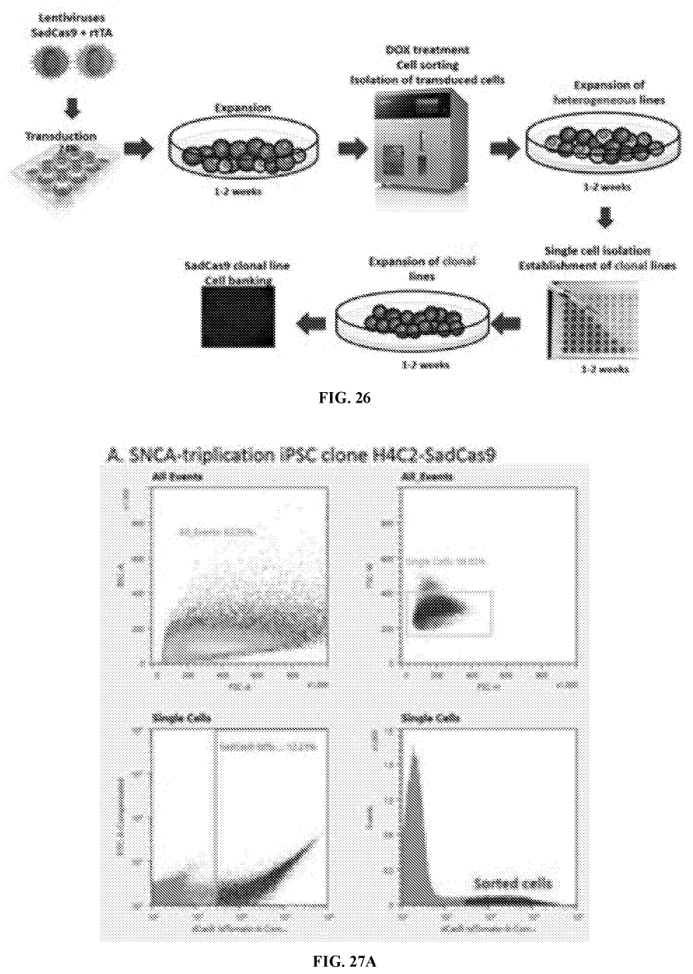

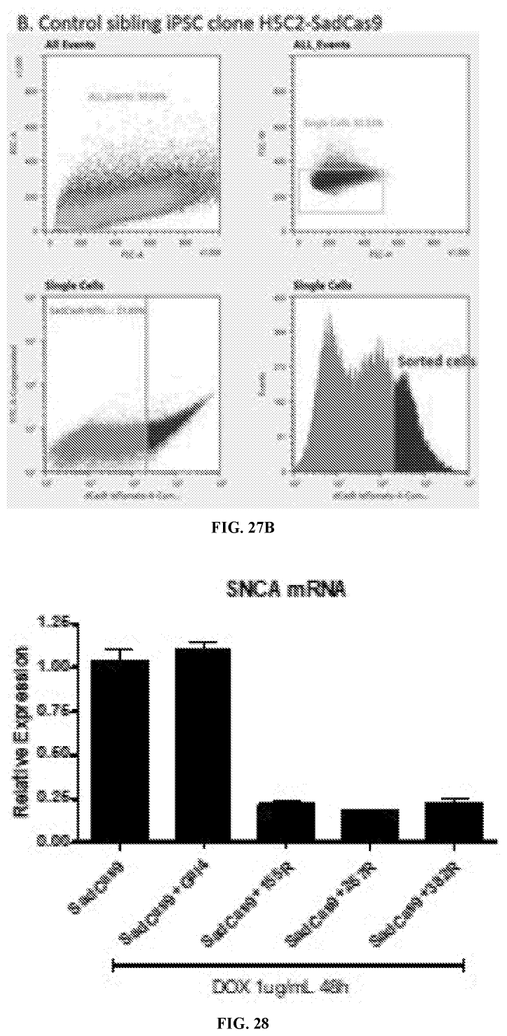

Heterogeneous and clonal human iPSC lines stably expressing SadCas9 were established (FIG. 26). Human iPSCs derived from a patient carrying an SNCA genomic triplication (clones H4C2 and H4C17) and control cell line from an unaffected sibling (clones H5C2 and H5C3) were infected with lentiviruses SadCas9::tdTomato and a reverse tetracycline-controlled transactivator (rtTA). The latter is needed to turn on the inducible promoter controlling the expression of SadCas9. Infected cells were expanded for 1-2. 24 h prior to cell sorting, cells were treated with 1 ug/mL of doxycycline (DOX) to activate expression of SadCas9. Cells were then FACS-sorted for red fluorescence (tdTomato) (FIG. 26 and FIG. 27). Sorted cells were expanded for 1-2 weeks and cryopreserved.

Using this process, SadCas9-iPSCs, SadCas9-HEK293T and SadCas9 SHSY-5Y lines were established by transduction of SadCas9::dtTomato lentivirus and FACS sorting. These sadCas9 sorted cells are composed of a heterogeneous population of cells with different lentiviral insertions for sadCas9. Lentiviral integration at multiple sites, in some instances, results in varying levels of SadCas9 expression and interfere with or confound downstream analyses.

To control the variation of sadCas9 expression levels, single cell isolation/cloning was performed (FIG. 26). Three clones were generated from one original iPSC clones for the SNCA triplication patient (H4C2) and the sibling control (H5C3). A list of clonal human iPSC lines is provided in Table 4. Three additional sadCas9 sub-clones are generated from a second iPSC clone for each the SNCA triplication patient (H4C17) and sibling control (H5C2). The clonal isolation ensures that each resulting human iPSC clone has a uniform expression of SadCas9, therefore differences observed between sgRNAs are not result from varying levels of nuclease expression. Clones were expanded for downstream experiments that express intermediate level of sadCas9 (Table 4). Clones that also concurrently express the selected sgRNAs are being generated to establish clones that will reduce SNCA by 50% and by 75% and assess whether there is a beneficial or adverse effect of downregulation of SNCA gene expression.

sgRNA-Mediated Downregulation in Heterogeneous sadCas9-Expressing iPSCs from Patient with SNCA Genomic Triplication

Prior to the isolation of clonal lines, lentiviral sgRNAs were transduced into heterogeneous SadCas9 cell lines to evaluate downregulation of SNCA. SadCas9-H4C2 iPSCs were infected with sgRNA::BFP lentiviruses. 24 h prior to sorting, cells were treated with 1 ug/mL of DOX to activate expression of SadCas9. Cells were sorted for both red and blue fluorescence, indicating successful integration of SadCas9 and sgRNA. IPSCs were plated into 12-well plates for collection of RNA and evaluation of SNCA mRNA expression. Cells were treated with DOX for 48 hrs and cell pellets were collected. Downregulation of SNCA mRNA is successfully seen by sgRNAs 155R, 267R and 382R (all in TSS2 site) in human iPSC carrying the SNCA genomic triplication (FIG. 28).

sgRNA-Mediated Downregulation in Clonal sadCas9-Expressing HEK293T Cells and iPSCs from Patient with SNCA Genomic Triplication

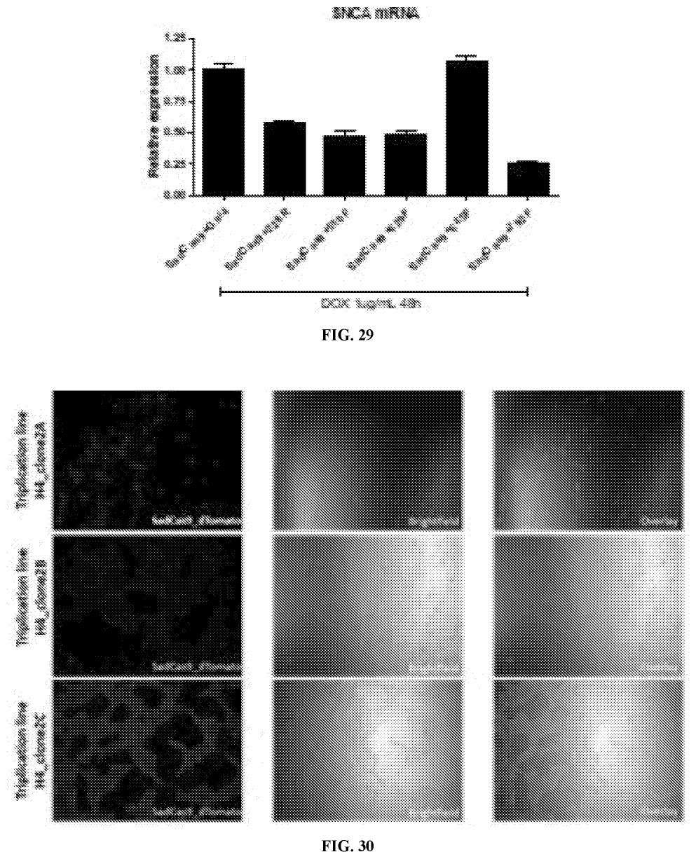

After clonal selection, infection of sgRNAs was performed and SNCA mRNA expression was evaluated. SadCas9-HEK293T clone F3 (HEK-F3) was selected for gene expression analyses. Level of downregulation seen for the clonal line follows the pattern of transient transfections and is comparable (FIG. 29).

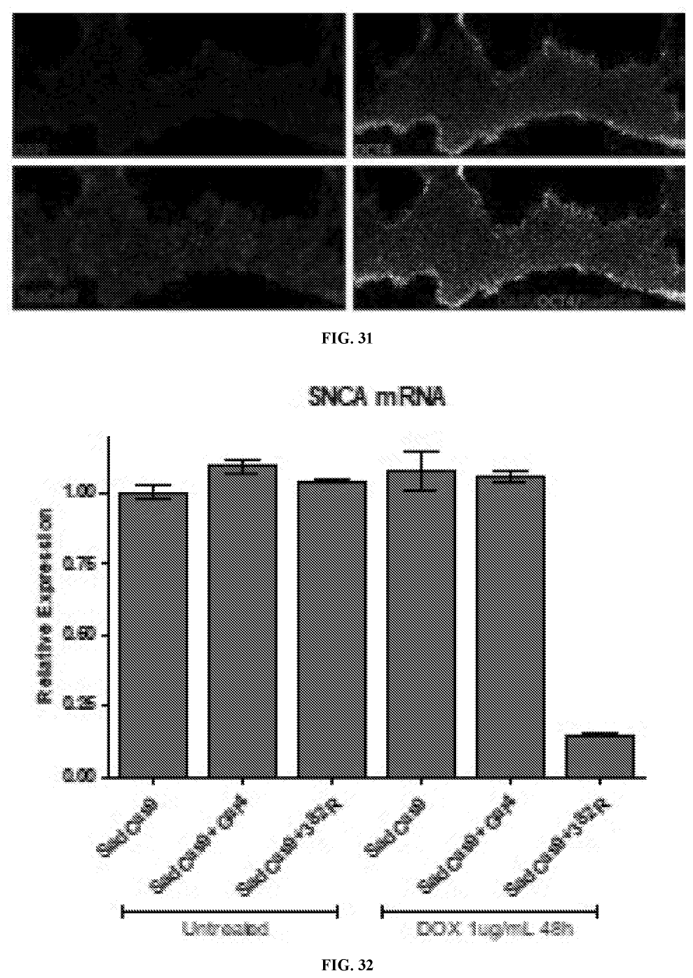

Next, human iPSC sub-clones expressing sadCas9 were isolated. FIG. 30 shows three human iPSC clones (H4C2A, H4C2B, H4C2C) with different levels of SadCas9 expression. Clone H4C2B was chosen for further experiments due to growth and attachment rates being similar to the parental human iPSC line H4C2. Finally, H4C2B iPSC clone was evaluated for expression of OCT4 to ensure they maintain pluripotency after lentiviral integrations (FIG. 31).

Next, SNCA mRNA expression in a clonal iPSC line from the SNCA genomic triplication patient (H4C2) was evaluated with a control sgRNA Gal4 and a sgRNA-382R from TSS2. The CRISPR system capitalizes on a DOX-inducible promoter to control expression of SadCas9, allowing time-controlled activation of the CRISPR interference. When iPSCs were not treated with DOX no change in SNCA mRNA expression was detected, only with addition of DOX SNCA downregulation was detected (FIG. 32). This means that that the system can be activated only in the presence of DOX. A highly significant level of downregulation (>75%) was achieved in human iPSC, confirming suitability of the system for subsequent experiments in Milestone 2 that will evaluate rescue of cellular phenotypes and transcriptional signatures.

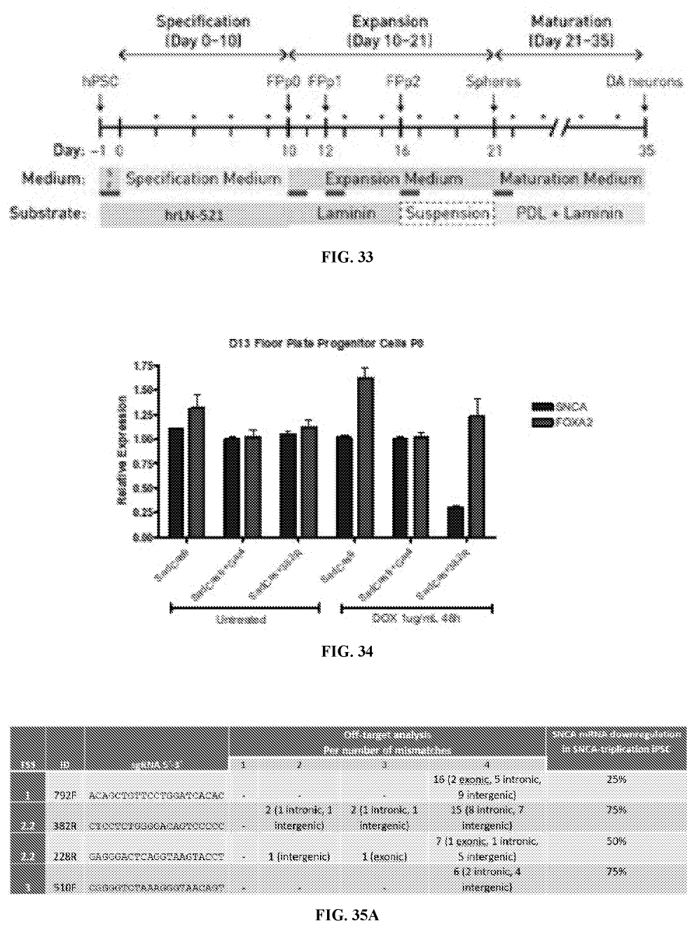

To evaluate SNCA mRNA downregulation in human iPSC-derived neural stem cells, H4C2B, H4C2B-Gal4 and H4C2B-382R were induced to differentiate into dopaminergic neurons in vitro (FIG. 33). A protocol for dopaminergic differentiation which results in 30-35% tyrosine hydroxylase positive neurons derived through intermediate floorplate progenitors has been established.

SNCA Downregulation in Human iPSC-Derived Neural Stem Cells

Floor plate progenitor (FPp) cells were generated after 10 in specification media. At day 10, cells were re-plated for expansion and banking. A portion of these cells were plated into 12-well plates and treated with 1 ug/mL DOX on day 11. FPp cell pellets were then collected at day 13 for RNA extraction.

Evaluation of SNCA mRNA expression in FPp cells followed the same pattern seen in the undifferentiated iPSCs. Robust SNCA mRNA downregulation occurs only when SadCas9 and sgRNA anti-SNCA are activated by treatment with DOX (FIG. 34). SNCA downregulation was ˜75% comparable to the data in undifferentiated human iPSCs.

This demonstrates that the CRISPRi system disclosed herein is able to downregulate SNCA in both iPSC and in neural stem cells derived from a patient with an SNCA genomic triplication.

TABLE 4
Human iPSC lines generated by lentiviral integration of CRISPRi system

| Mutation | Cell line_Clone | Passage | # of vials |
|---|---|---|---|
| SNCA triplication | Huff_C2 (heterogeneous population) | 23 | 5 |
| | | 24 | 6 |
| | | 25 | 5 |
| | | 26 | 4 |
| | Huf4_C2A (Clonal line) | 29 | 6 |
| | Huf4_C2A-Gal4 (SadCas9 + sgRNA) | 32 | 4 |
| | Huf4_C2A-382R (SadCas9 + sgRNA) | 32 | 4 |
| | Huf4_C2B (Clonal line) | 29 | 4 |
| | | 30 | 2 |
| | | 32 | 3 |
| | Huf4_C2B-Gal4 (SadCas9 + sgRNA) | 32 | 2 |
| | Huf4_C2B-382R (SadCas9 + sgRNA) | 32 | 2 |
| | Huf4_C2B-228R (SadCas9 + sgRNA) | 35 | 3 |
| | Huf4_C2B-510F (SadCas9 + sgRNA) | 35 | 3 |
| | Huf4_C2B-629F (SadCas9 + sgRNA) | 35 | 3 |
| | Huf4_C2B-645F (SadCas9 + sgRNA) | 35 | 3 |
| | Huf4_C2B-792F (SadCas9 + sgRNA) | 35 | 3 |
| | Huf4_C2B-564R (SadCas9 + sgRNA) | 39 | 2 |
| | Huf4_C2B-571F (SadCas9 + sgRNA) | 39 | 2 |
| | Huf4_C2B-532R (SadCas9 + sgRNA) | 39 | 2 |
| | Huf4_C2B-317R (SadCas9 + sgRNA) | 39 | 2 |
| | Huf4_C2B-836F (SadCas9 + sgRNA) | 39 | 2 |
| | Huf4_C2C (Clonal line) | 29 | 2 |
| | | 30 | 4 |
| | | 32 | 3 |
| | Huf4_C2C-Gal4 (SadCas9 + sgRNA) | 32 | 4 |
| | Huf4_C2C-382R (SadCas9 + sgRNA) | 32 | 4 |
| | Huf4_C17 (heterogeneous population) | 29 | 3 |
| | Huf5_C3 (heterogeneous population) | 23 | 2 |
| | | 24 | 1 |
| Control Sibling | Huf5_C3A (Clonal line) | 25 | 3 |
| | | 26 | 2 |
| | Huf5_C3C (Clonal line) | 25 | 3 |
| | | 26 | 2 |
| | Huf5_C3D (Clonal line) | 25 | 3 |
| | | 26 | 2 |
| | Huf5_C3C-Gal4 (SadCas9 + sgRNA) | 27 | 4 |
| | Huf5_C3C-202F (SadCas9 + sgRNA) | 27 | 4 |
| | Huf5_C3C-438F (SadCas9 + sgRNA) | 27 | 4 |
| | Huf5_C3C-629F (SadCas9 + sgRNA) | 27 | 4 |
| | Huf5_C3C-645F (SadCas9 + sgRNA) | 27 | 4 |
| | Huf5_C3C-792F (SadCas9 + sgRNA) | 27 | 4 |
| | Huf5_C3C-228R (SadCas9 + sgRNA) | 31 | 1 |
| | Huf5_C3C-510F (SadCas9 + sgRNA) | 31 | 1 |
| | Huf5_C2 (heterogeneous population) | 25 | In progress |

Successful identification of four sgRNAs is completed and off-target effects of the sgRNAs is being studied.

To ensure specificity and address off-target effects of the CRISPR/SadCas9 guide RNA system, a two-step approach is being used: 1. Focused in-silico homology screen with targeted expression analysis of potential off-targets, 2. Global genome-wide screen using chromatin immunoprecipitation (ChIP)-Sequencing compared to RNA-Sequencing in human iPSC-derived neuronal cultures.

There are several unique aspects to this CRISPR interference technology that cannot be assessed with common targeted or whole-genome sequencing strategies: First, the mutant CRISPR/sadCas9 does have nuclease activity and will not introduce double-strand breaks in the genome, hence high-coverage whole-genome sequencing or Guide-Seq (genome-wide unbiased identification of double-strand breaks enabled by sequencing) for genome-wide profiling of off-target cleavage by CRISPR/Cas9 is not an approach for the detection of off-targets for CRISPR interference. Second, CRISPR/sadCas9 inhibition binding can affect regulation gene expression by sgRNA hybridization to intronic or intergenic regions and its regulated gene target might be located in a distance from sgRNA binding. Third, CRISPR/sadCas9 fused to a KRAB domain (as used herein), can introduce epigenomic changes such as histone modifications which also regulate gene expression by activating or repressing histone marks.

Focused In-Silico Homology Screen with Targeted Expression Analysis of Potential Off-Targets

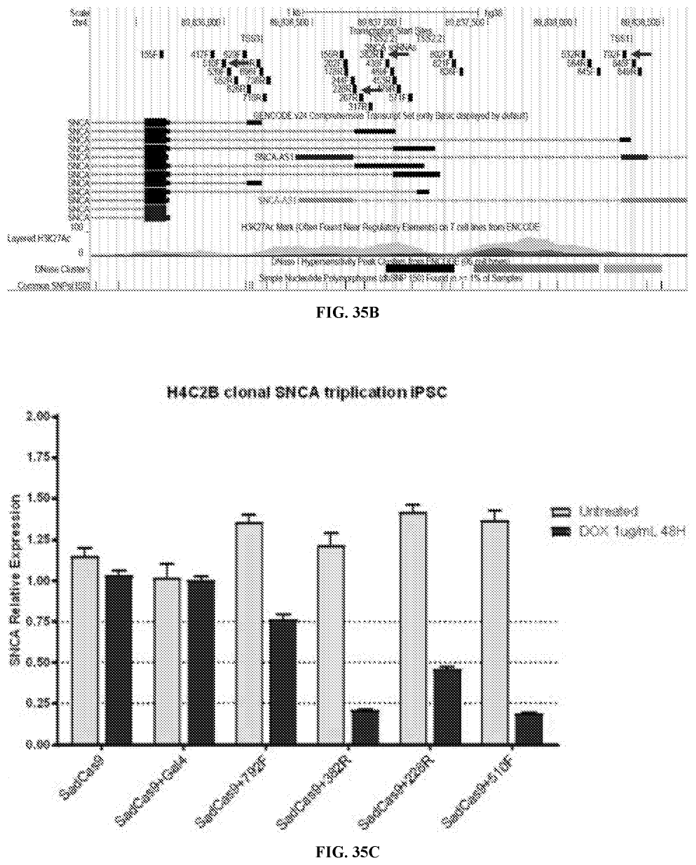

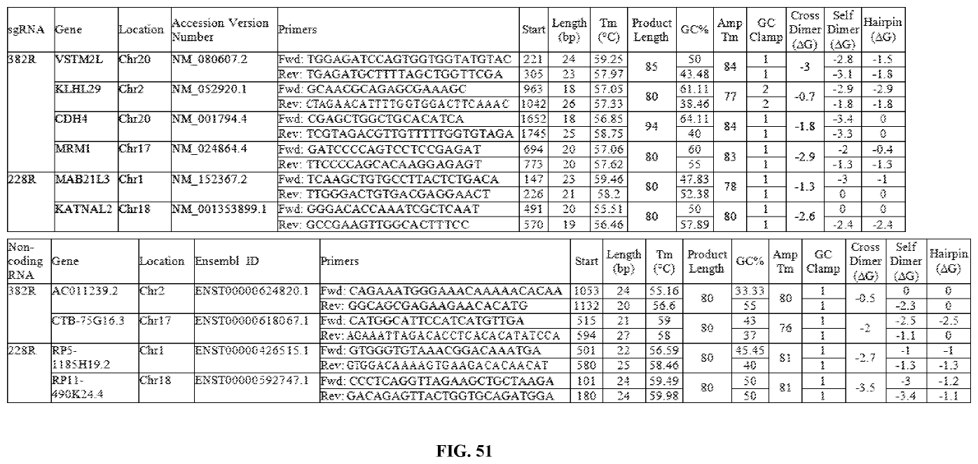

Using CRISPOR bioinformatics off-target analysis (FIG. 35A-FIG. 35C), predicted off-targets for the four lead candidates were analyzed. No mismatches for 1 bp in the 21 nt long sgRNAs were detected. 2 and 3 mismatches for sgRNAs 382R and 228R were detected. For these off-targets, SYBR Green primers were designed for a total of 6 genes and 4 long non-coding RNAs (FIG. 51). Gene expression of these targets is tested. 3 independent neuronal differentiation experiments with 2 independent wells from two clones are performed.

Global Genome-Wide Screen using Chromatin Immunoprecipitation (ChIP)-Sequencing Compared to RNA-Sequencing in Human iPSC-Derived Neuronal Cultures

The second level off-target analysis is an unbiased combined global genome wide screen of ChIP sequencing for sadCas9 (Cas9-DNA interactome) to assess the binding of Cas9 to potential regions that were not predicted by in silico analysis and genome-wide RNA-Seq expression profiling (transcriptome) that allows to compare the sadCas9 binding sites with potential changes in gene expression around these regions. These experiments are carried out in neuronally differentiated human patient-derived iPSCs.

With the ChIP sequencing Cas9 binding sites guided by the four lead sgRNAs are identified, compare peaks to background signal and shape of peak. Peaks to the genome are annotated and calculate distance of peak to the nearest transcription start site (TSS). This allows us to generate a Cas9/lead sgRNA binding profile for the four sgRNAs. This profile is for each sgRNA in neuronally differentiated clones from the SNCA triplication carrier. 3 independent neuronal differentiation experiments were performed with 4 independent wells. Floor plate progenitors (FPp1) were harvested at day 16 of differentiation using ThermoFisher PSC midbrain dopaminergic neuron differentiation kit. Cells were treated w/1 ug/ml of doxycycline for 5 days.

For the computational analysis, a probabilistic method (target identification from profiles or TIP) is used to annotate peaks and rank gene targets and transform all scores into z-scores, assess significance for each gene.

Data for RNA-Sequencing is generated.

Illumina NovaSeq 6000 S2 Reagent Kit is used and mRNA is sequenced using polyA enrichment and sequence in paired end with at least 20 mio clusters. Illumina BaseSpace Suite is used for initial bioinformatic analysis (TopHat Alignment, Cufflinks Assembly, Differential Expression apps). TopHat 2 provides high-confidence alignment for abundance measurement, detection of splice junctions, gene fusions, and cSNPs. Cuffdiff allows transcript discovery and differential expression analysis. The RNA-Seq analysis is performed in collaboration with the Stanford Bioinformatics service core.

For the ChIP/RNA-Seq comparison, first the differentially expressed genes between Cas9/sgRNA and Cas9/negative control Gal4 from RNA-seq are determined and compare target peaks from ChIP-seq for the same experimental samples. BETA target analysis is used to calculate each gene's regulatory potential between ChIP peak and TSS within 100 kb of TSS 3. In addition, both activation or repression of Cas9/sgRNA is determined using the GREAT algorithm even though we assume that Cas9 binding will primarily inhibit gene expression.

Computational analysis of the RNA-Seq data, perform ChIP-Sequencing, and targeted expression analysis with SYBR Green assays is performed.

### Example 12

### Identify Level of Knockdown of Alpha-Synuclein Needed of the dCas9/s₂RNA System to Assess Functional Recovery In Vitro by Transduction in Patient Derived iPSC Derived Neurons with SNCA Multiplication

Different pathways and cellular dysfunction are implicated in the pathogenesis of neurodegeneration in Parkinson's disease. Currently, the most investigated phenotypes and widely accepted cellular mechanisms are modified and misfolded/aggregated alpha-synuclein, increased oxidative stress, and impairment of the lysosomal/autophagy pathways.

Therefore, several assays in these three areas are applied to better assess functional recovery in the in vitro human iPSC-derived neuronal model: 1. Caspase 3/7 for cellular stress (CellEvent™ Caspase-3/7 Green), 2. detection of reactive oxygen species (CellROX™ Green Assay), 3. lipid peroxidation (Lipid Peroxidation BODIPY™ 665/676), 4. autophagy markers (p62 and LC3), and 5. mitochondrial DNA damage (Sanders et al. 2014).

Four sgRNAs with different levels of alpha-synuclein downregulation were selected (FIG. 35A-FIG. 35C). Two sgRNAs show a 75% knockdown (382R and 510F), one sgRNA exhibits a 50% knockdown (228R), and one sgRNA shows a 25% knockdown (792F) of alpha-synuclein expression to assess functional cellular changes.

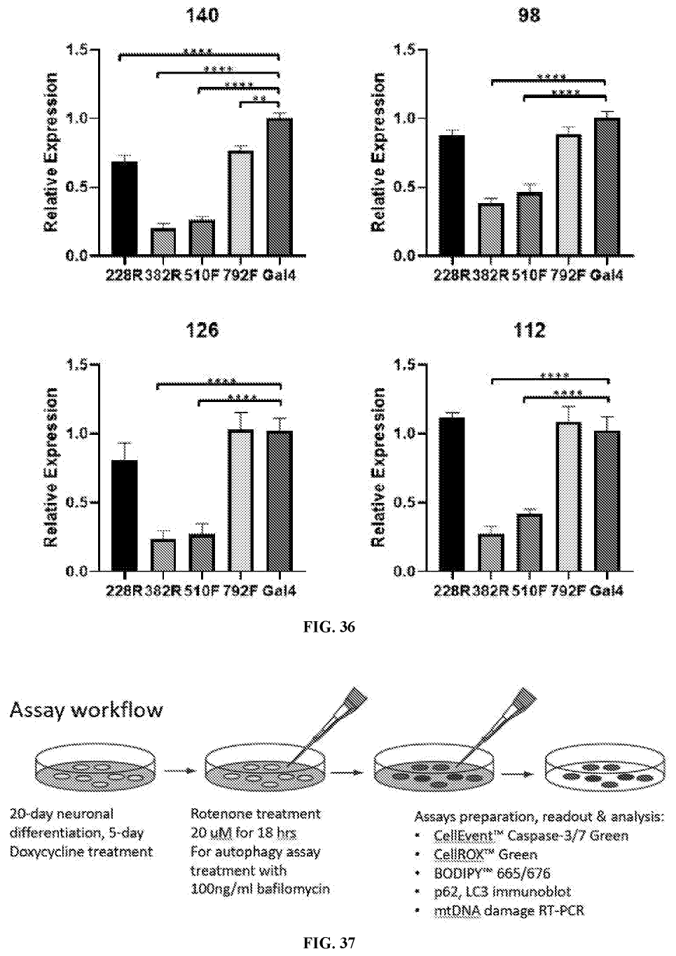

Besides the downregulation of the main full-length form of alpha-synuclein (SNCA-140, 140 amino acids or aa), three shorter isoforms that undergo alternative splicing for exons 3 and 5 (SNCA-126, SNCA-112, and SNCA-98) have been described. Although not much is known about alpha-synuclein gene isoforms and their role in the neurodegenerative process of Parkinson's disease, we wanted to understand if expression of the shorter isoforms is affected by our CRISPR inhibition approach (FIG. 36). Full-length SNCA-140 showed a decreased expression in all the sgRNA lines compared to the control Gal4. Interestingly, while the two sgRNAs with the strongest downregulation (382R and 510F) for the SNCA-140 isoform also affected to a similar extent the three shorter isoforms, the other two sgRNAs did not have an effect on SNCA-126, SNCA-112, or SNCA-98.

Three image-based assays were performed to detect cell stress (CellEvent™ Caspase-3/7 Green), reactive oxygen species and lipid peroxidation (CellROX™ Green Assay) and (Lipid Peroxidation BODIPY™). The workflow is shown in FIG. 37.

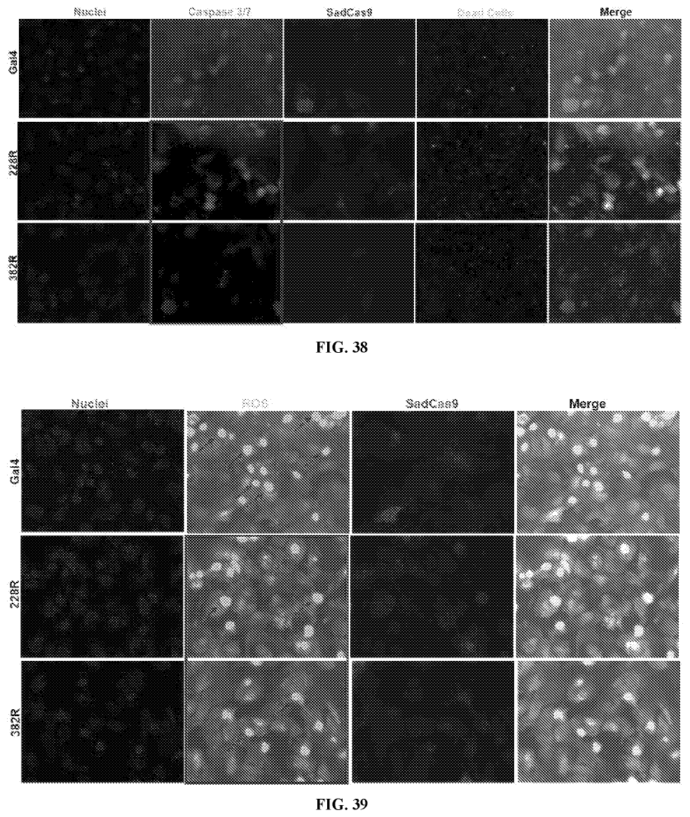

Apoptotic cells with activated caspase-3/7 show bright green nuclei, while cells without activated caspase 3/7 exhibit minimal fluorescence signal fluorescent signal from CellEvent™ Caspase-3/7. A decrease in caspase 3/7 activation is seen for two sgRNAs, one supports a 50% SNCA mRNA downregulation (sgRNA 228R) and the other one (sgRNA 382R) mediates a 75% SNCA mRNA downregulation. The effect seems to be more pronounced for the sgRNA that shows the stronger downregulation (FIG. 38).

The CellROX® oxidative stress reagent is a fluorogenic probe which measures reactive oxygen species (ROS) in live cell cultures. The cell-permeable reagent is weak fluorescent while in a reduced state and upon oxidation exhibits a strong fluorogenic signal. CellROX® Green Reagent is a DNA dye, and upon oxidation, it binds to DNA and its signal is therefore localized primarily to the nucleus (FIG. 39). A decrease in ROS is detected for two sgRNAs, one supports a 50% SNCA mRNA downregulation (sgRNA 228R) and the other one (sgRNA 382R) mediates a 75% SNCA mRNA downregulation. The effect seems to be more pronounced for the sgRNA that shows the stronger downregulation. Fluorescent signal for ROS is ˜60% of the Gal4 signal in sgRNA 228R and ˜45% of the Gal4 signal in sgRNA 382R. Also for the ROS assay, the higher downregulation shows lower ROS (FIG. 39).

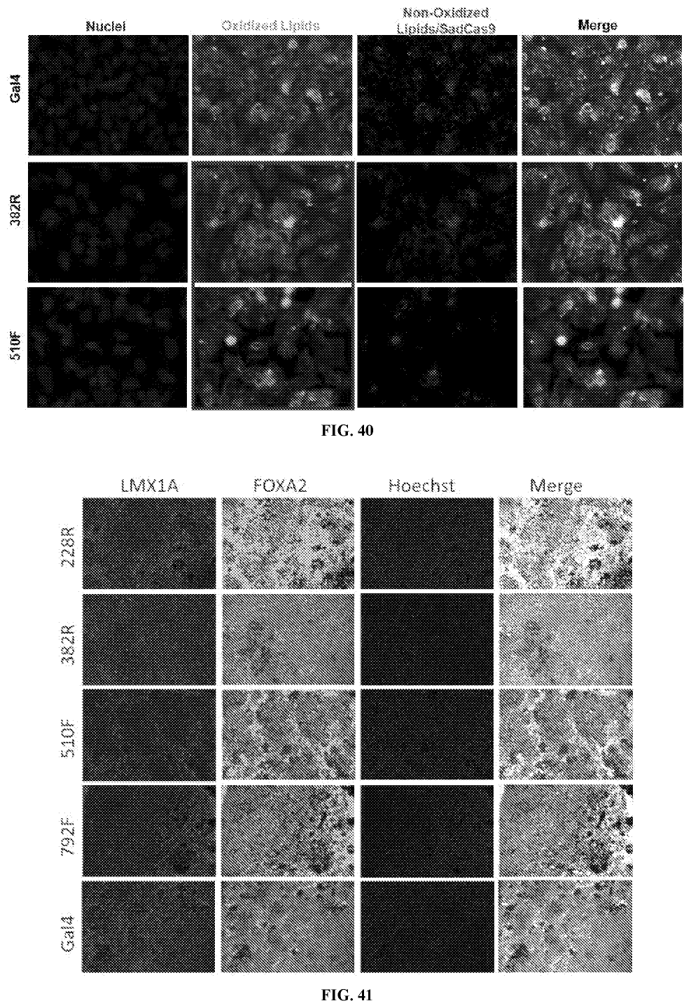

Lipid peroxidation generally refers to the oxidative degradation of cellular lipids by reactive oxygen species. Peroxidation of unsaturated lipids affects cell membrane properties, signal transduction pathways and has been implicated in the pathogenesis of PD with an increase basal lipid peroxidation in the substantia nigra. Lipid peroxidation can be detected with the lipophilic probe, BODIPY® 665/676 dye. This probe exhibits a change in fluorescence after interaction with peroxy1 radicals and the ratio of fluorescence for oxidized lipids to non-oxidized lipids are measured. FIG. 40 shows qualitative images of the assay.

Methods:

Cell culture and floor plate progenitor differentiation—The iPSCs were from a patient with the SNCA triplication (Iowa Kindred). The iPSCs were CRISPR edited with guides at various transcriptional start sites (H4C2B). The edited lines had various amounts of expression of alpha-synuclein depending on their sequences, binding sites and DOX treatment. These iPSCs were cultured under feeder-free conditions in StemFlex™ medium (ThermoFisher) as colonies on Geltrex™ (ThermoFisher, CAT: A1413302, diluted 1:70) for maintenance and single-cell on Laminin LN521 (ThermoFisher, CAT: A29248 [10 μg/μl]) coated plates for DOX treatment and downstream analysis. The maintenance plates were maintained as colonies in 12-well plates, 1 well per clone, and were passaged manually once a week. Manual passaging consisted of scoring the colonies with a 25-gauge needle and then pipetting the scored pieces onto a new plate coated with Geltrex™ and containing StemFlex™ medium and THZ (thiazovivin) (STEMCELL Technologies [1 μg/mL]). THZ was washed off the wells 24 hours after plating the colonies. For DOX treatment, the cells from the maintenance plate were single-cells and plated onto a Laminin coated 12-well plate, 2-wells per clone, seeded at ˜300,000K per well with THZ (washed off 24 hours later) and grown until ˜90% confluent. Once confluent, cells were treated with DOX [1 μg/mL] for 48 hours and then collected. Cells were either manually passaged or passaged via Accutase (ThermoFisher) according to the manufacturer's recommendations.

The iPSCs were differentiated via the PSC Dopaminergic Neuron Differentiation Kit (Thermofisher, CAT: A30416SA) from day 0-10 as floor plate progenitors (FPp1). During the differentiation process, the change that was made to the protocol was that cells were grown as monolayer and not a spheres. At day 14, floor plate progenitors were treated with DOX [1 μg/mL] for 5 days and then collected for all functional assays and expression analysis.

SYBR™ green primer design and gel electrophoresis. SYBR™ green primers were a redesign of the TaqMan® primers previously used. Each primer was designed against the TaqMan® primers and two previously used primers (Bungeroth et al., McLean et al.) to amplify the exon-exon junctions of the various isoforms. The GC content and secondary structure analysis of each primer set was analyzed through Beacon Designer™ Free Edition by Premier Biosoft (http://free.premierbiosoft.com). The parameters in the program that make a good primer are the AG's had to be more positive than −3.5 kcal/mol, the primer pair should not have an annealing temperature greater than 60° C. and that the hairpins avoided involvement of the 3′ end. All primer sets were run together with a Gal4 sample and gel (2% agarose gel) electrophoresis was performed after each qPCR. The master mix used was PowerUp™ SYBR™ Green Master Mix (Thermofisher, CAT: A25742). Primer pairs that showed good Cts and had bands in the gel with similar melting temperatures were chosen to be used on the remaining sgRNA samples.

RNA extraction, cDNA synthesis, and qPCR. RNA extraction was performed using the PureLink™ RNA Mini Kit (ThermoFisher, REF: 12183025) following manufacturer's guidelines. RNA concentration was determined after isolation using the NanoDrop Technologies ND-1000 Spectrophotometer (ThermoFisher). cDNA synthesis was completed using the High Capacity cDNA Reverse Transcription Kit (ThermoFisher, REF: 4368814) following manufacturer's guidelines. cDNA was diluted with nuclease-free water to make a single 100 μL aliquot with 10 ng/μL. For qPCR, the TaqMan® Gene Expression Master Mix (ThermoFisher, REF: 4369016) was used. Relative mRNA expression was calculated by the 2-ΔΔCt method; ΔCt=Target Ct−Reference mean Ct, ΔΔCt=ΔCt sample −ΔCt calibrator. The reference mean was human GAPDH, a housekeeping gene. The calibrator was the RNA sample Gal4 which was used across all runs for normalization.

Caspase 3/7 endpoint assay: 5 μM of CellEvent™ Caspase-3/7 Green Detection diluted in PBS was added with 5% FBS directly into the culture media and the cells were also treated with LIVE/DEAD™ Fixable Far Red Dead Cell Stain Kit (633 or 635 nm excitation) which penetrates cells efficiently and provides a separation of live and necrotic cells. The cells were incubated at 37° C. for minutes, then fixed cultures with 3.7% formaldehyde for 15 minutes and used Hoechst as a nuclear counterstain. The cells were imaged using instrument filter sets for FITC and Alexa Fluor™ 488 dye. The excitation/emission maxima for the CellEvent™ Caspase-3/7 Green Detection Reagent is 502/530 nm. Microscopy was performed in KEYENCE BZ-X700 microscope and the images are being analyzed with BZ-X700 software.

CellROX® Oxidative Stress Reagent assay: The cells were treated with 20 micromolar rotenone for 18 hrs. Then, CellROX® Reagent was added at a final concentration of 5 μM to the cells and incubated for 30 minutes at 37° C. After removal of the medium and the cells were washed three times with PBS and fixed the neuronal cultures with 3.7% formaldehyde for 15 minutes, counterstained with Hoechst and analyzed the signal within 24 hours with KEYENCE BZ-X700 microscope. The images are being analyzed with BZ-X700 software.

Lipid peroxidation assay: Briefly, BODIPY® Lipid Peroxidation Sensor was added at a final concentration of 10 μM to the cells and incubated for 30 minutes at 37° C. After removal of the media, cells were washed three times with PBS. Read the fluorescence at to separate wavelengths; one at excitation/emission of 581/591 nm for the reduced dye, and the other at excitation/emission of 488/510 nm for the oxidized dye. The ratio of the emission fluorescence intensities at 590 nm to 510 nm gives the read-out for lipid peroxidation in cells. Lipid peroxidation is determined by quantitating the fluorescence intensities with KEYENCE BZ-X700 microscope and calculating the ratio of intensity in the red channel to the intensity in the green channel

All raw image data for caspase, ROS, and lipid peroxidation are analyzed. Further immunoblotting for autophagy markers is performed and the mtDNA damage data is analyzed.

### Example 13

### Determine AAV CRISPR/dCAS9 Regimen In Vivo SNCA PAC Transgenic Mouse Model Resulting in an Efficacious Level/Fold Synuclein Knockdown Equal to Rescued Human iPSC Neurons

AAV9 vectors that include the three most efficiently downregulating small guide RNAs (sgRNAs) that show up to 75% downregulation in human iPSC cultures were constructed. The lead sgRNA candidates were based on the off-target profile. Three sgRNAs with the lowest predicted off-target profile were selected (Table 5). The three sgRNAs do not show any off-target prediction with one, two, or three mismatches, only 4 mismatches were detected. Further characterization of on-target and off-target effects are done for future clinical development.

TABLE 5

| | | | In silico |
|---|---|---|---|
| | | | off-target |
| Lead sgRNA candidates | | | predictions |
| 382R | 382rev | CTCCTCTGGGGACAGTCCCCC | 9 intronic with |
| | | | 4 mismatches |
| 267R | 267rev | AAGAGAGAGGCGGGGAGGAGT | 2 exonic, |
| | | | 96 intronic with |
| | | | 4 mismatches |
| 155R | 155rev | GAATGGTCGTGGGCACCGGGA | one intronic with |
| | | | 4 mismatches |

In preparation for the in vivo experiments, we tested two different AAV constructs (AAV9 and AAV9 PHP.B) and different routes of administration (cisterna magna, lateral ventricle, and intraparenchymal (striatum).

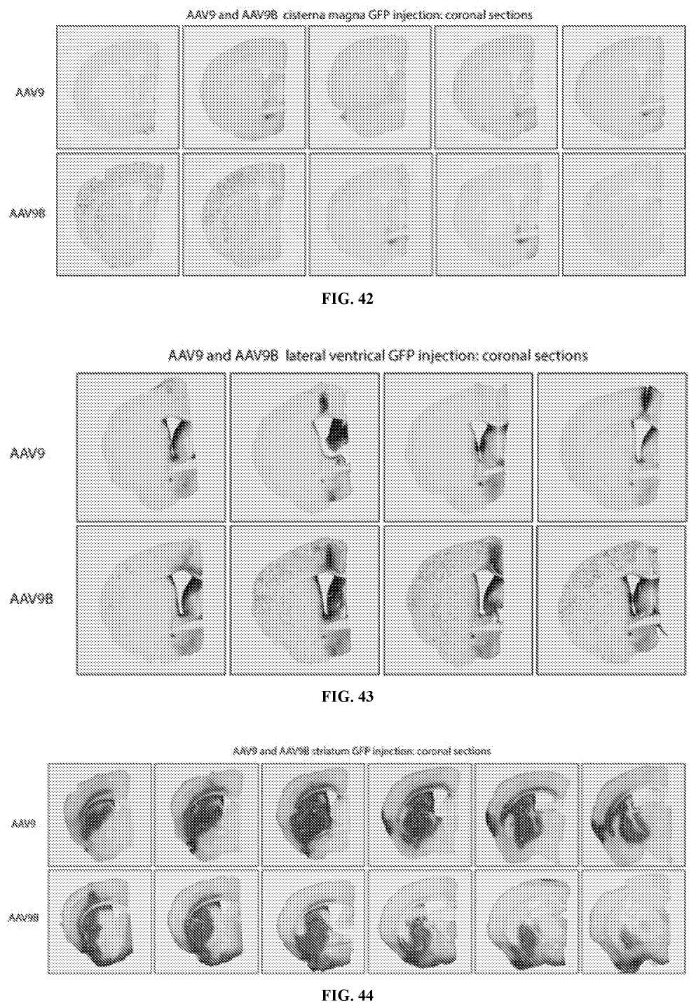

Established AAV9 was compared against a newly published AAV9 PHP.B (Chan et al. 2017, Nat Neuroscience3) to compare transduction when delivered intraventricular versus intraparenchymal. The data show that there is higher transduction of cortical neurons compared to the standard AAV9 construct for cisterna magna (FIG. 42) and lateral ventricle (FIG. 43) delivery. Both AAV constructs showed similar transduction when injected in the striatum (FIG. 44).

For the planned in vivo experiments, we continue with our standard AAV9 vector and intrastriatal delivery. The new capsid variants are tested and evaluated as well.

AAV Design.

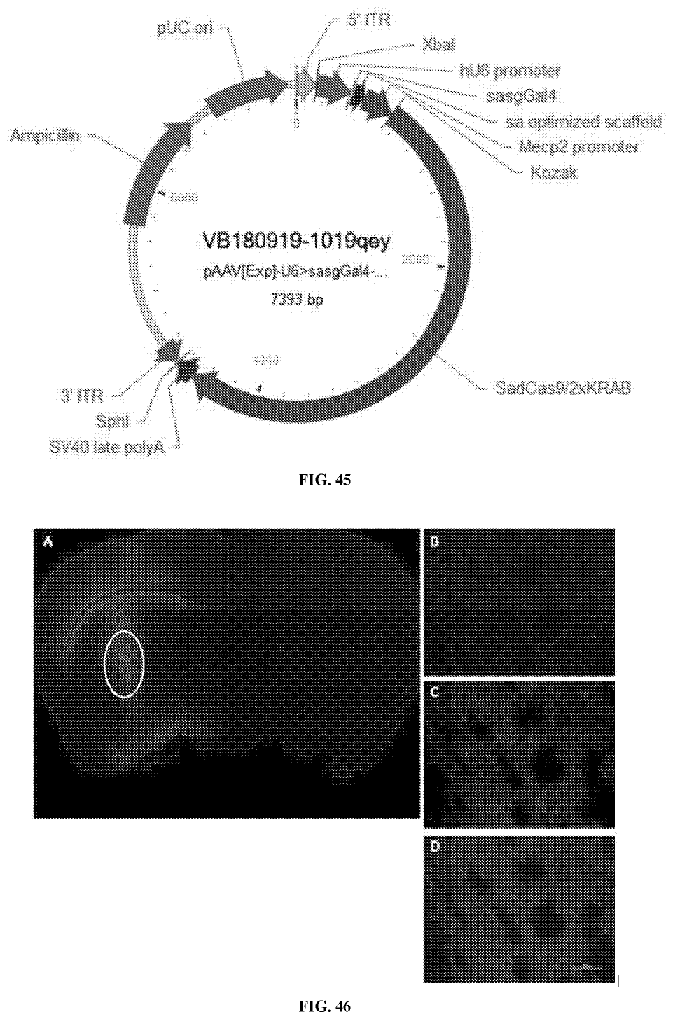

The design of the AAV expression cassette was optimized to fit the CRISPRi/Cas9 2xKRAB construct together with a selected lead candidate guide RNA in one construct. The human MECP2 promoter was used and include a SV40 late polyA termination signal for sadCas9 expression. The lead sgRNA is co-expressed from the same vector which is implemented by using the human U6 promoter (75 bp shorter than the mouse U6 promoter). With this design, the packaging capacity of 4.8 kb is reached including ITR domains of the AAV (FIG. 45). Serotype for the AAV, an AAV9, namely AAV-PHP.eb, which has been shown to achieve higher transduction in the CNS based on recent developments, e.g. clinical trial for SMA1, is used. In vivo rodent studies showed that, compared to PHP.B and AAV9, intravenous injection of PHP.eB AAV led to an increase in both the number of transduced cells and the expression level per cell. In vivo, PHP.eB transduced the majority of neurons in the cortex and striatum, and over 75% of cerebellar Purkinje cells. Preliminary studies to test the systemic spread of the virus were also conducted.

The mouse colony has been established through homozygosity mating and mice for this study were born within 3 weeks. The mice carry four mutant copies of the human alpha-synuclein locus comparable to the human condition of the SNCA genomic triplication.

Optimization of stereotactic injections: To optimize the surgeries, stereotaxic dye injections were performed in three adult mice followed by histological examination to determine the site of injection and the extent to which the injectate spreads within the brain tissue. With these optimization steps, the exact coordinates for the striatum (AP +0.5, ML −2.3, DV −3.2) and substantia nigra (AP −3.2, ML −1.4, DV −4.3) in adult male and female animals were determined.

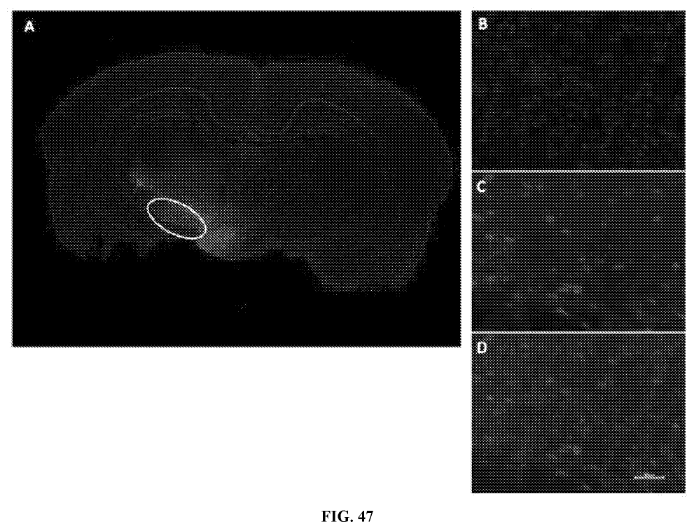

Spread and expression of AAV-PHP.eb virus is shown by tdTomato fluorescence in the striatum (FIG. 46) and the substantia nigra (FIG. 47). The transduction efficiency of the AAV-PHP.eb is comparable with other serotypes such as AAV5 and AAV9. With directed stereotaxic delivery of the virus in the target brain regions, we are able to assess the effect of the CRISPR/sadCas9 system and optimize our lead sgRNA candidates.

Optimization of RNA Hybridization Technology (RNAScope).

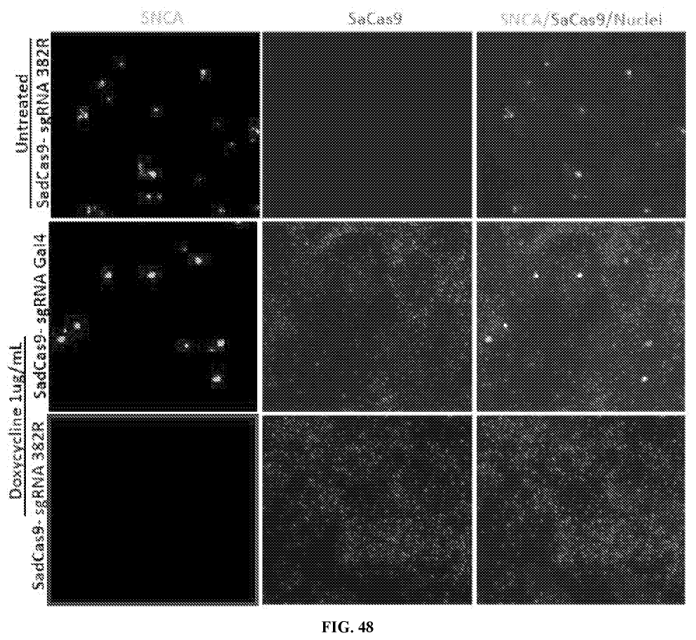

An RNA hybridization technology RNAScope to analyze expression of alpha-synuclein and Cas9 co-expressed in cell culture and brain tissue was optimized to assess downregulation of alpha-synuclein mRNA (FIG. 48). RNAscope® is a novel multiplex nucleic acid in situ hybridization technology. The probe design amplifies target-specific signals but not background noise and can be quantified. In brief, cells are plated in 8-well slide chambers, and treated for 48 h with 1 ug/mL doxycycline to activate sadCas9 expression before fixation (10% NBF) and dehydration with alcohol. SNCA and SadCas9 mRNA are detected by in situ RNA hybridization kit (RNAScope Fluorescent Multiplex Reagent kit, Cat. No. 320850). The SNCA probe targets exon 6, whereas SaCas9 probe targets positions 699-1732 of mRNA sequence. RNAScope treatment consists of a protein-removal step, with sequential incubation of specific target probes (alpha-synuclein/SNCA, Cat.No. 605681 and SaCas9, Cat. No. 501621) for 2 hrs at 40° C., and three subsequent steps of signal amplification and background suppression (15-30 min each, 40° C.), followed by addition of fluorescent-labeled probes (SNCA-green, saCas9-far red). RNA molecules are detected as punctate and are visualized by fluorescence microscopy (Keyence BZ-X700). FIG. 48 represents downregulation of alpha-synuclein by one of the lead sgRNA candidates that show 75% reduction of mRNA transcript by Taqman Q-PCR technology. RNAScope allows us to visualize and confirm human alpha-synuclein downregulation with an independent technology which is only achieved when sadCas9 is co-expressed in the cells after activation with doxycycline. However, when sadCas9 is expressed alone or with an unspecific sgRNA (e.g. Gal4, bacterial target), alpha-synuclein expression is unaffected (FIG. 48).

In Vivo Proof-of-Concept Studies:

All in vivo experiments were conducted under an approved institutional animal welfare protocol (BS-001-2018). 14-17-week-old mice strain PAC-Tg (SNCA A53T); Snca −/− were used. The animals were injected in the tail vain or stereotactically injected in the striatum and substantia nigra. In the experimental groups, there were 6 females and 6 males. For the PBS control, we used 3 females and 3 males.

For stereotactic injections: Stereotactic injections were conducted under 2.5% isofluorane anesthesia. Briefly, mice were positioned in a stereotactic frame (KOPF, David Kopf Instruments—model 900) with a mouse gas anesthesia head holder (KOPF, Model 932-B) and a rodent warmer control box (Stoelting models 53800 and 53800M). After complete sedation mice were placed in an adjustable stage platform (KOPF, model 901) carefully placing the ear bars and securing the head holder for continuous anesthesia. Head fur was carefully shaved and a 1.5 cm incision was made longitudinally with a surgical scalpel. Two holes were drilled using a KOPF drill (KOPF, model 1474) with stereotactic coordinates relative to bregma for striatum (AP +0.5, ML −2.3, DV −3.2) and substantia nigra (AP −3.2, ML −1.4, DV −4.3). One μl virus was then delivered with a Hamilton syringe with a total viral titer of 1.64×109 vg (See Table 6 for groups). The virus was slowly injected for 1 min and then waiting 5 min before taking out the needle to avoid backflow. Control animals were injected with the same volume of phosphate-buffered saline (PBS) as mice in the experimental group in the two sites (striatum and substantia nigra). The wound was closed with 2 stitches of polypropylene 5-0 (Oasis, MedVet international) and a single dose of 0.2 ml of buprenorphine (0.3 mg/ml) was administered I.P. as post-surgical pain relief.

For tail vein injection: 3 male and 2 female mice were anesthetized with 2% isofluorane and held in a tailveiner restrainer (Braintree Scientific, Inc. Cat No. TV-150). The injection was performed with insulin needles (31 gauge) injecting 100 μl of virus with a titer of 2.2×1013 vg/ml (ATCC, Cat. No. 59462-PHPeB).

TABLE 6
Experimental groups for in vivo studies

| Vector | Adminstr. route | # Animals | M:F | Titer | |
|---|---|---|---|---|---|
| pAAV.PHP.eb-U6>sasg382R-hMecp2 | Stereotactic: striatum & SN | 12 | 6:6 | 1.64 × 10⁹ | vg |
| promoter>SadCas9/2xKRAB:SV40 pA | | | | | |
| pAAV.PHP.eb-U6>sasgGal4-hMecp2 | Stereotactic: striatum & SN | 12 | 6:6 | 1.64 × 10⁹ | vg |
| promoter>SadCas9/2xKRAB:SV40 pA | | | | | |
| pAAV.PHP.eb-hMecp2 | Stereotactic: striatum & SN | 12 | 2:2 | 1.64 × 10⁹ | vg |
| promoter>EGFP:SV40 pA | | | | | |
| PBS | Stereotactic: striatum & SN | 6 | 3:3 | | |
| pAAV.PHP.eb-CAG-dTomato | Tail vein injection | 5 | 3:2 | 2.2 × 10¹³ | vg/ml |

Tissue Harvesting

For histology: Mice were intracardially perfused using 80 ml of PBS 0.1M and then 60 ml of 4% buffered PFA using a perfusion needle (BD 0.50 mm× 16 mm syringe) and flow regulator (3.5 ml/min). Brains collected were placed in glass vials with 4% buffered PFA overnight and dehydrated sequentially with 10%, 20% and 30% sucrose solutions overnight at 4° C. After the dehydration cycle, brains were embedded in OCT TissueTek and cut coronally in 40 um slices for further analysis. For brain lysates: Brains was removed from the skull and placed in a mouse brain mold (ASI instruments, Cat. No. RBM-2000C). Once the brain was placed in the mold 3 blades were inserted at the 4th, 6th, and 7th incision (frontal to caudal). After inserting the blades, the brain was removed and placed in wax paper to continue dissecting. The tissue was cut sagittally to have a left and a right hemisphere of all parts of the tissue. In the first section of the brain (anteroposterior (AP) 2 relative to bregma), the olfactory bulb along with a section of the cortex was collected. In the second section from 4-6th incision in the mold, the striatum was collected (anteroposterior 1.8 to −2.2 relative to bregma). The substantia nigra was therefore collected in the 6-7th place (−2.2 to −3.4 anteroposterior relative to bregma). Finally, the cerebellum was separated from the brain stem and collected manually. The rest of the tissue was also collected in a separated tube which include the last part of the mesencephalon, brain stem, retrohippocampal region and hindbrain. All samples were fast frozen in liquid nitrogen and then placed in −80° C. for further analysis.

For tail vain tissue harvesting: Brain was harvested as brain lysates (described above) and complete dissection was performed to obtain tissues of heart, right lung, right kidney, small intestine (jejunum), right lateral lobe of the liver, and testis or uterus and ovaries. All the samples were collected in Eppendorf tubes and fast frozen in liquid nitrogen. They were then placed in -80° C. for further analysis. Left kidney, left lung, left lateral lobe of the liver and part of the jejunum where embedded in OCT Tissue Tek and frozen in dry ice, samples were then stored at -80C for further histological analysis.

Immunohistofluorescence.

For dTomato localization in the coronal sections: Tissue was washed 2 times (5 min each) in PBS and incubated for 6 min in Hoechst 33342 (ThermoFisher, H1399), placed onto Superfrost Plus slides (Thermofisher, 4951PLUS), and mounted with Prolong Diamond Antifade Reagent (Thermofisher, P36970). Microscopy was performed in a KEYENCE BZ-X700 microscope and the images where analyzed with BZ-X700 software. For Immunohistochemistry: Tissue sections were placed in 1 ml 1× immunoretriever (Immuno DNA retriever, BSB 0023) at 65° C. for 35 minutes. After this incubation time, the tissue was placed at room temperature for 15 minutes, washed 3 times with PBT (PBS+0.3% TritonX) and incubated in blocking solution (PBS +0.5% bovine serum albumin +0.5% goat serum). The primary antibody was diluted in blocking solution and incubated overnight at 4° C. After overnight incubation, slices where washed 3 times (10 min each) with PBS and incubated 2 hours with the secondary antibody diluted in PBS. Hoechst 33342 was used as nuclei counterstain and the samples were mounted with Prolong Diamond Antifade reagent. Microscopy was performed in a KEYENCE BZ-X700 microscope and the images where analyzed with BZ-X700 software.

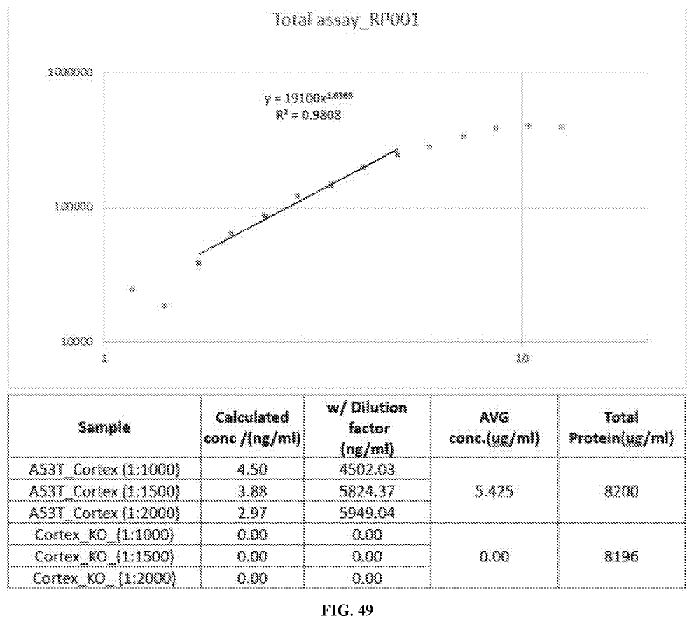

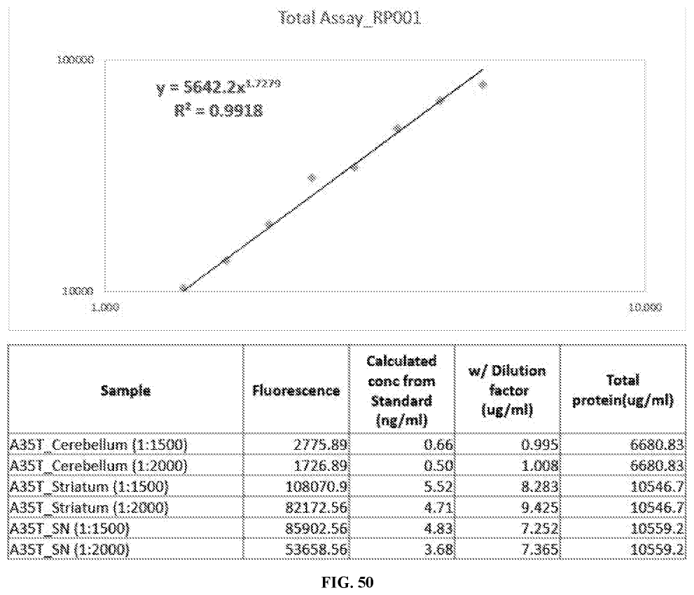

AlphaLisa Protein assay: For the assessment of alpha-synuclein protein levels, a novel assay was established which allows sensitive and specific analysis of total and pS129 human alpha-synuclein species. The assay shows a wide dynamic range permitting a single run even with highly varying human alpha-synuclein levels between samples. With this assay, we can quantitatively measure alpha-synuclein in brain lysates (FIG. 49 and FIG. 50).

Brain samples: After euthanization, different parts of the mouse brain were dissected. Samples were freshly snap-frozen in liquid nitrogen and stored at −80C for at least 24 hours before processing. During lysate preparation, brain samples were lysed with 0.5% NP-40 buffer with protease and phosphatase inhibitor. Lysates were then further manually homogenized with a pestle. After homogenization, ultracentrifugation was used to separate unprocessed tissue from lysate. Bradford protein assay was used to measure total protein content in lysates.

Samples were diluted in 0.01% SDS buffer at 1:15 to 1:1000 depending on cell type and tissue. Lysates from transgenic A53T KO mouse brain were diluted at 1:1000. A standard curve was prepared with monomeric human alpha-synuclein (Proteos, Cat. No. RP001). Both samples and standard were run in triplicates. After dilutions were prepared, 5 ul of samples/standard were added in each well of an optic 384-well plate. Then, 5 ul of anti-alpha-synuclein antibody (Abcam, LB509) conjugated with Europium bead and biotinylated 42/alpha-synuclein antibody (BD Biosciences, Cat. No. 610787) were added to the well and incubated for 90 minutes in the dark. After incubation, 15ul of donor beads were added to each well to initiate energy transfer to detect fluorescence level. Donor beads were incubated for 60 minutes in dark. After the incubation time, fluorescence was measured at 615 nm using Envision multimode plate reader (Perkin Elmer).

All tissues are processed and immunohistofluorescence, RNA Scope, and Taqman expression studies is performed.

### Example 14

### Develop a Target Product Profile

AAV9 vector for the study (Mendell 2017 NEJM, 377;18) is used, but other AAV9 variants can be used with the goal to have a systemic delivery and distribution of the virus in the central and peripheral nervous system.

TABLE 7

| Product Properties | Minimum Acceptable Result | Ideal Result |
|---|---|---|
| Primary Indication | Alpha-synuclein-related Parkinson's disease | Idiopathic Parkinson's disease |
| Patient Population | Patients with parkinsonism and mutations in | Patients with parkinsonism |
| | the alpha-synuclein gene that lead to | |
| | overexpression of alpha-synuclein protein | |
| Treatment Duration | 1-time stereotactic surgery | 1-time intravenous delivery |
| Delivery Mode | Surgical delivery into substantia nigra of CNS | Intravenous delivery |
| | or intrathecally | |
| Dosage Form | Viral vector AAV9 or AAV- | Viral vector AAV9 or AAV- |
| | PHP-.eB::SadCas9-KRAB- | PHP.eB::SadCas9-KRAB- |
| | sgRNA_SNCA1 at 2 × 10\hat{ }14 | sgRNA_SNCA1 at2 × 10\hat{ }13 |
| Regimen | N/A | N/A |
| Efficacy | Slowdown of disease progression of UPDRS | Stopping disease progression of |
| | motor score | motor symptoms of parkinsonism |
| Risks/Side Effect | Side effects related to surgery, such as | Side effects related to surgery, such |
| | headaches, procedural pain, intracranial | as headaches, procedural pain, |
| | hemorrhage, stroke, memory problems | intracranial hemorrhage, stroke, |
| | | memory problems |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of modifying expression of alpha-synuclein (SNCA) gene in an individual in need thereof, the method comprising: administering to the individual a composition comprising (i) at least one synthetic polynucleotide that targets a target sequence in one or more of the SNCA genes, and (ii) a nucleic acid-guided nuclease, wherein targeting the target sequence represses the transcription of one or more SNCA genes, thereby modifying expression of the SNCA gene in the individual.

2. The method of claim 1, wherein the individual has a neurodegenerative disease.

3. The method of claim 1, wherein the individual has Parkinson's disease, Parkinson's-related disease, or synucleinopathy.

4. The method of claim 1, wherein the individual overexpresses the SNCA gene.

5. The method of claim 1, wherein the individual has more than two copies of a functional SNCA gene.

6. The method of claim 1, wherein the individual has three copies of a functional SNCA gene.

7. The method of claim 1, wherein the individual has four copies of a functional SNCA gene.

8. The method of claim 1, wherein the synthetic polynucleotide is a guide nucleic acid.

9. The method of claim 8, wherein the guide nucleic acid is a guide RNA (gRNA).

10. The method of claim 1, wherein the synthetic polynucleotide comprises a transcriptional start site of one or more SNCA genes.

11. The method of claim 1, wherein the target sequence is in the promoter region of one or more SNCA genes.

12. The method of claim 1, wherein the target sequence is proximal to a transcriptional start site of one or more SNCA genes.

13. The method of claim 1, wherein the nucleic acid-guided nuclease is a CR1SPR nuclease.

14. The method of claim 13, wherein the CR1SPR nuclease is Cas9.

15. The method of claim 13, wherein the CR1SPR nuclease is bacterial Cas9.

16. The method of claim 15, wherein the bacterial Cas9 is from Staphylococcus aureus.

17. The method of claim 1, wherein the nucleic acid-guided nuclease is catalytically inactive.

18. The method of claim 1, wherein the transcription is repressed by interfering with transcription initiation, transcription elongation, RNA polymerase binding, transcription factor binding, or any combination thereof.

19. The method of claim 1, wherein repressing the transcription of one or more SNCA genes is reversible.

20. The method of claim 1, wherein repressing the transcription of one or more SNCA genes decreases the expression of the SNCA gene in the individual.

21. The method of claim 20, wherein the decreased expression of the SNCA gene is comparable to the expression of SNCA gene in a control cell.

22. The method of claim 1, wherein the modified expression of SNCA gene is comparable to the expression of SNCA gene in a control cell.

23. The method of any one of claim 21 or 22, wherein the control cell comprises two copies of functional SNCA gene.

24. The method of claim 1, wherein the transcription of SNCA gene is repressed by at least 50% compared to transcription of SNCA gene before administration of the composition.

25. A method of treating a neurodegenerative disease in an individual in need thereof, the method comprising: administering to the individual a composition comprising (i) at least one synthetic polynucleotide that targets a target sequence in one or more of the SNCA genes, and (ii) a nucleic acid-guided nuclease,
wherein the individual overexpresses SNCA gene, and wherein targeting the target sequence represses the transcription of one or more SNCA genes, thereby treating the individual.

26. The method of claim 25, wherein the neurodegenerative disease is Parkinson's disease, Parkinson's-related disease, or synucleinopathy.

27. The method of claim 25, wherein the individual has more than two copies of a functional SNCA gene.

28. The method of claim 25, wherein the individual has three copies of a functional SNCA gene.

29. The method of claim 25, wherein the individual has four copies of a functional SNCA gene.

30. The method of claim 25, wherein the synthetic polynucleotide is a guide nucleic acid.

31. The method of claim 30, wherein the guide nucleic acid is a guide RNA (gRNA).

32. The method of claim 25, wherein the synthetic polynucleotide comprises a transcriptional start site of one or more SNCA genes.

33. The method of claim 25, wherein the target sequence is in the promoter region of one or more SNCA genes.

34. The method of claim 25, the target sequence is proximal to a transcriptional start site of one or more SNCA genes.

35. The method of claim 25, wherein the nucleic acid-guided nuclease is a CRISPR nuclease.

36. The method of claim 35, wherein the CRISPR nuclease is Cas9.

37. The method of claim 35, the CRISPR nuclease is bacterial Cas9.

38. The method of claim 37, wherein the bacterial Cas9 is from Staphylococcus aureus.

39. The method of claim 25, wherein the nucleic acid-guided nuclease is catalytically inactive.

40. The method of claim 25, wherein the transcription is repressed by interfering with transcription initiation, transcription elongation, RNA polymerase binding, transcription factor binding, or any combination thereof.

41. The method of claim 25, wherein repressing the transcription of one or more SNCA genes is reversible.

42. The method of claim 25, wherein repressing the transcription of one or more SNCA genes decreases the expression of the SNCA gene in the individual.

43. The method of claim 42, wherein the decreased expression of the SNCA gene is comparable to the expression of SNCA gene in a control cell.

44. The method of claim 43, wherein the control cell comprises two copies of functional SNCA gene.

45. A method of measuring efficacy of a treatment for neurodegenerative disease in an individual overexpressing SNCA gene, the method comprising:
(a) determining the copy number of SNCA gene in the individual;
(b) contacting an isogenic induced pluripotent cell comprising a copy number of SNCA gene the same as the individual with a composition comprising (i) at least one synthetic polynucleotide that targets a target sequence in one or more SNCA genes, and (ii) a nucleic acid-guided nuclease;
(c) detecting the response in the cell; and
(d) comparing said response to control cells.

46. The method of claim 45, further comprising (e) adjusting the treatment to get a response comparable to the control cells.

47. The method of claim 45, further comprising (f) administering the composition with efficacy for treatment of the neurodegenerative to the individual.

48. The method of claim 45, wherein the neurodegenerative disease is Parkinson's disease, Parkinson's-related disease, or synucleinopathy.

49. The method of claim 45, wherein the individual has more than two copies of a functional SNCA gene.

50. The method of claim 45, wherein the individual has three copies of a functional SNCA gene.

51. The method of claim 45, wherein the individual has four copies of a functional SNCA gene.

52. The method of claim 45, wherein the synthetic polynucleotide is a guide nucleic acid.

53. The method of claim 52, wherein the guide nucleic acid is a guide RNA (gRNA).

54. The method of claim 45, wherein the synthetic polynucleotide comprises a transcriptional start site of one or more SNCA genes.

55. The method of claim 45, wherein the target sequence is in the promoter region of one or more SNCA genes.

56. The method of claim 45, wherein the target sequence is proximal to a transcriptional start site of one or more SNCA genes.

57. The method of claim 45, wherein the nucleic acid-guided nuclease is a CRISPR nuclease.

58. The method of claim 57, wherein the CRISPR nuclease is Cas9.

59. The method of claim 57, wherein the CRISPR nuclease is bacterial Cas9.

60. The method of claim 59, wherein the bacterial Cas9 is from Staphylococcus aureus.

61. The method of claim 45, wherein the nucleic acid-guided nuclease is catalytically inactive

62. The method of claim 45, wherein targeting the target sequence represses the transcription of one or more SNCA genes.

63. The method of claim 62, wherein the transcription is repressed by interfering with transcription initiation, transcription elongation, RNA polymerase binding, transcription factor binding, or any combination thereof.

64. The method of claim 62, wherein repressing the transcription of one or more SNCA genes is reversible.

65. The method of claim 45, wherein the response is change in cell viability, cellular chemistry, cellular function, mitochondrial function, cell aggregation, cell morphology, cellular protein aggregation, gene expression, cellular secretion, cellular uptake, or combinations thereof.

66. The method of claim 45, wherein the response is detecting expression of one or more SNCA genes.

67. The method of claim 45, wherein the control cell is an isogenic induced pluripotent cell comprising a copy number of SNCA gene the same as the individual without contact with the composition, or wherein the control cell is an isogenic induced pluripotent cell comprising two functional copies of SNCA gene without contact with the composition, or both.

68. A pharmaceutical composition for treatment of a neurodegenerative disease in an individual in need thereof, comprising (i) at least one synthetic polynucleotide that targets a target sequence in one or more of SNCA genes, and (ii) a nucleic acid-guided nuclease; and a pharmaceutically-acceptable excipient,
wherein the composition has efficacy in the treatment of the neurodegenerative disease, wherein said efficacy is measured according to the method of claim 45.

69. The pharmaceutical composition of claim 68, wherein the neurodegenerative disease is Parkinson's disease, Parkinson's-related disease, or synucleinopathy.

70. The pharmaceutical composition of claim 68, wherein the individual overexpresses the SNCA gene.

71. The pharmaceutical composition of claim 68, wherein the individual has more than two copies of a functional SNCA gene.

72. The pharmaceutical composition of claim 68, wherein the individual has three copies of a functional SNCA gene.

73. The pharmaceutical composition of claim 68, wherein the individual has four copies of a functional SNCA gene.

74. The pharmaceutical composition of claim 68, wherein the synthetic polynucleotide is a guide nucleic acid.

75. The pharmaceutical composition of claim 74, wherein the guide nucleic acid is a guide RNA (gRNA).

76. The pharmaceutical composition of claim 68, wherein the synthetic polynucleotide comprises a transcriptional start site of one or more SNCA genes.

77. The pharmaceutical composition of claim 68, wherein the target sequence is in the promoter region of one or more SNCA genes.

78. The pharmaceutical composition of claim 68, wherein the target sequence is proximal to a transcriptional start site of one or more SNCA genes.

79. The pharmaceutical composition of claim 68, wherein the nucleic acid-guided nuclease is a CRISPR nuclease.

80. The pharmaceutical composition of claim 79, wherein the CRISPR nuclease is Cas9.

81. The pharmaceutical composition of claim 79, wherein the CRISPR nuclease is bacterial Cas9.

82. The pharmaceutical composition of claim 81, wherein the bacterial Cas9 is from Staphylococcus aureus.

83. The pharmaceutical composition of claim 68, wherein the nucleic acid-guided nuclease is catalytically inactive.

84. A method of modifying expression of alpha-synuclein (SNCA) gene in an induced pluripotent stem cell, the method comprising:
(a) providing induced pluripotent stem cell that overexpresses SNCA gene; and
(b) contacting the stem cell with (i) at least one synthetic polynucleotide that targets a target sequence in one or more of the SNCA genes, and (ii) a nucleic acid-guided nuclease, wherein targeting the target sequence represses the transcription of one or more SNCA genes, thereby modifying expression of the SNCA gene.

85. The method of claim 84, wherein the cell has more than two copies of a functional SNCA gene.

86. The method of claim 84, wherein the cell has three copies of a functional SNCA gene.

87. The method of claim 84, wherein the cell has four copies of a functional SNCA gene.

88. The method of claim 84, wherein the synthetic polynucleotide is a guide nucleic acid.

89. The method of claim 88, wherein the guide nucleic acid is a guide RNA (gRNA).

90. The method of claim 84, wherein the synthetic polynucleotide comprises a transcriptional start site of one or more SNCA genes.

91. The method of claim 84, wherein the target sequence is in the promoter region of one or more SNCA genes.

92. The method of claim 84, wherein the target sequence is proximal to a transcriptional start site of one or more SNCA genes.

93. The method of claim 84, wherein the nucleic acid-guided nuclease is a CRISPR nuclease.

94. The method of claim 93, wherein the CRISPR nuclease is Cas9.

95. The method of claim 93, wherein the CRISPR nuclease is bacterial Cas9.

96. The method of claim 95, wherein the bacterial Cas9 is from Staphylococcus aureus.

97. The method of claim 84, wherein the nucleic acid-guided nuclease is catalytically inactive.

98. The method of claim 84, wherein the transcription is repressed by interfering with transcription initiation, transcription elongation, RNA polymerase binding, transcription factor binding, or any combination thereof.

99. The method of claim 84, wherein repressing the transcription of one or more SNCA genes is reversible.

100. The method of claim 84, wherein repressing the transcription of one or more SNCA genes decreases the expression of the SNCA gene in the cell.

101. The method of claim 100, wherein the decreased expression of the SNCA gene is comparable to the expression of SNCA gene in a control cell.

102. The method of claim 84, wherein the modified expression of SNCA gene is comparable to the expression of SNCA gene in a control cell.

103. The method of any one of claim 101 or 102, wherein the control cell comprises two copies of functional SNCA gene.

104. The method of claim 84, wherein the cell is present in a cell culture.

105. The method of claim 89, wherein the gRNA modifies the expression of the SNCA gene by suppressing the expression of the SNCA gene by 75%.

106. The method of claim 89, wherein the gRNA modifies the expression of the SNCA gene by suppressing the expression of the SNCA gene by 50%.

107. The method of claim 89, wherein the gRNA is a gRNA according to the sequence CTCCTCTGGGGACAGTCCCCC (382R).

108. The method of claim 89, wherein the gRNA is a gRNA according to the sequence AAGAGAGAGGCGGGGAGGAGT (267R).

109. The method of claim 89, wherein the gRNA is a gRNA according to the sequence GAATGGTCGTGGGCACCGGGA (155R).
